(19) United States
(12) Patent Application Publication
(10) Pub. No.: US 2024/0150444 A1
(43) Pub. Date: May 9, 2024

(54) PROTECTIVE ANTIBODIES AGAINST RESPIRATORY VIRAL INFECTIONS

(71) Applicant: Fred Hutchinson Cancer Center, Seattle, WA (US)

(72) Inventors: Justin J. Taylor, Seattle, WA (US); Jim Boonyaratanakornkit, Seattle, WA (US)

(73) Assignee: Fred Hutchinson Cancer Center, Seattle, WA (US)

(21) Appl. No.: 18/548,017

(22) PCT Filed: Feb. 25, 2022
(86) PCT No.: PCT/US2022/017945

§ 371 Date: Aug. 25, 2023

Related U.S. Application Data
(60) Provisional application No. 63/154,166, filed on Feb. 26, 2021.

(51) Int. Cl. C07K 16/10; A61P 31/12

(52) U.S. Cl. CPC C07K 16/1027; A61P 31/12; A61K 38/00; C07K 2317/33; C07K 2317/55; C07K 2317/565; C07K 2317/622; C07K 2317/76

(57) ABSTRACT

Antibodies that protect against respiratory viral infections including human parainfluenza viruses (HPIV), respiratory syncytial virus (RSV), and human metapneumovirus (HMPV) are described. Certain antibodies disclosed herein neutralize more than one virus (e.g., RSV and HMPV). The binding fragments of the antibodies can be engineered into numerous formats including those that provide simultaneous protection against multiple viruses.

### CROSS-REFERENCE TO RELATED APPLICATION

This application is a U.S. National Phase Patent Application based on International Patent Application No. PCT/US2022/017945, filed Feb. 25, 2022, which claims priority to U.S. Provisional Patent Application No. 63/154,166 filed Feb. 26, 2021, both of which are incorporated herein by reference in their entirety as if fully set forth herein.

### REFERENCE TO SEQUENCE LISTING

The Sequence Listing associated with this application is provided in text format in lieu of a paper copy and is hereby incorporated by reference into the specification. The name of the text file containing the Sequence Listing is 2YB9998_ST25.txt. The text file is 70,131 bytes, was created on Aug. 25, 2023, and is being submitted electronically via Patent Center.

### FIELD OF THE DISCLOSURE

The current disclosure provides antibodies that protect against respiratory viral infections including human parainfluenza viruses (HPIV), respiratory syncytial virus (RSV), and human metapneumovirus (HMPV). Certain antibodies disclosed herein neutralize more than one virus (e.g., RSV and HMPV). The binding fragments of the antibodies can be engineered into numerous formats including those that provide simultaneous protection against multiple viruses.

### BACKGROUND OF THE DISCLOSURE

Respiratory paramyxo/pneumo-viral infections result in the deaths of tens of thousands of people in the US each year. Patients receiving hematopoietic stem cell transplants (HCT) are especially vulnerable during the period it takes for their immune system to repopulate. The human parainfluenza viruses (HPIV), respiratory syncytial virus (RSV), and human metapneumovirus (HMPV) can be the deadliest following HCT. Unfortunately, there are no preventative or treatment options for RSV, HMPV, and HPIV infections in the elderly or HCT recipients. The antibody palivizumab is given prophylactically to premature infants to protect against RSV, but nothing is available for HMPV and the HPIVs.

### SUMMARY OF THE DISCLOSURE

The current disclosure provides new antibodies that are protective against one or more of human parainfluenza viruses (HPIV1 and/or HPIV3), respiratory syncytial virus (RSV), and human metapneumovirus (HMPV). Particular antibodies disclosed herein are protective against HPIV3. Other antibodies disclosed herein are protective against HPIV3 and HPIV1. Other antibodies disclosed herein are protective against RSV and HMPV. The newly disclosed antibodies can be used to protect against these various forms of respiratory viruses.

In particular embodiments, binding fragments from these newly-disclosed antibodies and others can be engineered into multi-specific formats that offer protection from multiple viruses at a time including multiple additional respiratory viruses. In particular embodiments, these antibodies and engineered formats thereof provide simultaneous protection against multiple medically important respiratory viruses that afflict children, the elderly, and the immunocompromised.

PI3 antibodies disclosed herein bind and neutralize HPIV3. The “3×1” antibody disclosed herein binds and neutralizes HPIV3 and HPIV1. The MxR antibodies disclosed herein bind and neutralize RSV and HMPV.

### BRIEF DESCRIPTION OF THE SEVERAL VIEWS OF THE DRAWINGS

Many of the drawings submitted herein may be better understood in color. Applicant considers the color versions of the drawings as part of the original submission and reserves the right to present color images of the drawings in later proceedings.

FIG. 1. Exemplary use of antibodies disclosed herein to bridge windows of vulnerability in hematopoietic stem cell transplant recipients.

FIGS. 2A-21. Screening human PBMCs, tonsils, and spleen for HPIV3-specific B cells. 2A) HPIV3-specific B cells were labeled with APC-conjugated streptavidin tetramers of biotinylated HPIV3 fusion (F) protein followed by magnetic enrichment using microbeads against APC. Representative flow cytometry plot of enriched HPIV3-specific B cells after gating on live (fixable viability dye negative), CD3/14/16⁻, and CD19⁺ B cells. Cells in the grey box of the enriched fraction are B cells that bind the prefusion (preF) but not postfusion (postF) conformation of the HPIV3 F protein. The percentage indicates the percentage of total cells shown in the flow plot. 2B) The frequency HPIV3 preF-specific B cells in human PBMCs (N=2 donors), tonsils (N=2 donors), and spleen (N=2 donors). 2C, 2D) Representative flow cytometry plots of switched (IgM⁻/IgD⁻) B cells as a percentage of total B cells in PBMCs (N=2 donors), tonsils (N=2 donors) and spleen (N=2 donors). 2E, 2F) The frequency of switched (IgM⁻/IgD⁻) HPIV3 preF-specific B cells as a percentage of total B cells in PBMCs (N=2 donors), tonsils (N=2 donors), and spleen (N=2 donors). 2G) Biolayer interferometry measurements of association and dissociation between HPIV3 preF and 25 monoclonal antibodies (mAbs) cloned directly from individually sorted HPIV3 preF-specific B cells. 2H) Binding kinetics of the PI3-E12 and PI3-C9 mAbs with HPIV3 preF measured by biolayer interferometry. I) Plaque reduction neutralization test of the PI3-E12 mAb. Asterisks Indicate P<0.05 by t-Test Compared to PBMCs.

FIGS. 3A-3F. Higher throughput screening of human PBMCs, tonsils, and spleen for B cells capable of producing neutralizing antibodies against HPIV3. (3A) Detection of IgG by ELISA in supernatant from random B cells (N=2 independent experiments) individually sorted and subsequently expanded on feeder cells. (3B) Plaque reduction neutralization test of supernatant from HPIV3 preF-specific B cells individually sorted and subsequently expanded on feeder cells (N=120 cells for PBMCs, N=120 cells for tonsils, N=1,235 cells for spleen). (3C) Neutralizing titers of HPIV3-specific monoclonal antibodies were determined by 60% plaque reduction neutralization tests on Vero cells using GFP-labeled HPIV3. Penta-HIS probes were loaded with either the preF (3D) or postF (3E) conformation of HPIV3 F. Association with each mAb was then measured by biolayer interferometry. All measurements are normalized against a negative control antibody. The positive control antibody is a human mAb known to bind HPIV3 postF. (3F) Anti-nuclear antibody assay in HEp-2 cells using mAbs targeting HPIV3 preF. Binding was detected using a secondary Alexa Fluor 647 (AF647)-conjugated goat anti-human antibody. The mAbs palivizumab was used as a negative control and 2F5 as a positive control for auto-reactivity. The average fluorescence intensity was calculated from two independent experiments.

FIGS. 4A-4D. Structural analysis of monoclonal antibodies against HPIV3 preF. (4A) Negative stain electron microscopy (EM) 2D classifications of HPIV3 preF in complex with PI3-E12 F_{ab}. 4B) Negative stain EM 3D reconstruction. Coordinates of HPIV3 preF trimer (labeled, PDB ID 6MJZ), trimeric domain GCN4 (labeled, PDB ID 4DME), and crystal structure of PI3-E12 F_{ab} (labeled, this paper) were fitted in the 3D map. 4C) Structure of PI3-E12 F_{ab} with CDRs labeled. Left, cartoon representation. Right, surface representation. 4E) Structural alignment with PIA174 (indicated with stars). Dots in CDR sequences represent gaps based on IMGT unique numbering.

FIGS. 5A-5E. Efficacy of prophylactic and therapeutic administration of a neutralizing HPIV3 mAb in vivo. (5A) Schematic of the prophylaxis experiments performed in cotton rats (N=4 animals per group). (5B) Lung histopathology at day four post-infection in cotton rats. The arrow indicates an area of peri-bronchiolitis. Peribronchiolitis was scored as percent severity. (5C) Viral titers by plaque assay in nasal and lung homogenates at day four post-infection. (5D) Schematic of the therapeutic experiments performed in immunocompromised cotton rats (N=5 animals per group). (5E) Viral titers by plaque assay in nasal (top) and lung (bottom) homogenates at day four post-infection. Asterisks indicate P<0.05 by t-test compared to the PBS control.

FIGS. 6A, 6B. Dual binding and neutralization by the MxR-class mAbs. (6A) Detection of binding between the MxR-01 mAb with RSV preF (top) and HMPV preF (bottom) by biolayer interferometry. The MxR-01 antibody is also referred to as MxR-B11. (6B) Neutralizing titers of MxR-01 and palivizumab for RSV and HMPV were determined by a plaque reduction neutralization assay. MxR-01 potently prevented infection by HMPV and RSV in vitro with low neutralization titers 0.07 μg/mL. In this assay MxR-01 outperformed RSV neutralization by palivizumab by almost 10-fold.

FIG. 7. Depictions of engineered antibody formats and representative uses.

FIG. 8. Design of quad-protective antibodies by engineering into a single monoclonal antibody the antigen binding sites of MxR-01 and 3×1 using alternative bispecific formats. The depicted configurations are generated using (top) the knobs-into-holes approach, which promotes heterodimerization to produce an Ab connected by the fragment constant (Fc) region but containing one antigen-binding fragment (Fab) from MxR-01 and another from 3×1; and (bottom) an IgG-scFv fusion in which the scFv of one antibody is linked to the constant region of the other antibody.

FIGS. 9A-9C. Isolation of HMPV/RSV (MxR) and HPIV3/HPIV1 (3×1) cross-neutralizing mAbs. (9A) RSV- and HMPV-binding B cells from human blood were labeled with APC-conjugated streptavidin tetramers of biotinylated RSV prefusion protein (preF) and PE-conjugated streptavidin tetramers of biotinylated HMPV preF. Flow cytometry plot of RSV and HMPV preF-binding B cells after gating for live, CD3⁻CD14⁻CD16⁻ (to exclude non-B cells), CD19⁺CD20⁺ (B cells), IgD⁻/IgM⁻ (isotype-switched), and APC/Dylight755-HMPV postF-PE/DL650-RSV postF⁻ (decoy to exclude cells binding to RSV/HMPV postF, APC, PE, or streptavidin). The grey box indicates the B cell from which MxR was derived. (9B) HPIV3-binding B cells from human spleen were labeled with APC-conjugated streptavidin tetramers of biotinylated HPIV3 preF. Flow cytometry plot of HPIV3-binding B cells after gating for live, CD3⁻CD14⁻CD16⁻, CD19³⁰ CD20⁺, and IgD⁻/IgM⁻. Cells in the black box of the bound fraction are B cells that bind HPIV3 preF but not postF. The grey box indicates the B cell from which 3×1 was derived. (9A, 9B) The bound fraction represents magnetically enriched cells using microbeads against allophycocyanin (APC) and phycoerythrin (PE). Numbers in gates are percentages of total cells in the flow plot. (9C) Schematic of the “bait-and-switch” approach using a single antigen to identify cross-neutralizing B cells. Two lead candidates MxR which neutralizes RSV and HMPV and 3×1 which neutralizes HPIV3 and HPIV1 were identified using this approach.

FIGS. 10A-10C. B cell phenotype and potency of MxR and 3×1 cross-neutralizing mAbs. (10A) Source, phenotype, and allele usage of the B cells that produced MxR and 3×1. (10B) Vero cells were infected with RSV in the presence of serial dilutions of palivizumab (Pali.) and MxR. The upper dotted line indicates the PRNT₆₀. (10C) PRNT₆₀ of palivizumab against RSV; MxR against RSV-A, RSV-B, and HMPV; and 3×1 against HPIV1 and HPIV3. Data points are from three independent experiments with each experiment consisting of two technical replicates. The asterisk indicates a P value <0.01 compared to palivizumab using an unpaired two-tailed t-test with Welch's correction. MxR and 3×1 have in vitro neutralizing potencies that exceed palivizumab against their respective viruses.

FIGS. 11A-11C. Epitope binning and structural analysis. (11A) Penta-HIS probes were coated with His-tagged HPIV3 preF. The mAb listed on the left side of the chart was loaded onto the coated probe followed by the mAb listed on the top. The legend describes the indications for the level of competition between antibodies, expressed as the percent drop in maximum signal of the top mAb in the presence of the left mAb, compared to the maximum signal of the top mAb alone. Site 0 is located at the apex of HPIV3 preF³⁶. Site X is a newly discovered antigenic site. CryoEM structures of (11B) 3×1 in complex with HPIV3 preF at 3.7 Å resolution and of (11C) MxR in complex with RSV preF at 2.3 Å resolution. Top views are on the left. Side views are on the right. 3×1 binds to a novel antigenic site on HPIV3 preF. MxR binds at the equator of RSV preF.

FIG. 12. In vitro resistance analysis. HPIV3 or RSV was inoculated on Vero cells in the presence of escalating concentrations of 3×1, MxR, or palivizumab (Pali.). For each passage, supernatant was collected five days post-infection from wells under the greatest antibody selection with detectable viral replication, as measured by CPE, and inoculated onto fresh Vero cells in the presence of escalating concentrations of mAb. MxR and HPIV3 may have a higher barrier of resistance compared to palivizumab.

FIG. 13. Biolayer interferometry measurement of binding kinetics between MxR and RSV preF. BLI assays were performed on the Octet.Red instrument (ForteBio) at room temperature with shaking at 500 rpm. Penta-His capture sensors (ForteBio, cat #18-5120) were loaded in kinetics buffer containing 1 μM His-tagged F for 300 s. After loading, the baseline signal was recorded for 60 s in kinetics buffer. The sensors were then immersed in kinetics buffer containing purified antibody for a 300 s association step followed by immersion in kinetics buffer for an additional 1200 s dissociation phase. Curve fitting was performed using a 1:1 binding model and ForteBio data analysis software. MxR binds to the RSV preF protein with exceptionally high affinity, beyond the limit of measurement.

FIG. 14. Biolayer interferometry measurement of binding kinetics between MxR and HMPV preF. BLI assays were performed on the Octet.Red instrument (ForteBio) at room temperature with shaking at 500 rpm. Penta-His capture sensors (ForteBio, cat #18-5120) were loaded in kinetics buffer containing 1 μM His-tagged F for 300 s. After loading, the baseline signal was recorded for 60 s in kinetics buffer. The sensors were then immersed in kinetics buffer containing purified antibody for a 300 s association step followed by immersion in kinetics buffer for an additional 600 s dissociation phase. Curve fitting was performed using a 1:1 binding model and ForteBio data analysis software. MxR binds to the HMPV preF protein with nanomolar affinity.

FIG. 15. Biolayer interferometry measurement of binding kinetics between 3×1 and HPIV3 preF. BLI assays were performed on the Octet.Red instrument (ForteBio) at room temperature with shaking at 500 rpm. Penta-His capture sensors (ForteBio, cat #18-5120) were loaded in kinetics buffer containing 1 μM His-tagged F for 300 s. After loading, the baseline signal was recorded for 60 s in kinetics buffer. The sensors were then immersed in kinetics buffer containing purified antibody for a 300 s association step followed by immersion in kinetics buffer for an additional 1200 s dissociation phase. Curve fitting was performed using a 1:1 binding model and ForteBio data analysis software. 3×1 binds to the HPIV3 preF protein with exceptionally high affinity, beyond the limit of measurement.

FIG. 16. Suppression of RSV replication in the lungs and nasal turbinates of hamsters with prophylactic administration of MxR. Hamster challenge experiments were performed in groups of N=4-5. Hamsters were infected intranasally with 100 μL of 10⁵ pfu virus. MAb (5 mg/kg) or PBS control was administered intramuscularly 2 days prior to infection. Nasal turbinates and lungs were removed for viral titration by plaque assay at day five post-infection. Lung and nose homogenates were clarified by centrifugation. Confluent Vero cell monolayers were inoculated in duplicate with diluted homogenates in 24-well plates. After incubating for 1 hour at 37° C., wells were overlaid with methylcellulose. After 4 days, plaques were counted to determine titers as pfu per gram of tissue. Prophylactic administration of MxR suppresses RSV replication in the nose and lungs of hamsters.

FIG. 17. Suppression of HPIV3 replication in the lungs and nasal turbinates of hamsters with prophylactic administration of 3×1. Hamster challenge experiments were performed in groups of N=4-5. Hamsters were infected intranasally with 100 μL of 10⁵ pfu virus. Mab (5 mg/kg) or PBS control was administered intramuscularly 2 days prior to infection. Nasal turbinates and lungs were removed for viral titration by plaque assay at day five post-infection. Lung and nose homogenates were clarified by centrifugation. Confluent Vero cell monolayers were inoculated in duplicate with diluted homogenates in 24-well plates. After incubating for 1 hour at 37° C., wells were overlaid with methylcellulose. After 4 days, plaques were counted to determine titers as pfu per gram of tissue. Prophylactic administration of 3×1 suppresses HPIV3 replication in the lungs of hamsters.

FIG. 18. Suppression of RSV and HPIV3 co-infection in the lungs and nasal turbinates of hamsters with prophylactic administration of MxR and 3×1 (each at 5 mg/kg). Hamster challenge experiments were performed in groups of N=4-5. Hamsters were infected intranasally with 100 μL containing 10⁵ pfu of each virus. MAb or PBS control was administered intramuscularly 2 days prior to infection. Nasal turbinates and lungs were removed for viral titration by plaque assay at day five post-infection. Lung and nose homogenates were clarified by centrifugation. Confluent Vero cell monolayers were inoculated in duplicate with diluted homogenates in 24-well plates. After incubating for 1 hour at 37° C., wells were overlaid with methylcellulose. After 4 days, plaques were counted to determine titers as pfu per gram of tissue. Prophylactic administration of a cocktail of MxR and 3×1 suppresses the replication of RSV and HPIV3 in a hamster co-infection model.

FIGS. 19A-19E. (19A) Schematic of suppression of RSV and HPIV3 replication in the lungs and nasal turbinates of co-infected hamsters with prophylactic administration of MxR and 3×1 (each at 5 mg/kg). Hamster challenge experiments were performed in groups of N=6-10. Hamsters were infected intranasally with 100 μL of 10⁵ pfu of each virus. MAb or PBS control was administered intramuscularly 2 days prior to infection. Nasal turbinates and lungs were removed for viral quantitation by real-time PCR at day five post-infection. Lung and nose homogenates were clarified by centrifugation. Viral RNA was extracted from sample homogenate using the QIAamp vRNA Mini Kit. Custom reverse transcription primers for RSV (TCCAGCAAATACACCATCCAAC (SEQ ID NO: 187)) and HPIV3 (CTAGAAGGTCAAGAAAAGGGAACTC (SEQ ID NO: 188)) were used for reverse transcription. Custom virus-specific Taqman probes were generated for real-time PCR. 20× Gene Expression Assays for RSV (forward primer TGACTCTCCTGATTGTGGGATGATA (SEQ ID NO: 189), reverse primer CGGCTGTAAGACCAGATCTGT (SEQ ID NO: 190), and reporter CCCCTGCTGCTAATTT (SEQ ID NO: 191)) and HPIV3 (forward primer, CGGTGACACAGTGGATCAGATT (SEQ ID NO: 192), reverse primer TGTTTCAACCATAAGAGTTACCAAGCT (SEQ ID NO: 193), and reporter ACCGCATGATTGACCC (SEQ ID NO: 194)) were used with Taqman Universal mastermix II with UNG. Samples were run on the QuantStudio 7 Flex Real-Time PCR System. Quantitation was based on generating a standard curve with vRNA extracted from viral stocks of RSV and HPIV3 with known titers. Cycle thresholds were interpolated and used to calculate viral titers in pfu/g. Quantification of virus for PBS or Mab cocktail are shown for (19B) RSV Lungs, (19C) RSV Nasal Turbinates, (19D) HPIV3 Lungs, and (19E) HPIV3 Nasal Turbinates. Asterisks indicate P<0.01 by a Mann-Whitney test.

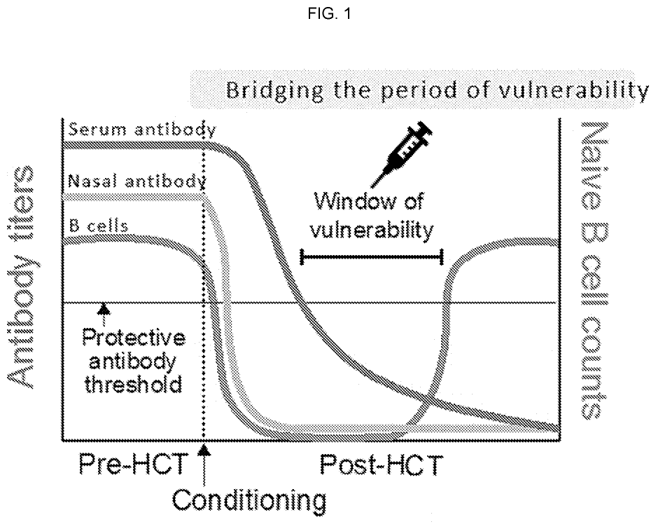

### DETAILED DESCRIPTION

Respiratory paramyxo/pneumo-viral infections result in the deaths of tens of thousands of people in the US and hundreds of thousands of people in the world each year. In addition to contributing to significant health care costs, these infections are particularly morbid and deadly in the immunocompromised as well as high-risk adults and infants.

HCT recipients: Patients receiving hematopoietic stem cell transplant (HCT) are especially vulnerable during the 6-month period it takes for their immune system to repopulate. Over 50,000 HCTs are performed worldwide, with 20,000 occurring in the US. Up to a third of HCT recipients acquire a respiratory viral infection within six months of transplant. In up to a third of those patients, the virus progresses from the upper to the lower respiratory tract. Once the virus gains a foothold in the lower tract, little can be done for most viruses beyond supportive care. As a result, up to 40% of patients with lower tract disease die within three months. Collectively, RSV, HMPV, and the HPIVs account for half of the serious respiratory viral infections after HCT. Of patients who survive the acute infection, over 25% develop fixed air flow obstruction, a debilitating condition of accelerated lung function loss associated with increased mortality.

Lung transplant recipients: Lung transplant recipients represent another immunocompromised population at particularly high risk for morbidity and death. Worldwide, over 3,000 lung transplants are performed annually with 2,000 occurring in the US. Up to 25% of lung transplant recipients become infected by RSV, HMPV, or HPIV, with most infections occurring after the first year of transplant when patients resume community activities. Infection by these viruses can lead to bacterial superinfection, tissue rejection, and chronic allograft dysfunction.

Adults: Individuals over 65 and those with homelessness or chronic lung diseases are at risk for developing severe lung disease. Almost 50 million people in the US are over 65, making up 15% of the population. 550,000 people in the US are homeless and an estimated 24 million adults are living with chronic obstructive pulmonary disease (COPD) in the US.

Infants: Prematurity is a well-known risk factor for developing severe lung disease after infection by RSV. 11% of live births in the US occur at less than 37 weeks' gestation. Infection in the first years are later associated with long-term pulmonary consequences and diminished lung function. RSV, followed by HMPV and then by the HPIVs, account for the vast majority of hospitalizations for viral lower respiratory tract infection.

There are currently no clinically available preventative or treatment options for RSV, HMPV, and HPIV in adults or transplant recipients. Premature infants receive the antibody palivizumab prophylactically to protect against RSV, but nothing is available for HMPV or the HPIVs. Palivizumab use is limited by the requirement for monthly reinjection due to short half-life.

The current disclosure provides new antibodies that are protective against one or more of human parainfluenza viruses (HPIV), respiratory syncytial virus (RSV), and human metapneumovirus (HMPV). Particular antibodies disclosed herein are protective against HPIV3. Other antibodies disclosed herein are protective against HPIV3 and HPIV1. Other antibodies disclosed herein are protective against RSV and HMPV. The newly disclosed antibodies can be used to protect against these various forms of respiratory viruses.

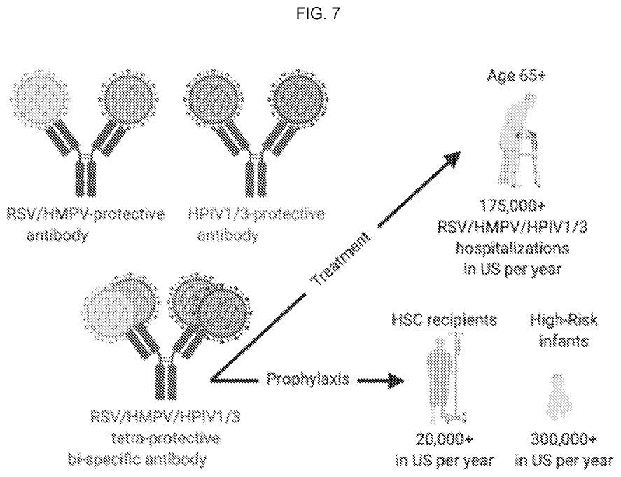

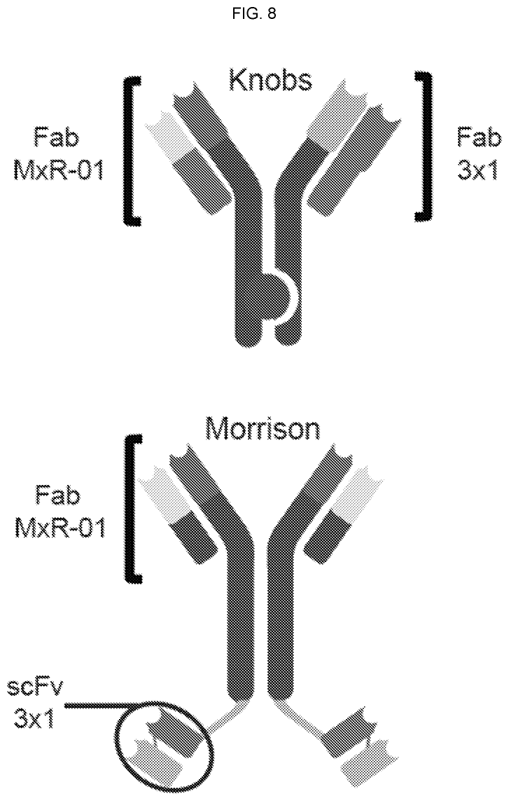

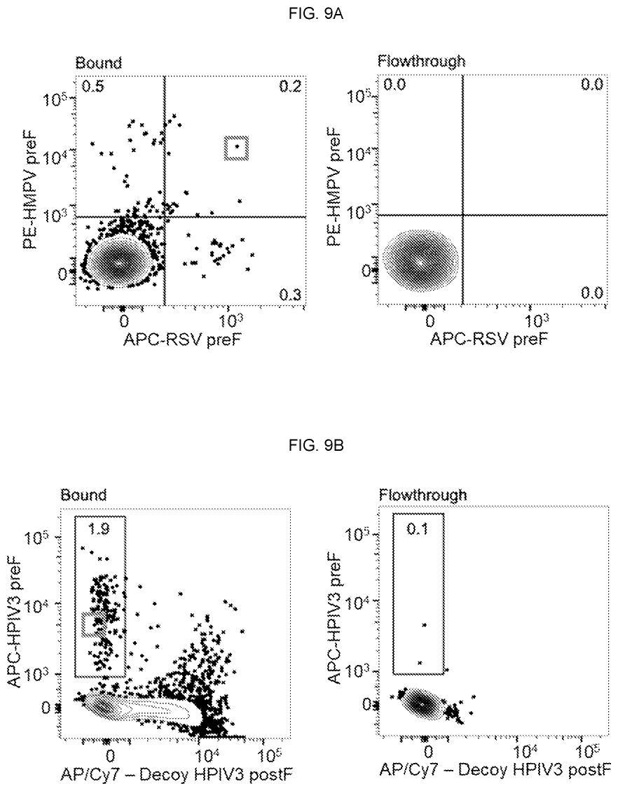

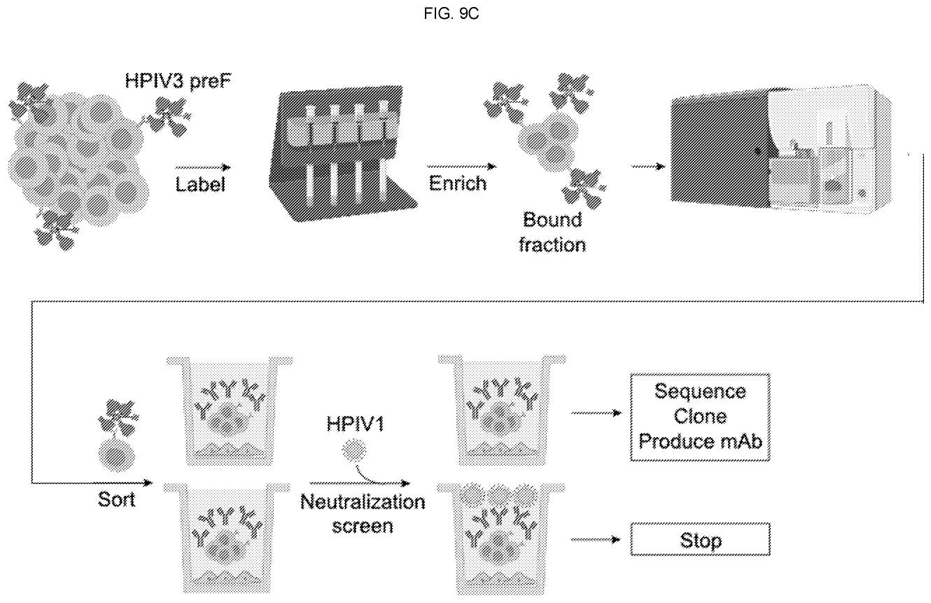

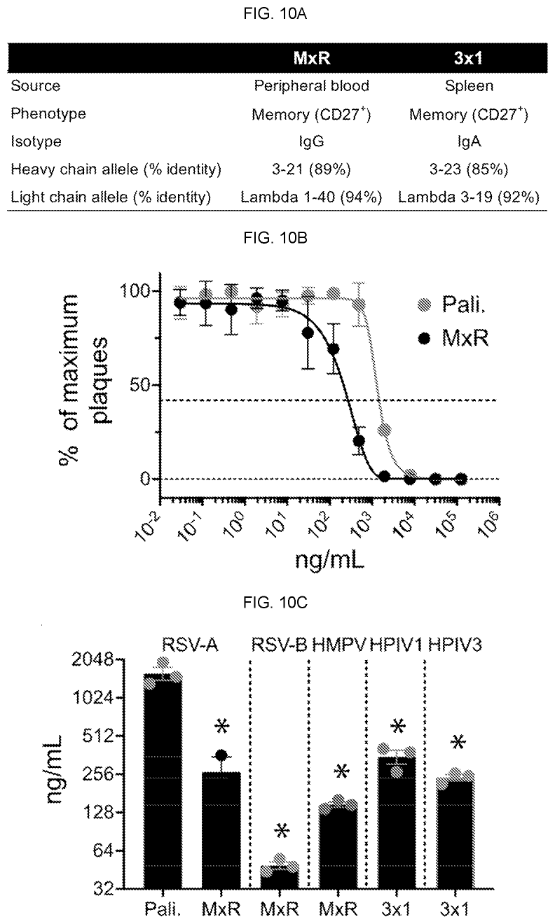

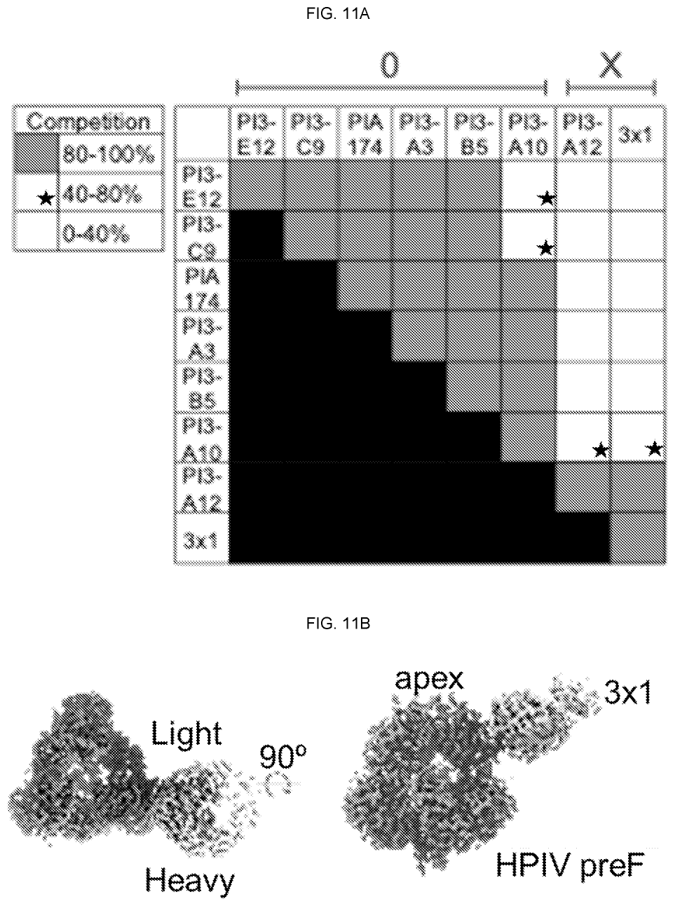

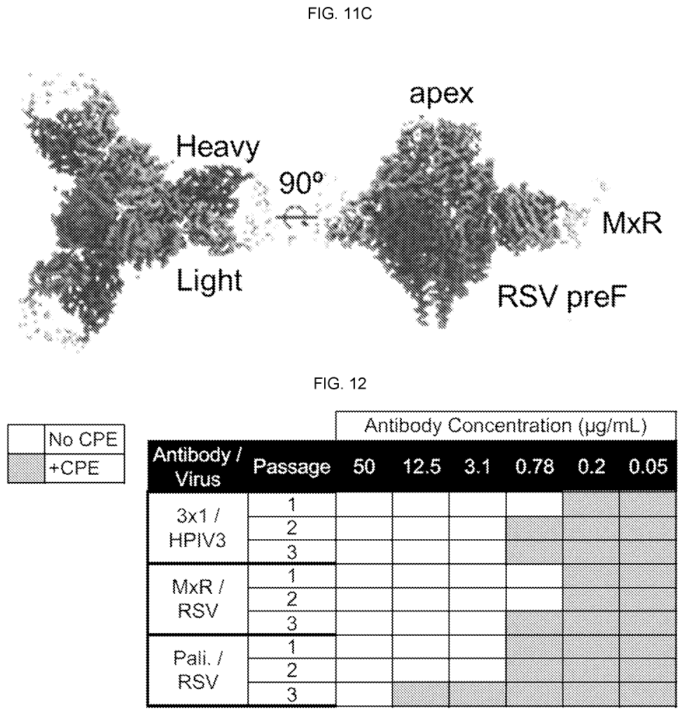

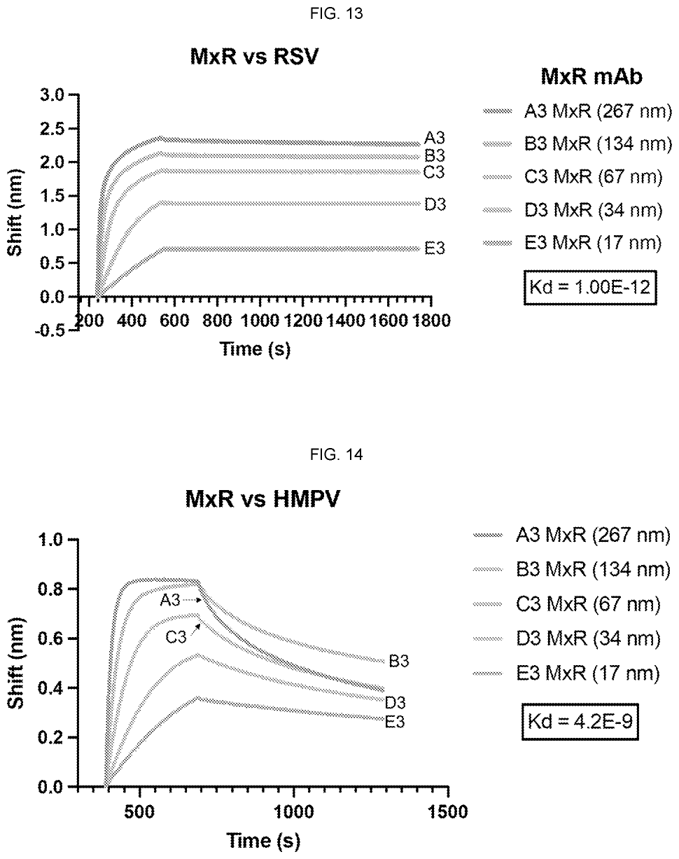

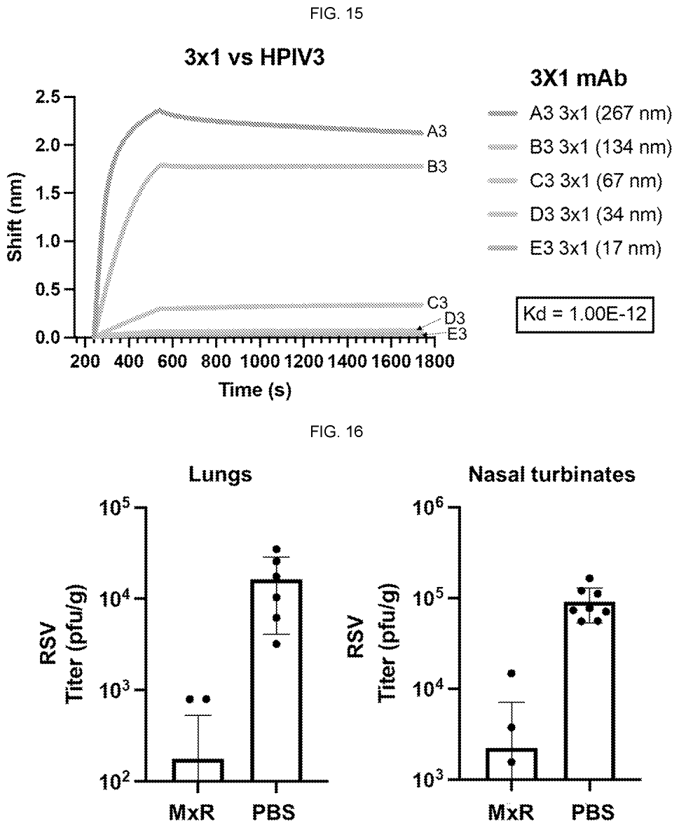

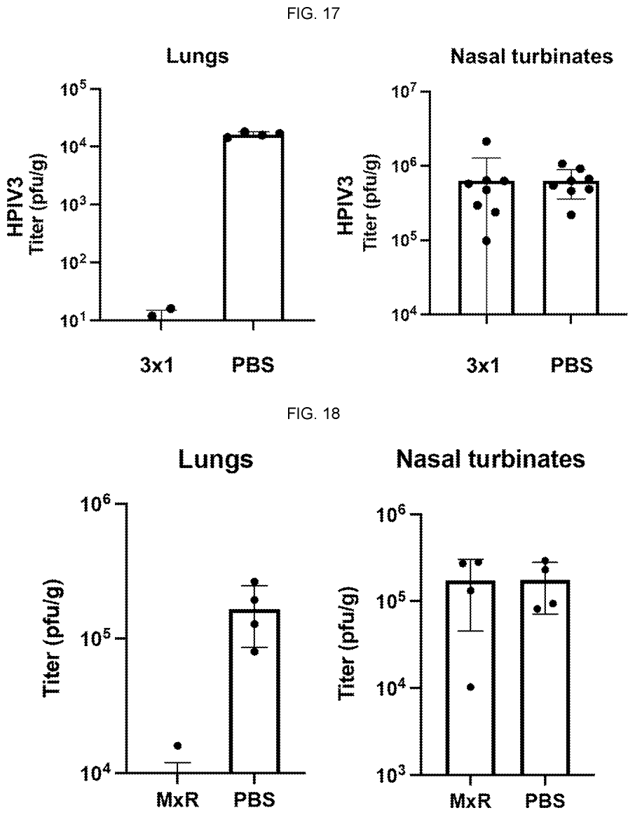

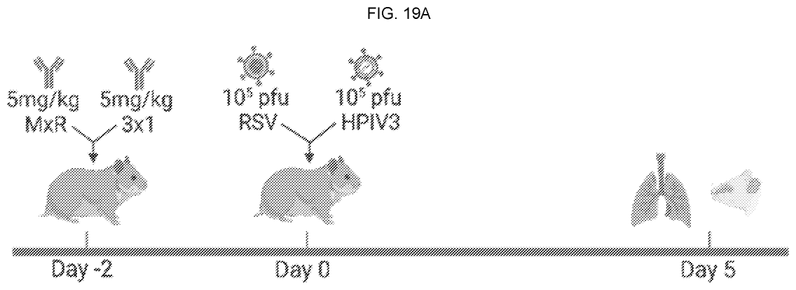

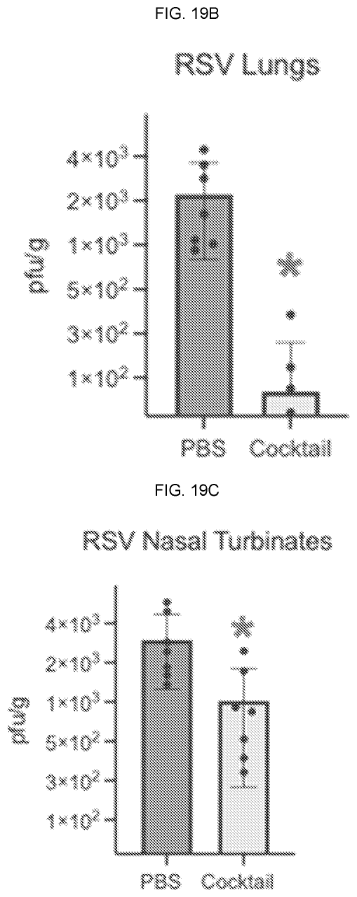

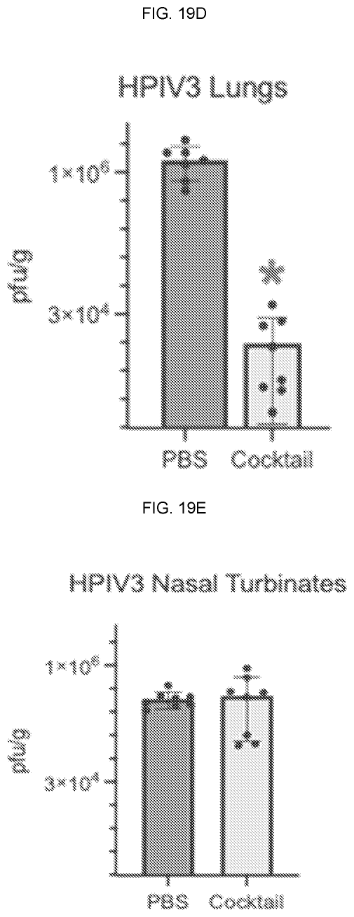

In particular embodiments, binding fragments from these newly-disclosed antibodies and others can be engineered into multi-specific formats that offer protection from multiple viruses at a time. That is, cross-neutralizing antibodies can be engineered into a single compound capable of protecting against numerous medically important respiratory viruses. One example, referred to herein as MRP3/1, includes a quad-protective, extended half-life bi-specific antibody engineered by tethering two cross-neutralizing mAbs (see, e.g., FIGS. 7 and 8). MRP3/1, simultaneously protects against HMPV, RSV, HPIV3, and HPIV1. This antibody is expected to have greater in vitro potency and breadth and a 4-6× longer half-life compared to palivizumab. Thus, MRP3/1 can replace palivizumab as the standard of care for RSV prophylaxis in premature infants with the added benefit of protecting against 3 additional viruses. Further, such engineered constructs can be used as immunoprophylaxis in HCT recipients during their 6-month period of vulnerability post-transplant or as a therapy for anyone hospitalized with any of these infections.

Aspects of the current disclosure are now described in more supporting detail as follows: (i) Antibodies and Engineered Binding Molecules; (ii) Formulations; (iii) Methods of Use; (iv) Exemplary Embodiments; (v) Experimental Examples; and (vi) Closing Paragraphs. These headings are provided for organizational purposes only and do not limit the scope or interpretation of the disclosure.

(i) Antibodies and Engineered Binding Molecules. Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Software programs and bioinformatical tools, such as ABodyBuilder and Paratome can also be used to determine CDR sequences. Additionally, delineation of a CDR can be according to X-ray crystallography.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g. B-cell receptors); and B-cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

As indicated, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

In particular embodiments, an epitope denotes the binding site on a viral peptide bound by a corresponding variable region of an antibody. The variable region either binds to a linear epitope, (e.g., an epitope including a stretch of 5 to 12 consecutive amino acids), or the variable region binds to a three-dimensional structure formed by the spatial arrangement of several short stretches of the protein target. Three-dimensional epitopes recognized by a variable region, e.g. by the epitope recognition site or paratope of an antibody or antibody fragment, can be thought of as three-dimensional surface features of an epitope molecule. These features fit precisely (in)to the corresponding binding site of the variable region and thereby binding between the variable region and its target protein (more generally, antigen) is facilitated. In particular embodiments, an epitope can be considered to have two levels: (i) the “covered patch” which can be thought of as the shadow an antibody variable region would cast on the antigen to which it binds; and (ii) the individual participating side chains and backbone residues that facilitate binding. Binding is then due to the aggregate of ionic interactions, hydrogen bonds, and hydrophobic interactions.

Epitopes of the currently disclosed antibodies (that is, epitopes to which the antibodies bind) are found on a virus selected from HPIV3, HPIV1, RSV, and/or HMPV. In particular embodiments, the epitope is located within a viral F protein, for example in its prefusion state.

In particular embodiments, “bind” means that the variable region associates with its target epitope with a dissociation constant (Kd or KD) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the variable region does not bind to other biomolecules present (e.g., it binds to other biomolecules with a dissociation constant (Kd) of 10⁻⁴ M or more, in particular embodiments of from 10⁻⁴ M to 1 M).

In particular embodiments, Kd can be characterized using BIAcore. For example, in particular embodiments, Kd can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 μM) before injection at a flow rate of 5 μl/minute to achieve 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 μl/min. Association rates (kₒₙ) and dissociation rates (k_{off}) can be calculated using a simple one-to-one Langmuir binding model (BIACORE® Evaluation Software version 3.2) by simultaneously fitting the association and dissociation sensorgrams. The equilibrium dissociation constant (Kd) can be calculated as the ratio k_{off}/kₒₙ. See, e.g., Chen et al., J. Mol. Biol. 293:865-881, 1999. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human or humanized antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup Ill as in Kabat et al. (supra).

A “humanized” antibody refers to a chimeric antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498,1991 (describing “resurfacing”); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

In particular embodiments, mAb PI3-E12 disclosed herein has a CDRH1 including GFTFSDHY (SEQ ID NO: 1); a CDRH2 including ISSSGSNT (SEQ ID NO: 2); a CDRH3 including ARAKWGTMGRGAPPTIYDH (SEQ ID NO: 3); a CDRL1 including QSLLQSNGNNY (SEQ ID NO: 4); a CDRL2 including LGS; and a CDRL3 including MQALQTPLT (SEQ ID NO: 5).

In particular embodiments, PI3-E12 has a heavy chain sequence including QVQLLESGGKLVKPGGSLRLSCAASGFTFSDHYMIWIRQAPGKGLEWISYISSSGSNTIYADSL MGRFTISRDNAKNSLYLQMNSLRTEDTAVYYCARAKWGTMGRGAPPTIYDHWGQGTLVTVSS (SEQ ID NO: 166) and a light chain sequence including DIVMTQSPLSLPVTPGEPASISCRSSQSLLQSNGNNYLEWYLQKPGQSPQLLIYLGSNRASGVP DRFSGSGSGTDFTLKISRVEAEDAGVYYCMQALQTPLTFGGGTKVEIK (SEQ ID NO: 167).

In particular embodiments, the PI3-E12 antibody includes a variable heavy chain sequence encoded by:

(SEQ ID NO: 6)
CAGGTGCAGCTGTTGGAGTCTGGGGGAAAGTTGGTCAAGCCTGGAGGGT
CCCTGAGACTCTCCTGTGCAGCCTCTGGATTCACCTTCAGTGACCACTA
CATGATCTGGATCCGCCAGGCTCCCGGGAAGGGGCTGGAGTGGATTTCA
TACATAAGTAGTAGTGGTAGTAACACAATCTACGCAGACTCTTTGATGG
GCCGATTCACCATCTCCAGAGACAACGCCAAGAACTCTCTGTATCTACA
AATGAACAGCCTGAGGACCGAGGACACGGCCGTTTATTACTGTGCGAGA
GCAAAGTGGGGTACTATGGGTCGGGGAGCACCCCCGACAATTTATGACC
ACTGGGGCCAGGGAACCCTGGTCACCGTCTCCTCA

and a variable kappa light chain sequence encoded by:

(SEQ ID NO: 7)
GATATTGTGATGACTCAGTCTCCACTCTCCCTGCCCGTCACCCCTGGAG
AGCCGGCCTCCATCTCCTGTAGGTCTAGTCAGAGCCTCCTGCAAAGTAA
TGGAAACAATTATTTGGAGTGGTACCTGCAGAAGCCAGGGCAGTCTCCA
CAACTCCTGATCTATTTGGGTTCCAATCGGGCCTCCGGGGTCCCTGACA
GGTTCAGTGGCAGTGGATCAGGCACAGATTTTACACTGAAGATCAGCAG
AGTGGAGGCTGAGGATGCTGGGGTTTATTACTGCATGCAAGCTCTACAA
ACTCCGCTCACTTTCGGCGGAGGGACCAAGGTGGAGATCAAAC.

In particular embodiments, mAb PI3-A3 disclosed herein has a CDRH1 including GFTFSNYW (SEQ ID NO: 8); a CDRH2 including VKEEGSEK (SEQ ID NO: 9); a CDRH3 including AGEVKSGWFGRYFDS (SEQ ID NO: 10); a CDRL1 including QSVGSW (SEQ ID NO: 11); a CDRL2 including KTS; and a CDRL3 including QQYSSFPYT (SEQ ID NO: 12).

In particular embodiments mAb PI3-A3 has a heavy chain sequence including EVQLVESGGGLVQPGGSLRLSCTASGFTFSNYWMSWVRQAPGKGLEWVANVKEEGSEKHY VDSVKGRFTISRDNAKNSVYLQMSSLRAEDTAVYYCAGEVKSGWFGRYFDSWGQGTLVTVSS (SEQ ID NO: 168) and a light chain sequence including DIQMTQSPSTLSASVGDRVTINCRASQSVGSWLAWYQQKPGKAPKLLMYKTSTLQRGVPSRF SGSGSGTEFTLTISSLQPDDFAAYYCQQYSSFPYTFGQGTKLEIK (SEQ ID NO: 169).

In particular embodiments, the PI3-A3 antibody includes a variable heavy chain sequence encoded by:

(SEQ ID NO: 13)
GAGGTGCAGCTGGTGGAGTCTGGGGGAGGCTTGGTCCAGCCTGGGGGGT
CCCTGAGACTCTCCTGTACAGCCTCTGGATTCACCTTTAGCAATTATTG
GATGAGCTGGGTCCGCCAGGCTCCAGGGAAGGGGCTGGAGTGGGTGGCC
AATGTGAAGGAAGAAGGAAGTGAGAAACACTATGTAGACTCTGTGAAGG
GCCGATTCACCATCTCCAGAGACAACGCCAAGAACTCAGTGTATCTGCA
GATGAGCAGCCTGAGAGCCGAGGACACGGCTGTCTATTACTGTGCGGGA
GAGGTTAAGAGTGGCTGGTTCGGTCGGTACTTTGACTCCTGGGGCCAGG
GAACCCTGGTCACCGTCTCCTCAG

and a variable kappa light chain sequence encoded by:

(SEQ ID NO: 14)
GACATCCAGATGACCCAGTCTCCTTCCACCCTGTCTGCATCTGTTGGAG
ACAGAGTCACCATCAATTGCCGGGCCAGTCAGAGTGTTGGTAGCTGGTT
GGCCTGGTATCAGCAGAAACCAGGGAAAGCCCCTAAGCTCCTGATGTAT
AAGACATCTACTTTACAAAGAGGGGTCCCATCAAGGTTCAGCGGCAGTG
GATCTGGGACAGAATTCACTCTCACCATCAGCAGCCTGCAGCCTGATGA
TTTTGCAGCTTATTACTGCCAACAGTATAGTAGTTTTCCGTACACTTTT
GGCCAGGGGACCAAGCTGGAGATCAAAC.

In particular embodiments, mAb PI3-B5 disclosed herein has a CDRH1 including GYNFTNYW (SEQ ID NO: 15); a CDRH2 including IYPADSDT (SEQ ID NO: 16); a CDRH3 including ARPSTRWFVPGGMDV (SEQ ID NO: 17); a CDRL1 including QSIGAW (SEQ ID NO: 18); a CDRL2 including KAS; and a CDRL3 including QQHSSYPST (SEQ ID NO: 19).

In particular embodiments, mAb PI3-B5 has a heavy chain sequence including EVQLVQSGAEVKKPGESLRISCKGSGYNFTNYWIAWVRQMPGKGLEWMGIIYPADSDTRYSP SFQGQVTISADKSITTAYLQWSSLKASDTAIYYCARPSTRWFVPGGMDVWGQGTTVIVSS (SEQ ID NO: 170) and a light chain sequence including DIQMTQSPSTLSASVGDRVTISCRATQSIGAWLAWYQQKPGEPPKLLIYKASTLESGVPSRFSG SGSGTEFTLTISSLQPDDSATYYCQQHSSYPSTFGGGTKVEIK (SEQ ID NO: 171).

In particular embodiments, the PI3-B5 antibody includes a variable heavy chain sequence encoded by:

(SEQ ID NO: 20)
GAGGTGCAGCTGGTGCAGTCTGGAGCAGAGGTGAAAAAGCCCGGGGAGT
CTCTGAGGATCTCCTGTAAGGGTTCTGGATACAACTTTACCAACTACTG
GATCGCCTGGGTGCGCCAGATGCCCGGGAAAGGCCTGGAGTGGATGGGG
ATCATCTATCCTGCTGACTCGGATACCAGATACAGCCCGTCCTTCCAAG
GCCAAGTCACCATCTCAGCCGACAAGTCCATCACCACCGCCTACCTGCA
GTGGAGCAGCCTGAAGGCCTCGGACACCGCCATATACTACTGTGCGAGA
CCGAGTACTAGGTGGTTCGTCCCTGGCGGTATGGACGTCTGGGGCCAAG
GCACCACGGTCATCGTCTCCTCA

and a variable kappa light chain sequence encoded by:

(SEQ ID NO: 21)
GACATCCAGATGACCCAGTCTCCTTCCACCCTGTCTGCTTCTGTAGGAG
ACAGAGTCACCATTTCTTGCCGGGCCACTCAGAGTATTGGTGCCTGGTT
GGCCTGGTATCAGCAGAAACCAGGGGAACCCCCTAAGCTCCTGATCTAT
AAGGCGTCTACTTTAGAGAGTGGGGTCCCATCAAGGTTCAGCGGCAGTG
GATCTGGGACAGAATTCACTCTCACCATCAGCAGCCTGCAGCCTGATGA
TTCTGCAACTTATTACTGCCAACAGCATAGTAGTTATCCTTCTACTTTC
GGCGGAGGGACCAAGGTGGAGATCAAAC.

In particular embodiments, mAb PI3-A10 disclosed herein has a CDRH1 including GFNFNNYG (SEQ ID NO: 22); a CDRH2 including VSFDGSNR (SEQ ID NO: 23); a CDRH3 including SKSKYSDFWSEI (SEQ ID NO: 24); a CDRL1 including QNVMRY (SEQ ID NO: 25); a CDRL2 including DAS; and a CDRL3 including QQRTNHRFS (SEQ ID NO: 26).

In particular embodiments, mAb PI3-A10 has a heavy chain sequence including QVQLVESGGGVVRPGRSLRLSCVASGFNFNNYGLQWIRQAPGKGLEWVAGVSFDGSNRYYA DSVKGRVTISRDDSKNTLYLEMNSLRAEDTGIYYCSKSKYSDFWSEIWGQGTLVTVSS (SEQ ID NO: 172) and a light chain sequence including EIVLTQSPATLSLSPGERATLSCRASQNVMRYLAWYQQRPGQAPRLLFYDASSRATGIPARFT ASGSGTDFTLTISGLEPGDFAVYYCQQRTNHRFSFGPGTKVDIK (SEQ ID NO: 173).

In particular embodiments, the PI3-A10 antibody includes a variable heavy chain sequence encoded by:

(SEQ ID NO: 27)
CAGGTGCAGCTGGTGGAGTCTGGGGGAGGCGTGGTCCGGCCTGGGAGGT
CCCTGAGACTCTCCTGTGTAGCCTCTGGATTCAACTTCAATAACTATGG
GCTGCAGTGGATCCGCCAGGCTCCAGGCAAGGGGCTGGAGTGGGTGGCA
GGTGTCTCGTTTGATGGGAGTAATAGATATTATGCAGACTCCGTGAAGG
GCCGAGTCACCATATCCAGAGACGATTCCAAGAACACCCTGTATCTAGA
AATGAACAGCCTGAGAGCTGAGGACACAGGAATATATTACTGTTCGAAG
TCCAAGTACTCCGACTTTTGGAGCGAAATATGGGGCCAGGGAACCCTGG
TCACCGTCTCCTCAG

and a variable kappa light chain sequence encoded by:

(SEQ ID NO: 28)
GAAATTGTGTTGACACAGTCTCCAGCCACCCTGTCTTTGTCTCCAGGGG
AAAGAGCCACCCTCTCCTGCAGGGCCAGTCAGAATGTTATGAGGTACTT
AGCCTGGTACCAACAGAGACCTGGCCAGGCTCCCAGACTCCTCTTCTAT
GATGCATCCAGCCGGGCCACTGGCATCCCAGCCCGGTTCACTGCCAGTG
GGTCTGGGACAGACTTCACTCTCACCATCAGCGGCCTCGAGCCTGGAGA
TTTTGCAGTTTATTACTGTCAGCAGCGTACCAACCATAGATTCTCTTTC
GGCCCTGGGACCAAGGTGGATATCAAAC.

In particular embodiments, mAb PI3-A12 disclosed herein has a CDRH1 including GDSVKSDDFY (SEQ ID NO: 29); a CDRH2 including IYYGGRT (SEQ ID NO: 30); a CDRH3 including VRVEGLLWFGELFDY (SEQ ID NO: 31); a CDRL1 including NSNIGNNF (SEQ ID NO: 32); a CDRL2 including KDY; and a CDRL3 including AAWQDGLSGPL (SEQ ID NO: 33).

In particular embodiments, mAb PI3-A12 has a heavy chain sequence including QVQLQESGPGLVKPSETLSLTCTVSGDSVKSDDFYWSWIRQPPGKGLEWIGFIYYGGRTYYNP SLSGRGTISVDTSKNHFFLELTSVTAADTAVYYCVRVEGLLWFGELFDYWGQGTLVTVSS (SEQ ID NO: 174) and a light chain sequence including QSVLTQPPSASGTPGQRVTISCSGSNSNIGNNFVYWYQQVPGSAPKVVIYKDYQRPSGVPDR FSASKSGTSASLTISGLRSDDEAHYYCAAWQDGLSGPLFGGGTKLTVL (SEQ ID NO: 175).

In particular embodiments, the PI3-A12 antibody includes a variable heavy chain sequence encoded by:

(SEQ ID NO: 34)
CAGGTGCAGCTGCAGGAGTCGGGCCCAGGACTGGTGAAGCCTTCAGAGA
CCCTGTCCCTCACTTGCACTGTCTCTGGTGACTCCGTCAAGAGTGATGA
TTTCTACTGGAGTTGGATCCGGCAGCCCCCAGGGAAGGGCCTGGAGTGG
ATTGGCTTCATCTATTACGGTGGCAGAACTTACTACAACCCGTCCCTCA
GTGGTCGAGGAACCATTTCAGTGGACACGTCCAAGAACCACTTCTTCCT
GGAGCTGACCTCTGTGACTGCCGCAGACACGGCCGTATACTACTGTGTC
AGGGTCGAAGGATTACTGTGGTTCGGGGAGTTATTCGACTACTGGGGCC
AGGGAACCCTGGTCACCGTCTCCTCAG

and a variable lambda light chain sequence encoded by:

(SEQ ID NO: 35)
CAGTCTGTGCTGACGCAGCCACCCTCAGCGTCTGGGACCCCCGGGCAGA
GGGTCACCATCTCTTGTTCTGGAAGCAACTCCAACATCGGAAATAATTT
TGTCTACTGGTACCAACAAGTCCCAGGATCGGCCCCCAAAGTCGTCATT
TACAAGGATTATCAGCGCCCCTCAGGGGTCCCTGACCGATTCTCTGCCT
CCAAGTCTGGCACCTCAGCCTCCCTGACCATCAGCGGGCTCCGGTCCGA
CGATGAGGCCCATTATTACTGTGCAGCATGGCAGGACGGTCTGAGTGGG
CCGTTATTTGGCGGAGGGACCAAGCTGACCGTCCTAG.

In particular embodiments, mAb 3×1 disclosed herein has a CDRH1 including GFTFSSFG (SEQ ID NO: 36); a CDRH2 including ISHSAGFL (SEQ ID NO: 37); a CDRH3 including AKRLAGLPDLEWLLYPNFLDH (SEQ ID NO: 38); a CDRL1 including ILRTYY (SEQ ID NO: 39); a CDRL2 including GKN; and a CDRL3 including SSRDRSGNHVL (SEQ ID NO: 40).

In particular embodiments, mAb 3×1 has a heavy chain sequence including EVQLLESGGGLVQPGGSLRLSCAASGFTFSSFGMSWVRQSPGKGLEWVADISHSAGFLNYAD SVKGRFTVSRDNSKSTLHLQMKSLRAEDTAVYYCAKRLAGLPDLEWLLYPNFLDHWGQGTLV TVSS (SEQ ID NO: 176) and a light chain sequence including SSELTQDPAVSVALGQTVRITCQGDILRTYYVSWYQQKPGQAPLLVIYGKNNRPSVIPDRFSGS TSGDTASLTITGAQAEDEAEYYCSSRDRSGNHVLFGGGTKLTVL (SEQ ID NO: 177).

In particular embodiments, the 3×1 xnAb antibody includes a variable heavy chain sequence encoded by:

(SEQ ID NO: 41)
GAGGTGCAGCTGTTGGAGTCTGGGGGAGGCTTGGTACAGCCTGGTGGGT
CCCTGAGACTGTCTTGTGCGGCCTCAGGATTCACCTTTAGCAGCTTTGG
CATGAGCTGGGTCCGCCAATCTCCAGGAAAGGGGCTGGAGTGGGTCGCA
GATATAAGCCATAGTGCTGGCTTCTTAAACTACGCAGACTCCGTGAAGG
GCCGGTTCACCGTCTCCAGAGACAATTCTAAGAGCACGCTGCATCTCCA
AATGAAGAGCCTGAGAGCCGAGGACACGGCCGTATATTACTGTGCGAAA
AGACTTGCCGGATTACCAGATTTGGAGTGGTTACTTTACCCTAACTTCT
TAGACCACTGGGGCCAGGGAACCCTGGTCACCGTCTCCTCAG

and a variable lambda light chain sequence encoded by:

(SEQ ID NO: 42)
TCTTCTGAGCTGACTCAGGACCCTGCTGTGTCTGTGGCCTTGGGACAGA
CAGTCAGGATCACATGCCAAGGAGACATACTGAGAACCTATTATGTAAG
CTGGTACCAGCAGAAACCAGGACAGGCCCCGCTACTTGTCATCTATGGT
AAAAACAACCGACCCTCAGTGATCCCAGACCGATTCTCTGGCTCCACCT
CAGGAGACACAGCTTCCTTGACCATCACTGGGGCTCAGGCGGAAGATGA
GGCTGAGTATTATTGTAGCTCTCGGGACAGGAGTGGAAACCATGTGCTA
TTCGGCGGAGGGACCAAGCTGACCGTCCTAG.

In particular embodiments, mAb MxR-B11 disclosed herein (also referred to as MxR-01) has a CDRH1 including GFPFSSYK (SEQ ID NO: 43); a CDRH2 including ISASGSYI (SEQ ID NO: 44); a CDRH3 including ARDGGRELSPFEK (SEQ ID NO: 45); a CDRL1 including NSNIGTGYD (SEQ ID NO: 46); a CDRL2 including DNN; and a CDRL3 including QSYDKSLGGWV (SEQ ID NO: 47).

In particular embodiments, MxR-B11 (MxR-01) includes a variable heavy chain having the sequence as set forth in

EVQVVESGGGLVKPGGSLRLSCAASGFPFSSYKMDWVRQAPGKGLEWVSSISASGSYINYAD SVKGRFTISRDNAKNSLYLQMKSLRADDTAVYFCARDGGRELSPFEKWGQGILVTVSS (SEQ ID NO: 178) and a variable light chain having the sequence as set forth in

### QSVLTQPPSVSGAPGQRVTISCTGTNSNIGTGYDVHWYQQLPGTAPKVVLFDNNNRPSGVPD RFSGSKSGTSAALAITGLQAEDEAVYYCQSYDKSLGGWVFGGGTKLTVL (SEQ ID NO: 179).

In particular embodiments, the MxR-B11 antibody includes a variable heavy chain encoded by the sequence:

(SEQ ID NO: 48)
GAGGTGCAGGTGGTGGAGTCTGGGGGAGGCCTGGTCAAGCCTGGGGGGT
CCCTGAGACTCTCCTGTGCAGCCTCTGGATTCCCCTTCAGTTCTTATAA
GATGGACTGGGTCCGCCAGGCTCCAGGGAAGGGGCTGGAGTGGGTCTCG
TCCATCAGTGCTAGTGGAAGTTACATAAACTATGCAGACTCAGTGAAGG
GCCGATTCACCATCTCCAGAGACAACGCCAAGAACTCACTGTATCTGCA
AATGAAAAGCCTGAGAGCCGACGACACGGCTGTATATTTTTGTGCGAGA
GACGGCGGAAGAGAACTGAGCCCCTTTGAAAAGTGGGGCCAGGGAATCC
TGGTCACCGTCTCCTCAG

and a variable light chain encoded by the sequence:

(SEQ ID NO: 49)
CAGTCTGTGCTGACGCAGCCGCCCTCAGTGTCTGGGGCCCCAGGACAGA
GGGTCACCATCTCCTGCACTGGGACCAACTCCAACATCGGGACAGGTTA
TGATGTACACTGGTACCAGCAGCTTCCGGGAACAGCCCCCAAAGTCGTC
CTCTTTGATAACAACAATCGGCCCTCAGGGGTCCCTGACCGATTCTCTG
GCTCCAAGTCTGGCACTTCAGCCGCCCTGGCCATCACTGGCCTCCAGGC
TGAGGATGAGGCTGTTTATTACTGCCAGTCCTATGACAAGAGCCTGGGT
GGTTGGGTGTTCGGCGGAGGGACCAAGCTGACCGTCCTAG.

In particular embodiments, mAb MxR-D10 disclosed herein (also referred to as MxR-02) has a CDRH1 including GFIFSNYD (SEQ ID NO: 50); a CDRH2 including ITGGSSFI (SEQ ID NO: 51); a CDRH3 including ARDGGRQLSPCEH (SEQ ID NO: 52); a CDRL1 including SSNIGAGYD (SEQ ID NO: 53); a CDRL2 including DNN; and a CDRL3 including QSYDRGLSGWA (SEQ ID NO: 54).

In particular embodiments, mAb MxR-D10 (MxR-02) includes a variable heavy chain having the sequence

EVQVVESGGGLVKPGGSLRLSCTASGFIFSNYDMNWVRQAPGKGLEWVASITGGSSFINYAD SVKGRFTISRDNAKKSLYLQMNSLRAEDTAVYYCARDGGRQLSPCEHWGQGTLVTVSS (SEQ ID NO: 180) and a variable light chain having the sequence

### QSVLTQPPSVSGSPGQRVTISCTGGSSNIGAGYDVHWYQQLPGSAPKLLMYDSNNRPSGVPD RFSGSKSGTSASLAITGLQAEDEADYYCQSYDRGLSGWAFGGGTKLTVL (SEQ ID NO: 181).

In particular embodiments, the MxR-D10 antibody includes a variable heavy chain encoded by the sequence:

(SEQ ID NO: 55)
GAGGTGCAGGTGGTGGAGTCGGGGGGAGGCCTGGTCAAGCCTGGGGGGT
CCCTGAGACTCTCCTGTACAGCCTCTGGATTCATATTCAGTAATTATGA
CATGAACTGGGTCCGCCAGGCTCCAGGGAAGGGCCTGGAGTGGGTCGCC
TCCATTACTGGTGGTAGTAGTTTCATAAATTACGCAGACTCAGTGAAGG
GCCGATTCACCATCTCCAGAGACAACGCCAAGAAGTCACTGTATCTGCA
AATGAACAGCCTCAGAGCCGAGGACACGGCTGTCTATTACTGTGCGAGA
GATGGCGGGAGACAGTTGAGTCCGTGTGAACATTGGGGCCAGGGAACCC
TGGTCACCGTCTCCTCAG

and a variable light chain encoded by the sequence:

(SEQ ID NO: 56)
CAGTCTGTGCTGACGCAGCCGCCCTCAGTGTCTGGGTCCCCAGGGCAGA
GGGTCACCATCTCCTGCACTGGGGGCAGCTCCAACATCGGGGCAGGTTA
TGATGTACACTGGTACCAACAACTTCCAGGATCAGCCCCCAAACTCCTC
ATGTATGATAGTAATAATCGACCCTCAGGGGTCCCTGACCGATTCTCTG
GCTCCAAGTCTGGCACCTCAGCCTCCCTGGCCATCACTGGGCTCCAGGC
TGAGGATGAGGCTGATTATTACTGCCAGTCCTATGACAGGGGCCTGAGT
GGGTGGGCGTTCGGCGGAGGGACCAAGCTGACCGTCCTGG.

As indicated, antibodies disclosed herein can be utilized to prepare various forms of relevant binding fragment molecules. For example, particular embodiments can include binding fragments of an antibody, e.g., Fv, Fab, Fab′, F(ab′)2, and single chain Fv fragments (scFvs) or any biologically effective fragments of an immunoglobulin that bind specifically to an epitope described herein.

In particular embodiments, an antibody fragment is used. An “antibody fragment” denotes a portion of a complete or full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Additional examples of antibody-based binding fragment formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

Variants of antibodies described herein are also included. Variants of antibodies can include those having one or more conservative amino acid substitutions or one or more non-conservative substitutions that do not adversely affect the binding of the protein.

In particular embodiments, a conservative amino acid substitution may not substantially change the structural characteristics of the reference sequence (e.g., a replacement amino acid should not tend to break a helix that occurs in the reference sequence or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a V_{L} region can be derived from or based on a disclosed V_{L} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the disclosed V_{L}. An insertion, deletion or substitution may be anywhere in the V_{L}region, including at the amino- or carboxy-terminus or both ends of this region, provided that each CDR includes zero changes or at most one, two, or three changes and provided an antibody including the modified V_{L} region can still specifically bind its target epitope with an affinity similar to the wild type binding fragment.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} disclosed herein. An insertion, deletion or substitution may be anywhere in the V_{H} region, including at the amino- or carboxy-terminus or both ends of this region, provided that each CDR includes zero changes or at most one, two, or three changes and provided an antibody including the modified V_{H} region can still specifically bind its target epitope with an affinity similar to the wild type binding fragment.

In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to an antibody sequence disclosed herein. In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a light chain variable region (V_{L}) and/or to a heavy chain variable region (V_{H}), or both, wherein each CDR includes zero changes or at most one, two, or three changes, from the reference antibody disclosed herein or fragment or derivative thereof that specifically binds to the target viral epitope.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live. While the disclosure below describes these modifications in terms of their application to antibodies, when applicable to another particular binding fragment format (e.g., an scFv, bispecific antibodies), the modifications can also be applied to these other formats.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations.

In particular embodiments, Fc polypeptide amino acid alterations can include M252Y, S254T, T256E, M428L, and/or N434S and can be used together, separately or in any combination. For example, the introduction of the “YTE” mutation (M252Y, S254T, and T256E) into the Fc region of an IgG can extend its half-life 4-fold from 3-weeks to over 3-months in non-human primates, potentially allowing the administration of a single dose to span the entire respiratory virus season. More detail regarding the YTE mutation can be found in Acqua et al., J. Immunol. 169(9) 5171-5180 (2002). M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein.

In particular embodiments, antibodies disclosed herein are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/I of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according the methods described herein.

Anti-viral bispecific antibodies bind at least two epitopes wherein at least one of the epitopes is located on HPIV3, HPIV1, RSV or HMPV. Anti-viral trispecific antibodies bind at least 3 epitopes, wherein at least one of the epitopes is located on HPIV3, HPIV1, RSV or HMPV, and so on. Bispecific antibodies can be prepared as full-length antibodies or antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody. In particular embodiments, a bispecific antibody can be in the form of a Bispecific T-cell Engaging (BiTE®) antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

scFv dimers or diabodies may be used, rather than whole antibodies. Diabodies and scFv can be constructed without an Fc region, using only variable domains (usually including the variable domain components from both light and heavy chains of the source antibody), potentially reducing the effects of anti-idiotypic reaction. Other forms of bispecific antibodies include the single chain “Janusins” described in Traunecker et al. (Embo Journal, 10, 3655-3659, 1991).

Bispecific antibodies with extended half-lives are described in, for example, U.S. Pat. No. 8,921,528 and US Patent Publication No. 2014/0308285.

Methods for making bispecific antibodies are known in the art. For example, traditional production of full-length bispecific antibodies is based on the co-expression of two immunoglobulin heavy chain-light chain pairs, where the two chains have different specificities (see, for example, Millstein et al. Nature 305:37-39, 1983). Similar procedures are disclosed in, for example, WO 1993/008829, Traunecker et al., EMBO J. 10:3655-3659, 1991 and Holliger & Winter, Current Opinion Biotechnol. 4, 446-449 (1993).

In particular embodiments, bispecific antibodies can be prepared using chemical linkage. For example, Brennan et al. (Science 229: 81, 1985) describes a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody.

In particular embodiments, bispecific antibodies can be prepared using knobs-into holes techniques. Knobs-into-holes refers to forcing the pairing of two different antibody heavy chains by introducing mutations into the CH3 domains to modify the contact interface. On one chain bulky amino acids are replaced by amino acids with short side chains to create a ‘hole’. Conversely, amino acids with large side chains were introduced into the other CH3 domain, to create a ‘knob’. By coexpressing these two heavy chains (and two identical light chains, which have to be appropriate for both heavy chains), high yields of heterodimer formation (‘knob-hole’) versus homodimer formation (‘hole-hole’ or ‘knob-knob’) is observed (Ridgway, J. B., Protein Eng. 9 (1996) 617-621; and WO 96/027011).

In particular embodiments, the ‘knob’ and/or the ‘hole’ may exist in the original polypeptide or may be introduced synthetically (e.g. by altering nucleic acid encoding the polypeptide). To synthetically introduce a knob and/or hole, the nucleic acid encoding the original amino acid residue (or other non-amino acid groups such as, for example carbohydrate groups) in the interface of the polypeptide is replaced with DNA encoding at least one import amino acid residue, wherein the interface refers to amino acid residues in contact between a first heavy chain constant region and one or more amino acid residues (or other non-amino acid groups) in a second heavy chain constant region. The preferred import residues for the formation of a hole are amino acids with smaller side chain volumes than the original amino acid residue such as alanine (A), serine (S), threonine (T), valine (V), or glycine (G). The preferred import residues for the formation of a knob are amino acids with larger side chain volumes than the original amino acid residue such as tyrosine (Y), arginine (R), phenylalanine (F), or tryptophan (W). The percentage of heterodimer can be increased by remodeling the interaction surfaces of the two CH3 domains using a phage display approach and the introduction of a disulfide bridge to stabilize the heterodimers (Merchant A. M, et al., Nature Biotech 16 (1998) 677-681; Atwell, S., et al., J. Mol. Biol. 270 (1997) 26-35).

In particular embodiments, binding fragments disclosed herein can be used to create bi-tri-, quad-(or more) specific antibody constructs that bind a secondary virus. As used herein, a secondary virus is one that is not HPIV3, HPIV1, RSV or HMPV. In particular embodiments, the secondary virus is a respiratory virus. In particular embodiments, the secondary respiratory virus is selected from an adenovirus, a boca virus, a coronavirus, an enterovirus, an influenza virus, a metapneumovirus, a parainfluenza virus, and/or a rhinovirus. In particular embodiments, the respiratory virus includes: human adenovirus, human boca virus (HBoV), human coronavirus (HCoV, including SARS-CoV, MERS-CoV, coronavirus 229E, coronavirus OC43, coronavirus NL63, coronavirus HKU1, coronavirus NL, coronavirus NH), influenza (groups A and B), human parainfluenza virus (HPIV2 or 4), and/or human rhinovirus (HRV A-HRVC).

Exemplary binding fragments that can be used in an engineered format that binds a secondary virus include: 8C4, 5Hx-1, 5Hx-2, 5Hx-3, 5Hx-4, 5Hx-5, 5.100K-1, 5PB-1, 5Fb-1, and 1E11 to bind to adenovirus; EPR23305-44 to bind to coxsackie adenovirus; 47D11 antibody to bind to SARS-CoV and SARS-CoV-2; CR3022 to bind to SARS-CoV-2; CDC2-A2, G2, 5F9, FIB-H1, and JC57-13 to bind to MERS-CoV; 32D6 to bind to H1N1 influenza virus; CH65 to bind to H1 influenza virus; CR9114, MAb 22/1, MAb70/I, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, and PEG-1 to bind to influenza A virus; CR8033, CR8071, 113/2, 124/4, 128/2, 134/1, 146/1, 152/2, 160/1, 162/1, 195/3, 206/2, 238/4, and 280/2 to bind to influenza B virus; PAR2 (boca231/9F) to bind to HPIV2; or TCN-711 to bind to rhinovirus.

Exemplary binding fragments that can be used in an engineered format that binds a secondary virus such as MERS-CoV include: a heavy chain with three CDRs including the amino acid sequences EFTFNTYG (SEQ ID NO: 57), ISYDGTKK (SEQ ID NO: 58), and ARSGDSDAFDI (SEQ ID NO: 59) respectively and/or a light chain with three CDRs including the amino acid sequences ELGDKF (SEQ ID NO: 60), QDS, and QAWDSNSYV (SEQ ID NO: 61) respectively.

Exemplary binding fragments that can be used in an engineered format that binds a secondary virus such as MERS-CoV include: a heavy chain with three CDRs including the amino acid sequences GGTFGSYA (SEQ ID NO: 62), IDAANGNT (SEQ ID NO: 63), and ARDRWMTTRAFDI (SEQ ID NO: 64) respectively and/or a light chain with three CDRs including the amino acid sequences SSNIGSNY (SEQ ID NO: 65), RNN, and AAWDDSLRGPV (SEQ ID NO: 66) respectively.

Exemplary binding fragments that can be used in an engineered format that binds a secondary virus such as MERS-CoV include: a heavy chain with three CDRs including the amino acid sequences GGTFSSYA (SEQ ID NO: 135), IIPIFGKA (SEQ ID NO: 136), and ARDQGISANFKDAFDI (SEQ ID NO: 137) respectively and/or a light chain with three CDRs including the amino acid sequences ESVGSN (SEQ ID NO: 138), GAS, and QQYNNWPLT (SEQ ID NO: 139) respectively.

Exemplary binding fragments that can be used in an engineered format that binds a secondary virus such as MERS-CoV include: a heavy chain with three CDRs including the amino acid sequences GGTFSSYA (SEQ ID NO: 135), IIPIFGTA (SEQ ID NO: 141), and ARVGYCSSTSCHIGAFDI (SEQ ID NO: 142) respectively and/or a light chain with three CDRs including the amino acid sequences QSVSSS (SEQ ID NO: 143), DSS, and QQYSSSPYT (SEQ ID NO: 144) respectively.

Exemplary binding fragments that can be used in an engineered format that binds a secondary virus such as MERS-CoV include: a heavy chain with three CDRs including the amino acid sequences GGTFSSYA (SEQ ID NO: 135), IIPIFGTA (SEQ ID NO: 141), and ARASYCSTTSCASGAFDI (SEQ ID NO: 147) respectively and/or a light chain with three CDRs including the amino acid sequences QSVLYSSNN NY (SEQ ID NO: 148), WAS, and QQYYSVPFT (SEQ ID NO: 149) respectively.

Exemplary binding fragments that can be used in an engineered format that binds a secondary virus such as MERS-CoV include: a heavy chain with three CDRs including the amino acid sequences GYTFNVYA (SEQ ID NO: 150), IIPILGIA (SEQ ID NO: 151), and ARDYYGSGARGFDY (SEQ ID NO: 152) respectively and/or a light chain with three CDRs including the amino acid sequences SNNVGNQG (SEQ ID NO: 153), TNN, and ASWDSSLSVWV (SEQ ID NO: 154) respectively.

Exemplary binding fragments that can be used in an engineered format that binds a secondary virus such as MERS-CoV include: a heavy chain with three CDRs including the amino acid sequences GGTFSSYA (SEQ ID NO: 135), IIPIFGIA (SEQ ID NO: 156), and ASSNYYGSGSYYPRSAFDI (SEQ ID NO: 157) respectively and/or a light chain with three CDRs including the amino acid sequences QSISND (SEQ ID NO: 158), GAS, and QQYGVSPLT (SEQ ID NO: 159) respectively. Additional examples can be found in U.S. Pat. No. 9,718,872.

Exemplary variable heavy and light chains that can be used in an engineered format that binds a secondary virus such as SARS-CoV include the variable heavy chain sequence including QVQLVQSGAEVKKPGASVKVSCKASGYTFTSYGISWVRQAPGQGLEWMGWISAYNGNTNYA QKLQGRVTMTTDTSTNTAYMELRSLRSDDTAVYYCAVGRYLDYWGQGTLVTV (SEQ ID NO: 160) and the variable light chain sequence including

### DIQMTQSPSSLSASVGDRVTITCRASQDIRNDLGWYQQKPGKAPKRLIYAASSLQSGVPSRFS GSGSGTEFTLTISSLQPEDFATYYCLQYNSYPLTFGGGTKVEIK (SEQ ID NO: 161).

Exemplary variable heavy and light chains that can be used in an engineered format that binds a secondary virus such as SARS-CoV include the variable heavy chain sequence including QVQLVQSGAEVKKPGASVKVSCKASGYTFTSYGISWVRQAPGQGLEWMGWISAYNGNTNYA QKLQGRVTMTTDTSTSTAYMELRSLRSDDTAVYYCAVGRYLDYWGQGTLVTVSS (SEQ ID NO: 162) and the variable light chain sequence including

### DIQMTQSPSSLSASVGDRVTITCRASQDIRNDLGWYQQKPGKAPKRLIYAASSLQSGVPSRFS GSGSGTEFTLTISSLQPEDFATYYCLQYNSYPLTFGGGTKVEIK (SEQ ID NO: 161).

Exemplary variable heavy and light chains that can be used in an engineered format that binds a secondary virus such as SARS-CoV include the variable heavy chain sequence including

QVHLVQSGAEVKKPGASVKVSCKASGYTFTGYYVHWVRQAPGQGLEWMGWINPNSGGTNY AQKFQGRVTMTRDTSISTAYMELSRLRSDDTAVYFCAGGRYLDYWGQGTLVTVSS (SEQ ID NO: 164) and the variable light chain sequence including
DIQMTQSPSSLSASVGDRVTITCRASQGIRNDLGWYQQKSGKAPKRLIYAASSLQSGVPSRFS GSGSGTEFTLTISSLQPEDFATYYCLQYNSYPITFGQGTRLEIK (SEQ ID NO: 165). Additional examples can be found in U.S. Pat. No. 7,728,110.

In particular embodiments, binding fragments disclosed herein can be used to create bi-tri-, (or more) specific immune cell engaging antibody constructs. An example of a multi-specific immune cell engaging antibody construct includes those which bind both a viral epitope on HPIV3, HPIV1, RSV or HMPV and an immune cell (e.g., T-cell or NK-cells) activating epitope, with the goal of bringing immune cells to virally infected cells displaying a viral HPIV3, HPIV1, RSV or HMPV epitope bound by an antibody disclosed herein. See, for example, US 2008/0145362. Such constructs are referred to herein as immune-activating multi-specifics or I-AMS). BiTEs® (Amgen, Thousand Oaks, CA) are one form of I-AMS. Immune cells that can be targeted for localized activation by I-AMS within the current disclosure include, for example, T-cells, natural killer (NK) cells, and macrophages which are discussed in more detail herein.

T-cell activation can be mediated by two distinct signals: those that initiate antigen-dependent primary activation and provide a T-cell receptor like signal (primary cytoplasmic signaling sequences) and those that act in an antigen-independent manner to provide a secondary or co-stimulatory signal (secondary cytoplasmic signaling sequences). I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD 137), OX40, lymphocyte function-associated antigen-1 (LFA-1), LIGHT, NKG2C, and B7-H3.

In particular embodiments, the CD3 binding fragment (e.g., scFv) is derived from the OKT3 antibody (the same as the one utilized in blinatumomab). The OKT3 antibody is described in detail in U.S. Pat. No. 5,929,212. It includes a variable light chain including a CDRL1 sequence including SASSSVSYMN (SEQ ID NO: 67), a CDRL2 sequence including RWIYDTSKLAS (SEQ ID NO: 68), and a CDRL3 sequence including QQWSSNPFT (SEQ ID NO: 69). In particular embodiments, the CD3 T-cell activating epitope binding fragment is a human or humanized binding fragment (e.g., scFv) including a variable heavy chain including a CDRH1 sequence including KASGYTFTRYTMH (SEQ ID NO: 70), a CDRH2 sequence including INPSRGYTNYNQKFKD (SEQ ID NO: 71), and a CDRH3 sequence including YYDDHYCLDY (SEQ ID NO: 72).

The following sequence is an scFv derived from OKT3 which retains the capacity to bind CD3:

(SEQ ID NO: 73)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSSGGGGSGGGGSGGGGSQIVLTQSPAIMS
ASPGEKVTMTCSASSSVSYMNWYQQKSGTSPKRWIYDTSKLASGVPAHF
RGSGSGTSYSLTISGMEAEDAATYYCQQWSSNPFTFGSGTKLEINR.

It may also be used as a CD3 binding fragment.

In particular embodiments, the CD3 T-cell activating epitope binding fragment is a human or humanized binding fragment (e.g., scFv) including a variable light chain including a CDRL1 sequence including QSLVHNNGNTY (SEQ ID NO: 74), a CDRL2 sequence including KVS, and a CDRL3 sequence including GQGTQYPFT (SEQ ID NO: 75). In particular embodiments, the CD3 T-cell activating epitope binding fragment is a human or humanized binding fragment (e.g., scFv) including a variable heavy chain including a CDRH1 sequence including GFTFTKAW (SEQ ID NO: 76), a CDRH2 sequence including IKDKSNSYAT (SEQ ID NO: 77), and a CDRH3 sequence including RGVYYALSPFDY (SEQ ID NO: 78). These reflect CDR sequences of the 20G6-F3 antibody.

In particular embodiments, the CD3 T-cell activating epitope binding fragment is a human or humanized binding fragment (e.g., scFv) including a variable light chain including a CDRL1 sequence including QSLVHDNGNTY (SEQ ID NO: 79), a CDRL2 sequence including KVS, and a CDRL3 sequence including GQGTQYPFT (SEQ ID NO: 75). In particular embodiments, the CD3 T-cell activating epitope binding fragment is a human or humanized binding fragment (e.g., scFv) including a variable heavy chain including a CDRH1 sequence including GFTFSNAW (SEQ ID NO: 80), a CDRH2 sequence including IKARSNNYAT (SEQ ID NO: 81), and a CDRH3 sequence including RGTYYASKPFDY (SEQ ID NO: 82). These reflect CDR sequences of the 4B4-D7 antibody.

In particular embodiments, the CD3 T-cell activating epitope binding fragment is a human or humanized binding fragment (e.g., scFv) including a variable light chain including a CDRL1 sequence including QSLEHNNGNTY (SEQ ID NO: 83), a CDRL2 sequence including KVS; not included in Sequence Listing), and a CDRL3 sequence including GQGTQYPFT (SEQ ID NO: 75).

In particular embodiments, the CD3 T-cell activating epitope binding fragment is a human or humanized binding fragment (e.g., scFv) including a variable heavy chain including a CDRH1 sequence including GFTFSNAW (SEQ ID NO: 80), a CDRH2 sequence including IKDKSNNYAT (SEQ ID NO: 84), and a CDRH3 sequence including RYVHYGIGYAMDA (SEQ ID NO: 85). These reflect CDR sequences of the 4E7-C9 antibody.

In particular embodiments, the CD3 T-cell activating epitope binding fragment is a human or humanized binding fragment (e.g., scFv) including a variable light chain including a CDRL1 sequence including QSLVHTNGNTY (SEQ ID NO: 86), a CDRL2 sequence including KVS, and a CDRL3 sequence including GQGTHYPFT (SEQ ID NO: 87). In particular embodiments, the CD3 T-cell activating epitope binding fragment is a human or humanized binding fragment (e.g., scFv) including a variable heavy chain including a CDRH1 sequence including GFTFTNAW (SEQ ID NO: 88), a CDRH2 sequence including KDKSNNYAT (SEQ ID NO: 89), and a CDRH3 sequence including RYVHYRFAYALDA (SEQ ID NO: 90). These reflect CDR sequences of the 18F5-H10 antibody.

Additional examples of anti-CD3 antibodies, binding fragments, and CDRs can be found in WO2016/116626. TR66 may also be used.

CD28 is a surface glycoprotein present on 80% of peripheral T-cells in humans and is present on both resting and activated T-cells. CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding fragment (e.g., scFv) is derived from CD80, CD86 or the 9D7 antibody. Additional antibodies that bind CD28 include 9.3, KOLT-2, 15E8, 248.23.2, and EX5.3D10. Further, 1YJD provides a crystal structure of human CD28 in complex with the Fab fragment of a mitogenic antibody (5.11A1).

In particular embodiments, a CD28 binding fragment is derived from TGN1412. In particular embodiments, the variable heavy chain of TGN1412 includes:

(SEQ ID NO: 91)
QVQLVQSGAEVKKPGASVKVSCKASGYTFTSYYIHWVRQAPGQGLEWIG
CIYPGNVNTNYNEKFKDRATLTVDTSISTAYMELSRLRSDDTAVYFCTR
SHYGLDWNFDVWGQGTTVTVSS

and the variable light chain of TGN1412 includes:

(SEQ ID NO: 92)
DIQMTQSPSSLSASVGDRVTITCHASQNIYVWLNWYQQKPGKAPKLLIY
KASNLHTGVPSRFSGSGSGTDFTLTISSLQPEDFATYYCQQGQTYPYTF
GGGTKVEIK.

In particular embodiments, the CD28 binding fragment includes a variable light chain including a CDRL1 sequence including HASQNIYVWLN (SEQ ID NO: 93), CDRL2 sequence including KASNLHT (SEQ ID NO: 94), and CDRL3 sequence including QQGQTYPYT (SEQ ID NO: 95), a variable heavy chain including a CDRH1 sequence including GYTFTSYYIH (SEQ ID NO: 96), a CDRH2 sequence including CIYPGNVNTNYNEK (SEQ ID NO: 97), and a CDRH3 sequence including SHYGLDWNFDV (SEQ ID NO: 98).

In particular embodiments, the CD28 binding fragment including a variable light chain including a CDRL1 sequence including HASQNIYVWLN (SEQ ID NO: 93), a CDRL2 sequence including KASNLHT (SEQ ID NO: 94), and a CDRL3 sequence including QQGQTYPYT (SEQ ID NO: 95) and a variable heavy chain including a CDRH1 sequence including SYYIH (SEQ ID NO: 99), a CDRH2 sequence including CIYPGNVNTNYNEKFKD (SEQ ID NO: 100), and a CDRH3 sequence including SHYGLDWNFDV (SEQ ID NO: 98).

Activated T-cells express 4-1BB (CD137). In particular embodiments, the 4-1BB binding fragment includes a variable light chain including a CDRL1 sequence including RASQSVS (SEQ ID NO: 101), a CDRL2 sequence including ASNRAT (SEQ ID NO: 102), and a CDRL3 sequence including QRSNWPPALT (SEQ ID NO: 103) and a variable heavy chain including a CDRH1 sequence including YYWS (SEQ ID NO: 104), a CDRH2 sequence including INH, and a CDRH3 sequence including YGPGNYDWYFDL (SEQ ID NO: 105).

In particular embodiments, the 4-1EE binding fragment includes a variable light chain including a CDRL1 sequence including SGDNIGDQYAH (SEQ ID NO: 106), a CDRL2 sequence including QDKNRPS (SEQ ID NO: 107), and a CDRL3 sequence including ATYTGFGSLAV (SEQ ID NO: 108) and a variable heavy chain including a CDRH1 sequence including GYSFSTYWIS (SEQ ID NO: 109), a CDRH2 sequence including KIYPGDSYTNYSPS (SEQ ID NO: 110) and a CDRH3 sequence including GYGIFDY (SEQ ID NO: 111).

Particular embodiments disclosed herein including binding fragments that bind epitopes on CD8. In particular embodiments, the CD8 binding fragment (e.g., scFv) is derived from the OKT8 antibody. For example, in particular embodiments, the CD8 T-cell activating epitope binding fragment is a human or humanized binding fragment (e.g., scFv) including a variable light chain including a CDRL1 sequence including RTSRSISQYLA (SEQ ID NO: 112), a CDRL2 sequence including SGSTLQS (SEQ ID NO: 113), and a CDRL3 sequence including QQHNENPLT (SEQ ID NO: 114). In particular embodiments, the CD8 T-cell activating epitope binding fragment is a human or humanized binding fragment (e.g., scFv) including a variable heavy chain including a CDRH1 sequence including GFNIKD (SEQ ID NO: 115), a CDRH2 sequence including RIDPANDNT (SEQ ID NO: 116), and a CDRH3 sequence including GYGYYVFDH (SEQ ID NO: 117). These reflect CDR sequences of the OKT8 antibody.

In particular embodiments natural killer cells (also known as NK-cells, K-cells, and killer cells) are targeted for localized activation by I-AMS. NK cells can induce apoptosis or cell lysis by releasing granules that disrupt cellular membranes and can secrete cytokines to recruit other immune cells.

Examples of activating proteins expressed on the surface of NK cells include NKG2D, CD8, CD16, KIR2DL4, KIR2DS1, KIR2DS2, KIR3DS1, NKG2C, NKG2E, NKG2D, and several members of the natural cytotoxicity receptor (NCR) family. Examples of NCRs that activate NK cells upon ligand binding include NKp30, NKp44, NKp46, NKp80, and DNAM-1.

Examples of commercially available antibodies that bind to an NK cell receptor and induce and/or enhance activation of NK cells include: 5C6 and 1D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16.

In particular embodiments, the I-AMS can bind to and block an NK cell inhibitory receptor to enhance NK cell activation. Examples of NK cell inhibitory receptors that can be bound and blocked include KIR2DL1, KIR2DL2/3, KIR3DL1, NKG2A, and KLRG1. In particular embodiments, a binding fragment that binds and blocks the NK cell inhibitory receptors KIR2DL1 and KIR2DL2/3 includes a variable light chain region of the sequence

EIVLTQSPVTLSLSPGERATLSCRASQSVSSYLAWYQQKPGQAPRLLIYDASNRATGIPARFSG SGSGTDFTLTISSLEPEDFAVYYCQQRSNWMYTFGQGTKLEIKRT (SEQ ID NO: 118) and a variable heavy chain region of the sequence
QVQLVQSGAEVKKPGSSVKVSCKASGGTFSFYAISWVRQAPGQGLEWMGGFIPIFGAANYAQ KFQGRVTITADESTSTAYMELSSLRSDDTAVYYCARIPSGSYYYDYDMDVWGQGTTVTVSS (SEQ ID NO: 119). Additional NK cell activating antibodies are described in WO/2005/0003172 and U.S. Pat. No. 9,415,104.

In particular embodiments macrophages are targeted for localized activation by I-AMS. Macrophages are a type of leukocyte (or white blood cell) that can engulf and digest cells, cellular debris, and/or foreign substances in a process known as phagocytosis.

The I-AMS can be designed to bind to a protein expressed on the surface of macrophages. Examples of activating proteins expressed on the surface of macrophages (and their precursors, monocytes) include CD11b, CD11c, CD64, CD68, CD119, CD163, CD206, CD209, F4/80, IFGR2 Toll-like receptors (TLRs) 1-9, IL-4Ra, and MARCO. Commercially available antibodies that bind to proteins expressed on the surface of macrophages include M1/70, which binds and activates CD11b (available from BioLegend®); KP1, which binds and activates CD68 (available from ABCAM®, Cambridge, United Kingdom); and ab87099, which binds and activates CD163 (available from ABCAM®).

In particular embodiments, I-AMS can target a pathogen recognition receptor (PRR). PRRs are proteins or protein complexes that recognize a danger signal and activate and/or enhance the innate immune response. Examples of PRRs include the TLR4/MD-2 complex, which recognizes gram negative bacteria; Dectin-1 and Dectin-2, which recognize mannose moieties on fungus and other pathogens; TLR2/TLR6 or TLR2/TLR1 heterodimers, which recognize gram positive bacteria; TLR5, which recognizes flagellin; and TLR9 (CD289), which recognizes CpG motifs in DNA. In particular embodiments, I-AMS can bind and activate TLR4/MD-2, Dectin-1, Dectin-2, TRL2/TLR6, TLR2/TLR1, TLR5, and/or TLR9.

In particular embodiments, I-AMS can target the complement system. The complement system refers to an immune pathway that is induced by antigen-bound antibodies and involves signaling of complement proteins, resulting in immune recognition and clearance of the antibody-coated antigens.

Binding fragments of I-AMS and other engineered formats described herein may be joined through a linker. A linker is an amino acid sequence which can provide flexibility and room for conformational movement between the binding fragments of a I-AM. Any appropriate linker may be used.

Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ (SEQ ID NO: 120), wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 121), (Gly₃Ser)ₙ(Gly₄Ser)ₙ (SEQ ID NO: 122), (Gly₃Ser)ₙ(Gly₂Ser)ₙ (SEQ ID NO: 123), and (Gly₃Ser)ₙ(Gly₄Ser)₁ (SEQ ID NO: 124). In particular embodiments, the linker is (Gly₄Ser)₄ (SEQ ID NO: 125), (Gly₄Ser)₃ (SEQ ID NO: 126), (Gly₄Ser)₂ (SEQ ID NO: 127), (Gly₄Ser)₁ (SEQ ID NO: 128), (Gly₃Ser)₂ (SEQ ID NO: 129), (Gly₃Ser)₁ (SEQ ID NO: 130), (Gly₂Ser)₂ (SEQ ID NO: 131) or (Gly₂Ser)₁, GGSGGGSGGSG (SEQ ID NO: 132), GGSGGGSGSG (SEQ ID NO: 133), or GGSGGGSG (SEQ ID NO: 134).

Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding fragments needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Cytolytic properties of I-AMS molecules can be confirmed in comparative in vitro assays. Briefly, for cell line experiments, target virally-infected cells can be incubated in 96-well round bottom plates at 5-10,000 cells/well containing increasing concentrations of the various I-AMS antibodies (e.g., viral-epitope/CD3-AMS including a viral epitope-CD3 bispecific antibody (BiAb)) with/without healthy donor T-cells (used at an E:T cell ratio of 1:1 and 3:1). After 48 hours, cell numbers and drug-induced cytotoxicity, using 4′,6-diamidino-2-phenylindole (DAPI) to detect non-viable cells, can be determined by flow cytometry. In experiments where healthy donor T-cells are added, virally-infected cells can be identified by forward/side scatter properties and negativity for CellVue Burgundy dye. Experiments can include technical duplicates.

In particular embodiments including I-AMS constructs, T-cell activating epitope binding fragments include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{α}, V_{β}, C_{α}, or C_{β} of a known TCR. An insertion, deletion or substitution may be anywhere in a V_{α}, V_{β}, C_{α}, or C_{β} region, including at the amino- or carboxy-terminus or both ends of these regions, provided that each CDR includes zero changes or at most one, two, or three changes and provided a binding fragment including a modified V_{α}, V_{β}, C_{α}, or C_{β} region can still specifically bind its target with an affinity similar to wild type.

In particular embodiments, bispecific molecules can be assembled by synthesizing each scFv as a DNA fragment with overlapping Gibson assembly-compatible ends in the canonical BiTE® antibody format. Prototypical intervening regions such as (Gly₄Ser)₃ (SEQ ID NO: 126) linkers can be used between paired variable domains and a short Gly₄Ser (SEQ ID NO: 128) linker between the two scFvs.

Anti-viral tri-specific antibodies are artificial proteins that simultaneously bind to three different types of antigens, wherein at least one of the antigens is a viral epitope on HPIV1, HPIV3, RSV or HMPV bound by an antibody disclosed herein. Tri-specific antibodies are described in, for example, WO2016/105450, WO 2010/028796; WO 2009/007124; WO 2002/083738; US 2002/0051780; and WO 2000/018806.

(ii) Formulations. Any of the antibodies described herein in any exemplary format can be formulated alone or in combination into compositions for administration to subjects. Salts and/or pro-drugs of the antibodies can also be used.

A pharmaceutically acceptable salt includes any salt that retains the activity of the antibody and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

In particular embodiments, the compositions include antibodies of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. Hank's solution refers to an isotonic buffer solution including inorganic salts and a carbohydrate. Ringer's solution includes sodium chloride, potassium chloride, calcium chloride at physiologic concentrations with sodium bicarbonate (or sodium lactate) to balance pH. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

(iii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of an infection's development, progression, and/or resolution.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection. In particular embodiments, prophylactic treatments reduce, delay, or prevent the worsening of an infection.

A “therapeutic treatment” includes a treatment administered to a subject who displays symptoms or signs of an infection and is administered to the subject for the purpose of diminishing or eliminating those signs or symptoms of the infection. The therapeutic treatment can reduce, control, or eliminate the presence or activity of the infection and/or reduce control or eliminate side effects of the infection.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a reducing or preventing a virus from infecting a cell, decreasing the number of infected cells, decreasing the volume of infected tissue, increasing lifespan, increasing life expectancy, reducing or eliminating infection-associated symptoms (e.g., reduced lung capacity). In particular embodiments, therapeutically effective amounts induce an immune response. The immune response can be against a virus.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of infection, stage of infection, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

As indicated, particular methods include uses in susceptible individuals, such as those undergoing HCT, lung transplant recipients, premature infants, adults over age 65, or those with other health-related issues that increase susceptibility to infection with respiratory viruses.

The pharmaceutical compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (iv) Exemplary Embodiments

1. An antibody or binding fragment thereof including the complementarity determining regions (CDRs) of PI3-E12, PI3-A3, PI3-B5, PI3-A10, PI3-A12, 3×1, MxR-B11, or MxR-D10.
2. An antibody or binding fragment of embodiment 1, wherein the CDRs are according to Kabat, Chothia, Martin, Contact, IMGT, AHo, or North numbering.
3. An antibody or binding fragment of embodiment 1, having
a CDRH1 including GFTFSDHY (SEQ ID NO: 1); a CDRH2 including ISSSGSNT (SEQ ID NO: 2); a CDRH3 including ARAKWGTMGRGAPPTIYDH (SEQ ID NO: 3); a CDRL1 including QSLLQSNGNNY (SEQ ID NO: 4); a CDRL2 including LGS; and a CDRL3 including MQALQTPLT (SEQ ID NO: 5);
a CDRH1 including GFTFSNYW (SEQ ID NO: 8); a CDRH2 including VKEEGSEK (SEQ ID NO: 9); a CDRH3 including AGEVKSGWFGRYFDS (SEQ ID NO: 10); a CDRL1 including QSVGSW (SEQ ID NO: 11); a CDRL2 including KTS; and a CDRL3 including QQYSSFPYT (SEQ ID NO: 12);
a CDRH1 including GYNFTNYW (SEQ ID NO: 15); a CDRH2 including IYPADSDT (SEQ ID NO: 16); a CDRH3 including ARPSTRWFVPGGMDV (SEQ ID NO: 17); a CDRL1 including QSIGAW (SEQ ID NO: 18); a CDRL2 including KAS; and a CDRL3 including QQHSSYPST (SEQ ID NO: 19);
a CDRH1 including GFNFNNYG (SEQ ID NO: 22); a CDRH2 including VSFDGSNR (SEQ ID NO: 23); a CDRH3 including SKSKYSDFWSEI (SEQ ID NO: 24); a CDRL1 including QNVMRY (SEQ ID NO: 25); a CDRL2 including DAS; and a CDRL3 including QQRTNHRFS (SEQ ID NO: 26);
a CDRH1 including GDSVKSDDFY (SEQ ID NO: 29); a CDRH2 including IYYGGRT (SEQ ID NO: 30); a CDRH3 including VRVEGLLWFGELFDY (SEQ ID NO: 31); a CDRL1 including NSNIGNNF (SEQ ID NO: 32); a CDRL2 including KDY; and a CDRL3 including AAWQDGLSGPL (SEQ ID NO: 33);
a CDRH1 including GFTFSSFG (SEQ ID NO: 36); a CDRH2 including ISHSAGFL (SEQ ID NO: 37); a CDRH3 including AKRLAGLPDLEWLLYPNFLDH (SEQ ID NO: 38); a CDRL1 including ILRTYY (SEQ ID NO: 39); a CDRL2 including GKN; and a CDRL3 including SSRDRSGNHVL (SEQ ID NO: 40);
a CDRH1 including GFPFSSYK (SEQ ID NO: 43); a CDRH2 including ISASGSYI (SEQ ID NO: 44); a CDRH3 including ARDGGRELSPFEK (SEQ ID NO: 45); a CDRL1 including NSNIGTGYD (SEQ ID NO: 46); a CDRL2 including DNN; and a CDRL3 including QSYDKSLGGWV (SEQ ID NO: 47); or
a CDRH1 including GFIFSNYD (SEQ ID NO: 50); a CDRH2 including ITGGSSFI (SEQ ID NO: 51); a CDRH3 including ARDGGRQLSPCEH (SEQ ID NO: 52); a CDRL1 including SSNIGAGYD (SEQ ID NO: 53); a CDRL2 including DNN; and a CDRL3 including QSYDRGLSGWA (SEQ ID NO: 54).
   4. An antibody or binding fragment thereof including
a variable heavy chain having a sequence with at least 90% or at least 95% sequence identity to the sequence as set forth in SEQ ID NO: 166 and a variable light chain having a sequence with at least 90% or at least 95% sequence identity to the sequence as set forth in SEQ ID NO: 167;
a variable heavy chain having a sequence with at least 90% or at least 95% sequence identity to the sequence as set forth in SEQ ID NO: 168 and a variable light chain having a sequence with at least 90% or at least 95% sequence identity to the sequence as set forth in SEQ ID NO: 169;
a variable heavy chain having a sequence with at least 90% or at least 95% sequence identity to the sequence as set forth in SEQ ID NO: 170 and a variable light chain having a sequence with at least 90% or at least 95% sequence identity to the sequence as set forth in SEQ ID NO: 171;
a variable heavy chain having a sequence with at least 90% or at least 95% sequence identity to the sequence as set forth in SEQ ID NO: 172 and a variable light chain having a sequence with at least 90% or at least 95% sequence identity to the sequence as set forth in SEQ ID NO: 173;
a variable heavy chain having a sequence with at least 90% or at least 95% sequence identity to the sequence as set forth in SEQ ID NO: 174 and a variable light chain having a sequence with at least 90% or at least 95% sequence identity to the sequence as set forth in SEQ ID NO: 175;
a variable heavy chain having a sequence with at least 90% or at least 95% sequence identity to the sequence as set forth in SEQ ID NO: 176 and a variable light chain having a sequence with at least 90% or at least 95% sequence identity to the sequence as set forth in SEQ ID NO: 177;
a variable heavy chain having a sequence with at least 90% or at least 95% sequence identity to the sequence as set forth in SEQ ID NO: 178 and a variable light chain having a sequence with at least 90% or at least 95% sequence identity to the sequence as set forth in SEQ ID NO: 179; or
a variable heavy chain having a sequence with at least 90% or at least 95% sequence identity to the sequence as set forth in SEQ ID NO: 180 and a variable light chain having a sequence with at least 90% or at least 95% sequence identity to the sequence as set forth in SEQ ID NO: 181.
   5. An antibody or binding fragment thereof having
a variable heavy chain including the sequence as set forth in SEQ ID NO: 166 and a variable light chain including the sequence as set forth in SEQ ID NO: 167;
a variable heavy chain including the sequence as set forth in SEQ ID NO: 168 and a variable light chain including the sequence as set forth in SEQ ID NO: 169;
a variable heavy chain including the sequence as set forth in SEQ ID NO: 170 and a variable light chain including the sequence as set forth in SEQ ID NO: 171;
a variable heavy chain including the sequence as set forth in SEQ ID NO: 172 and a variable light chain including the sequence as set forth in SEQ ID NO: 173;
a variable heavy chain including the sequence as set forth in SEQ ID NO: 174 and a variable light chain including the sequence as set forth in SEQ ID NO: 175;
a variable heavy chain including the sequence as set forth in SEQ ID NO: 176 and a variable light chain including the sequence as set forth in SEQ ID NO: 177;
a variable heavy chain including the sequence as set forth in SEQ ID NO: 178 and a variable light chain including the sequence as set forth in SEQ ID NO: 179; or
a variable heavy chain including the sequence as set forth in SEQ ID NO: 180 and a variable light chain including the sequence as set forth in SEQ ID NO: 181
   6. An antibody or binding fragment thereof encoded by a sequence having
at least 90% or at least 95% sequence identity to the sequence as set forth in SEQ ID NO: 6 and SEQ ID NO: 7;
at least 90% or at least 95% sequence identity to the sequence as set forth in SEQ ID NO: 13 and SEQ ID NO: 14;
at least 90% or at least 95% sequence identity to the sequence as set forth in SEQ ID NO: 27 and SEQ ID NO: 28;
at least 90% or at least 95% sequence identity to the sequence as set forth in SEQ ID NO: 34 and SEQ ID NO: 35;
at least 90% or at least 95% sequence identity to the sequence as set forth in SEQ ID NO: 41 and SEQ ID NO: 42;
at least 90% or at least 95% sequence identity to the sequence as set forth in SEQ ID NO: 48 and SEQ ID NO: 49; or
at least 90% or at least 95% sequence identity to the sequence as set forth in SEQ ID NO: 55 and SEQ ID NO: 56.
   7. An antibody or binding fragment thereof encoded by a sequence including the sequence as set forth in SEQ ID NO: 6 and SEQ ID NO: 7; SEQ ID NO: 13 and SEQ ID NO: 14; SEQ ID NO: 27 and SEQ ID NO: 28; SEQ ID NO: 34 and SEQ ID NO: 35; SEQ ID NO: 41 and SEQ ID NO: 42; SEQ ID NO: 48 and SEQ ID NO: 49; or SEQ ID NO: 55 and SEQ ID NO: 56.
   8. The antibody or binding fragment thereof of any of embodiments 1-7, including an IgG with M252Y, S254T, and T256E mutations in the Fc region.
   9. A binding molecule including a binding fragment of at least 2 antibodies of any of embodiments 1-8.
   10. The binding molecule of embodiment 9, wherein the binding fragments of at least 2 antibodies of any of embodiments 1-3 include Fab or single chain variable fragments (scFv).
   11. The binding molecule of embodiment 9 or 10, wherein the binding fragments include Fab linked through a knobs-into-holes linkage of heavy chains (FIG. 8, top).
   12. The binding molecule of any of embodiments 9-11, wherein the binding fragments include an ScFv with one binding fragment linked to the constant region of an antibody having a different binding fragment (FIG. 8, bottom).
   13. The binding molecule of embodiment 12, wherein the linker includes a Gly-Ser linker.
   14. The binding molecule of any of embodiments 9-13, wherein the binding fragments of at least 2 antibodies of any of embodiments 1-8 include scFv.
   15. The binding molecule of any of embodiments 9-14, including
a binding fragment of a PI3 antibody and a binding fragment of an MxR antibody;
a binding fragment of a PI3 antibody and a binding fragment of the 3×1 antibody; or
a binding fragment of the 3×1 antibody and a binding fragment of an MxR antibody.
   16. The binding molecule of any of embodiments 9-15, including a binding fragment of a PI3 antibody, a binding fragment of the 3×1 antibody, and a binding fragment of an MxR antibody.
   17. The binding molecule of embodiment 15, wherein the binding fragments include Fab and/or scFv.
   18. The binding molecule of any of embodiments 9-17, including the MxR-B11 Fab and the 3×1 Fab; a MxR-B11 scFv and the 3×1 Fab; the MxR-B11 Fab and a 3×1 scFv; or a MxR-B11 scFv and a 3×1 scFv.
   19. The binding molecule of any of embodiments 9-18, including the MxR-D10 Fab and the 3×1 Fab; a MxR-D10 scFv and the 3×1 Fab; the MxR-D10 Fab and a 3×1 scFv; or a MxR-D10 scFv and a 3×1 scFv.
   20. A binding molecule including a binding fragment of any of embodiments 1-8 and a binding fragment of an antibody that binds a secondary virus.
   21. The binding molecule of embodiment 20, wherein the secondary virus is selected from an adenovirus, a boca virus, a coronavirus, an enterovirus, an influenza virus, a metapneumovirus, a parainfluenza virus, and/or a rhinovirus.
   22. The binding molecule of embodiment 20 or 21, wherein the secondary virus is selected from human adenovirus, human boca virus (HBoV), and/or human coronavirus (HCoV).
   23. The binding molecule of any of embodiments 20-22, wherein the secondary virus is a coronavirus selected from SARS-CoV, MERS-CoV, coronavirus 229E, coronavirus OC43, coronavirus NL63, coronavirus HKU1, coronavirus NL, and/or coronavirus NH.
   24. The binding molecule of any of embodiments 20-23, wherein the secondary virus is selected from influenza group A, influenza group B, human parainfluenza virus 2 (HPIV2), HPIV4, human rhinovirus (HRV)A, HRVB, and/or HRVC.
   25. The binding molecule of any of embodiments 20-34, wherein the binding fragment includes the CDRs of antibody 8C4, 5Hx-1, 5Hx-2, 5Hx-3, 5Hx-4, 5Hx-5, 5.100K-1, 5PB-1, 5Fb-1, 1E11, EPR23305-44 47D11, CR3022, CDC2-A2, G2, 5F9, FIB-H1, JC57-13, 32D6, CH65, CR9114, MAb 22/1, MAb70/1, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, PEG-1, CR8033, CR8071, 113/2, 124/4, 128/2, 134/1, 146/1, 152/2, 160/1, 162/1, 195/3, 206/2, 238/4, 280/2, PAR2 (boca231/9F), or TCN-711.
   26. A binding molecule including a binding fragment of any of embodiments 1-8 and a binding fragment that activates an immune cell.
   27. The binding molecule of embodiment 26, wherein the binding fragment that activates an immune cell binds CD3, CD28, or 4-1BB.
   28. The binding molecule of embodiment 26 or 27, wherein the binding fragment that activates an immune cell includes the CDRs of OKT3 or TGN1412.
   29. A composition including an antibody or binding fragment of any of embodiments 1-28 and a pharmaceutically acceptable carrier.
   30. The composition of embodiment 29, wherein the pharmaceutically-acceptable carrier includes an aqueous solution.
   31. The composition of embodiment 29 or 30, wherein the pharmaceutically-acceptable carrier includes physiological saline.
   32. The composition of any of embodiments 29-31, wherein the pharmaceutically-acceptable carrier includes sodium chloride, potassium chloride and calcium chloride.
   33. The composition of any of embodiments 29-32, wherein the pharmaceutically-acceptable carrier includes sodium bicarbonate or sodium lactate.
   34. The composition of any of embodiments 29-33, wherein the pharmaceutically-acceptable carrier includes inorganic salts.
   35. The composition of any of embodiments 29-34, wherein the pharmaceutically-acceptable carrier includes a carbohydrate.
   36. The composition of any of embodiments 29-35, wherein the pharmaceutically-acceptable carrier includes an antioxidant, a buffering agent, a chelating agent, an isotonic agent, a preservative, and/or a stabilizer.
   37. The composition of any of embodiments 29-36, wherein the pharmaceutically-acceptable carrier includes a release modifier.
   38. The composition of any of embodiments 29-37, wherein the composition includes a therapeutically-effective amount of the antibody or binding fragment for administration to a subject.
   39. A method of providing an anti-viral effect in a subject in need thereof including administering a therapeutically effective amount of the composition of any of embodiments 29-38 to the subject thereby providing the anti-viral effect.
   40. The method of embodiment 39, wherein the anti-viral effect includes an anti-HPIV3 effect, an anti-HPIV1 effect, an anti-respiratory syncytial virus (RSV) effect, and/or an anti-human metapneumovirus (HMPV) effect.
   41. The method of embodiment 39 or 40, wherein the anti-viral effect includes an anti-HPIV3 effect and an anti-HPIV1 effect.
   42. The method of any of embodiments 39-32, wherein the anti-viral effect includes an anti-RSV effect and an anti-HMPV effect.
   43. The method of any of embodiments 39-42, wherein the anti-viral effect includes an anti-HPIV effect, an anti-RSV effect and an anti-HMPV effect.
   44. The method of any of embodiments 39-43, wherein the subject in need thereof is a hematopoietic stem cell transplant (HCT) recipient, a lung transplant recipient, a premature infant, a person over 65 years of age, a homeless person, or a person with lung disease.
   45. The method of any of embodiments 39-44, wherein the subject has chronic obstructive pulmonary disease (COPD).
   46. The method of any of embodiments 39-45, wherein the anti-viral effect includes an anti-viral effect against a secondary virus.
   47. The method of embodiment 46, wherein the secondary virus is selected from an adenovirus, a boca virus, a coronavirus, an enterovirus, an influenza virus, a metapneumovirus, a parainfluenza virus, and/or a rhinovirus.
   48. The method of embodiment 46 or 47, wherein the secondary virus is selected from human adenovirus, human boca virus (HBoV), and/or human coronavirus (HCoV).
   49. The method of any of embodiments 46-48, wherein the secondary virus is a coronavirus selected from SARS-CoV, MERS-CoV, coronavirus 229E, coronavirus OC43, coronavirus NL63, coronavirus HKU1, coronavirus NL, and/or coronavirus NH.
   50. The method of any of embodiments 46-49, wherein the secondary virus is selected from influenza group A, influenza group B, human parainfluenza virus 2 (HPIV2), HPIV4, human rhinovirus (HRV)A, HRVB, and/or HRVC.

(v) Experimental Examples. Introduction. HPIV3 is a common cause of respiratory illness in infants and children. Over 11,000 hospitalizations per year in the US occur for fever or acute respiratory illness due to HPIV3. HPIV3, like respiratory syncytial virus (RSV), infects early in life and frequently causes severe bronchiolitis and pneumonia in infants under six months of age who are unable to mount a robust antibody response. HPIV3 is also an important cause of mortality, morbidity, and health care costs in other vulnerable populations, particularly immunocompromised hematopoietic stem cell transplant (HCT) recipients. Up to a third of HCT recipients acquire a respiratory viral infection within six months of transplant. In up to a third of those patients, the virus progresses from the upper to the lower respiratory tract. Once the virus gains a foothold in the lower tract, little can be done for most viruses beyond supportive care and up to 40% of patients with lower tract disease die within three months. HPIV3 is an important cause of serious respiratory viral infections after HCT with an incidence of 18% post-transplant at some centers. In the absence of any vaccine or therapy, there is significant need for preventive and therapeutic interventions against HPIV3.

Neutralizing monoclonal antibodies have been correlated with protection against several respiratory viruses, including RSV and influenza. The monoclonal antibody palivizumab is a humanized antibody targeting the Fusion (F) protein of RSV and was licensed for use as immunoprophylaxis to prevent severe disease in high-risk infants. The F protein of RSV is an essential surface glycoprotein and therefore a major neutralizing antibody target. As a class I fusion protein, F mediates viral entry by transitioning between a metastable prefusion (preF) conformation and a stable postfusion (postF) conformation. Since preF is the major conformation on infectious virus, antibodies to preF are potent at neutralizing virus, whereas antibodies targeting postF generally are not (Ngwuta, et al. Sci Transl Med 7, 309ra162, (2015); Gilman, et al. Sci Immunol 1, (2016)). Similar to RSV, the F protein of HPIV3 also adopts a preF and a postF conformation. HPIV3 F was recently stabilized in the prefusion conformation, with the preF-stabilized immunogens eliciting higher neutralizing antibody titers than the same F immunogen in the postF conformation (Stewart-Jones, et al. Proc Natl Acad Sci USA 115, 12265-12270, (2018)). In an effort to isolate monoclonal antibody candidates for prevention and therapy, a high-throughput screening strategy was developed that enabled the rapid selection and testing of human HPIV3 preF-specific B cells for the ability to neutralize HPIV3. This method was applied to isolate several potent neutralizing monoclonal antibodies, characterized their binding, and tested one of the antibody candidates in an in vivo challenge model.

Results. Identification and isotype of HPIV3-specific B cells within the human B cell repertoire. The HPIV3 F protein was biotinylated in either the prefusion (preF) and postfusion (postF) conformation and mixed each with fluorochrome-labeled streptavidin. HPIV3 preF- and postF-specific B cells were then enriched for using magnetic microbeads conjugated to antibodies targeting the fluorochrome. Vaccination of animals with the preF conformation of HPIV3 F was previously found to elicit much higher neutralizing titers than postF (Stewart-Jones, et al. Proc Natl Acad Sci USA 115, 12265-12270, (2018)).

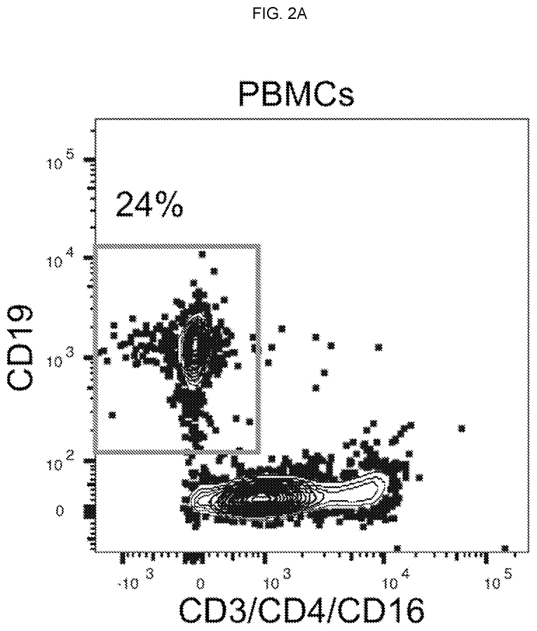

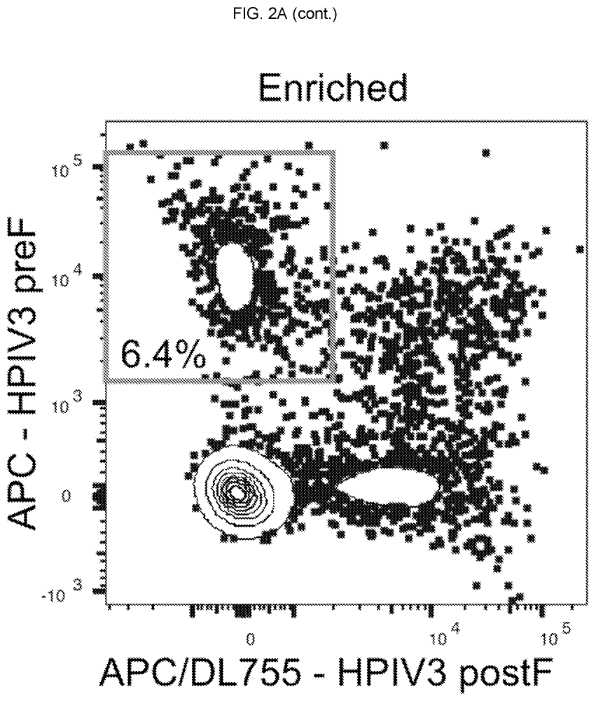

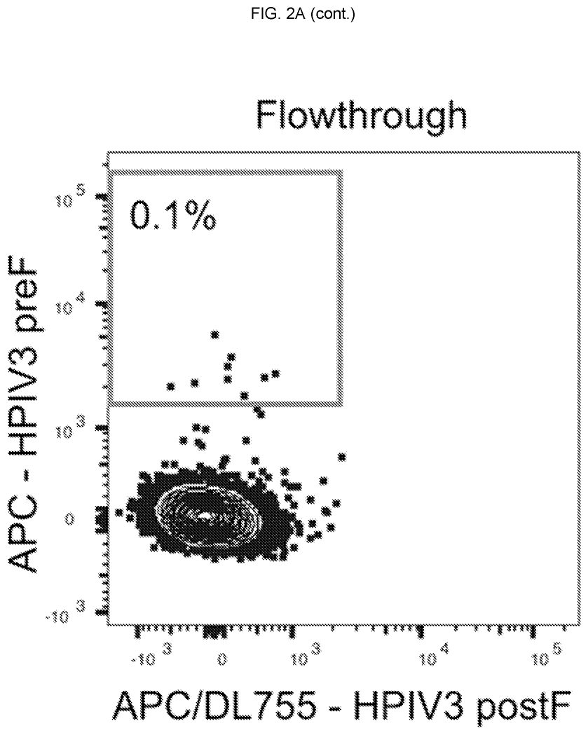

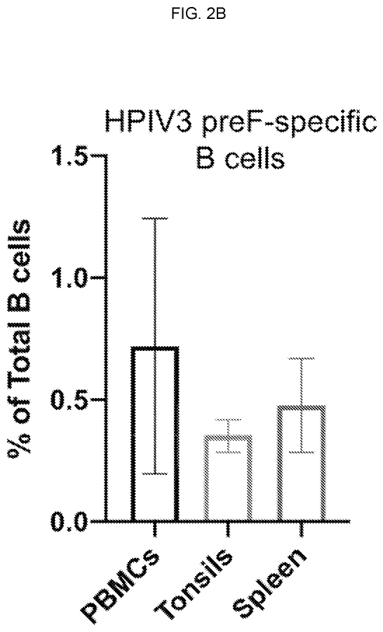

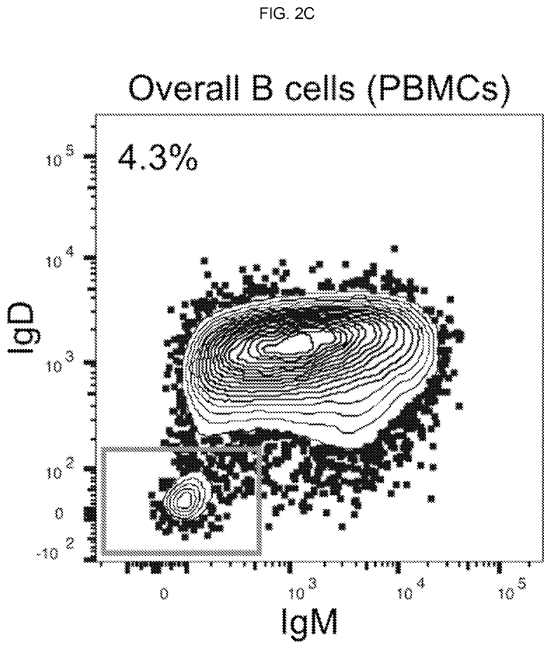

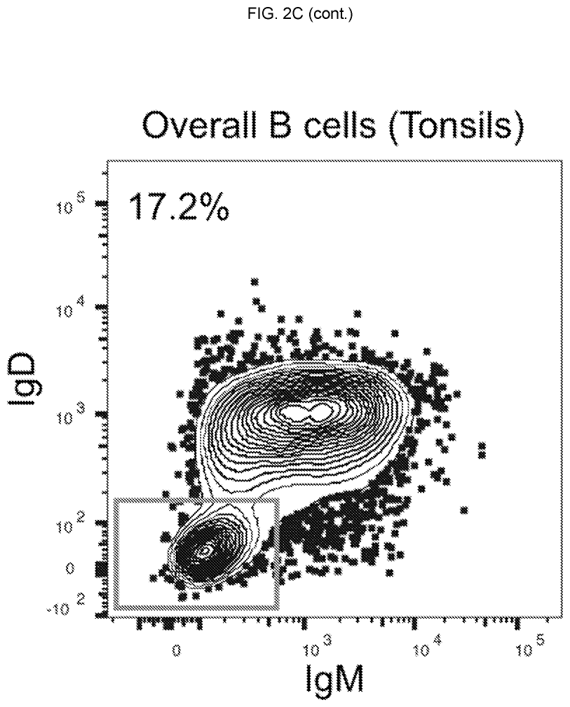

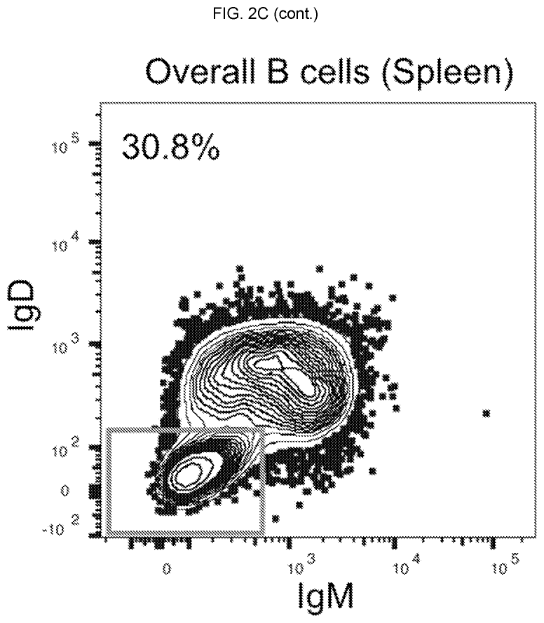

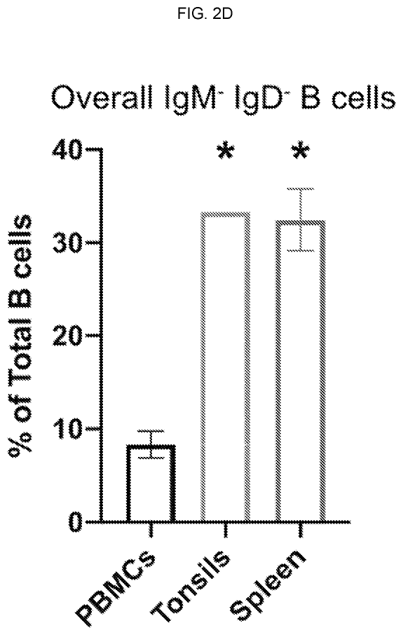

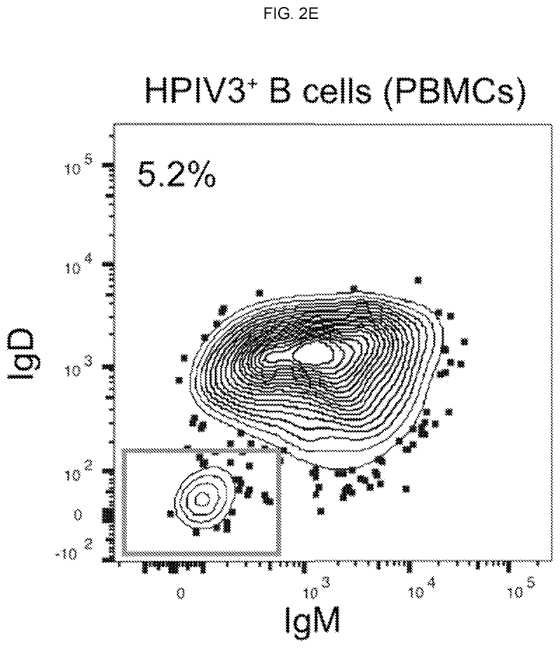

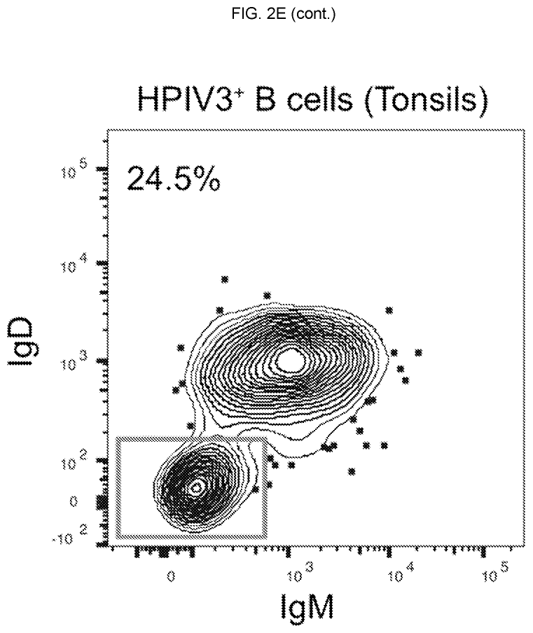

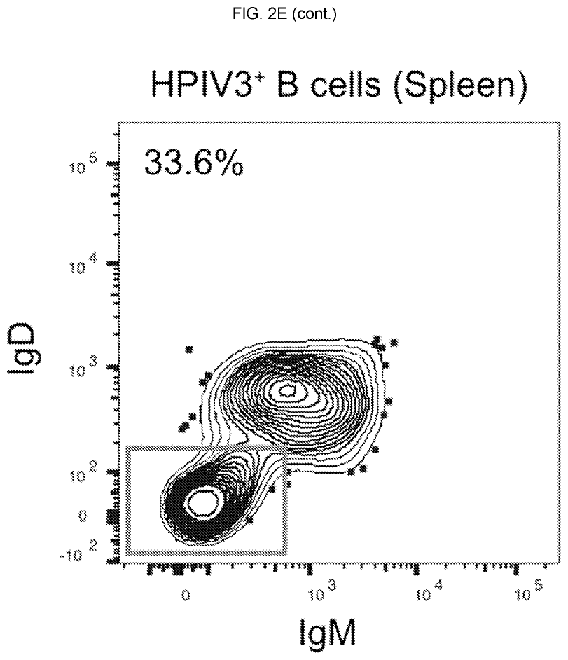

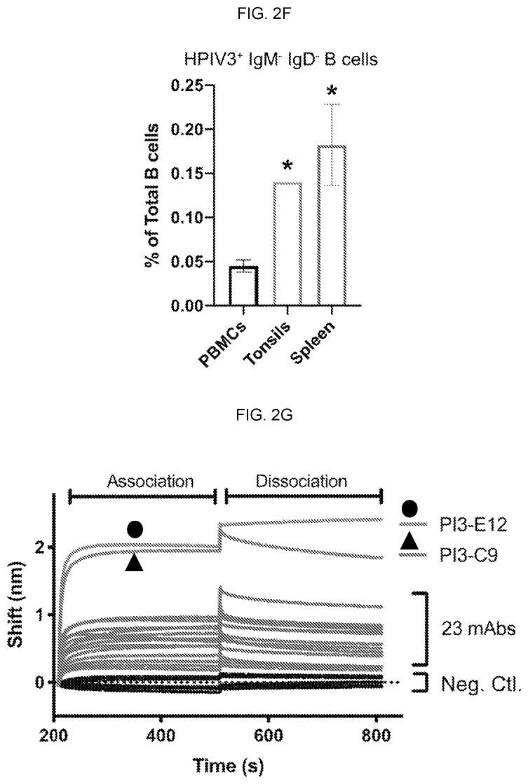

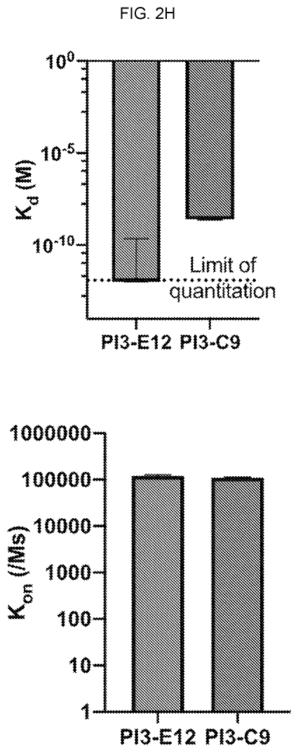

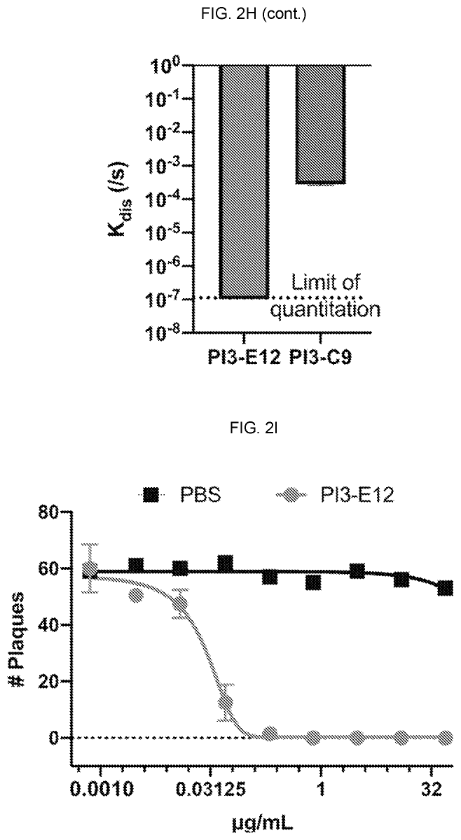

Using this approach, B cells that bound the preF conformation and not the postF conformation were identified with fluorochrome-labeled streptavidin using flow cytometry (FIG. 2A). Since the seroprevalence to HPIV3 in humans is almost complete (Henrickson, Clin Microbiol Rev 16, 242-264, (2003)), a pre-screen of donors for sero-positivity against HPIV3 was not needed. This approach was used to assess samples of peripheral blood, tonsils and spleens from unmatched donors since these secondary lymphoid organs might be enriched for B cells that had undergone affinity maturation for binding HPIV3. Overall, 0.20-1.75% of B cells in these tissues bound HPVI3 preF (FIG. 2B). The overall frequency of IgM⁻ IgD⁻ isotype switched B cells was increased in the spleen and tonsils (FIGS. 2C & 2D), with an increase detected within the HPIV3 preF⁺ population that reached statistical significance in tonsils and spleen (FIGS. 2E & 2F).

Binding and neutralization of HPIV3 within the human B cell repertoire. It was next sought to determine how many of the HPIV3 preF-specific B cells identified by flow cytometry produced antibodies that could bind and neutralize HPIV3. For this 92 HPIV3 preF-specific B cells were sorted from PBMCs from two donors into individual wells of a 96-well plate and obtained 25 paired heavy and light chain sequences (Table 1).

TABLE 1

| | Heavy chain | Kappa chain | Lambda chain |
|---|---|---|---|
| mAb | gene | gene | gene |
| PI3-A2 | IGHV3-15*01 | | IGLV2-14*01 |
| PI3-A3 | IGHV3-11*03 | IGKV3-15*01 | |
| PI3-A10 | IGHV4-31*03 | | IGLV2-14*01 |
| PI3-A12K | IGHV4-39*01 | IGKV4-1*01 | |
| PI3-A12L | IGHV4-39*01 | | IGLV4-69*01 |
| PI3-B1 | IGHV4-59*01 | IGKV3-15*01 | |
| PI3-B7 | IGHV1-18*01 | IGKV3-20*01 | |
| PI3-B8 | IGHV3-21*01 | | IGLV1-40*01 |
| PI3-B9 | IGHV3-20*01 | IGKV1-8*01 | |
| PI3-B10 | IGHV5-51*03 | IGKV2-28*01 | |
| PI3-B11 | IGHV3-21*02 | IGKV1-6*01 | |
| PI3-C8 | IGHV3-15*01 | | IGLV3-1*01 |
| PI3-C9 | IGHV3-11*03 | | IGLV2-14*01 |
| PI3-C10 | IGHV3-64D*06 | IGKV2-28*01 | |
| PI3-D2 | IGHV3-11*03 | | IGLV1-44*01 |
| PI3-D5 | IGHV3-20*01 | | IGLV3-25*02 |
| PI3-E2 | IGHV3-66*01 | IGKV1-39*01 | |
| PI3-E6 | IGHV5-51*03 | IGKV3-15*01 | |
| PI3-E8 | IGHV3-20*01 | | IGLV1-40*01 |
| PI3-E9 | IGHV3-20*01 | | IGLV2-14*01 |
| PI3-E12 | IGHV3-11*03 | IGKV2-28*01 | |
| PI3-F6 | IGHV3-11*03 | | IGLV2-14*01 |
| PI3-F11 | IGHV3-23*01 | IGKV4-1*01 | |
| PI3-F12 | IGHV3-15*01 | | IGLV7-46*01 |
| PI3-G7 | IGHV5-51*01 | IGKV1-5*03 | |
| PI3-G9 | IGHV3-23*01 | | IGLV2-11*01 |

From these, all 25 were cloned as monoclonal antibodies, tested for binding by biolayer interferometry (BLI), and binding to purified HPIV3 preF for 100% (25/25) (FIG. 2G) confirmed. Amongst this group, two monoclonal antibodies named PI3-E12 and PI3-C9 bound with high affinities (K_{D}) to HPIV3 preF, 1×10⁻¹² M and 2.5×10⁻⁹ M, respectively (FIG. 2H). However, an analysis of binding kinetics revealed that PI3-E12 had a higher affinity than PI3-C9 for HPIV3 preF due to slower dissociation, 1×10⁻⁷/s versus 2.7×10⁻⁴/s, respectively (FIG. 2H). Correspondingly, PI3-E12, but not PI3-C9, was capable of neutralizing live virus in vitro (FIG. 2I). Together, these data indicated that a low frequency of HPIV3 preF tetramer-binding B cells express high affinity antibodies capable of neutralizing HPIV3.

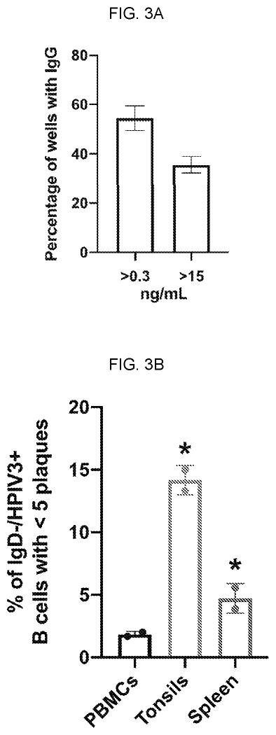

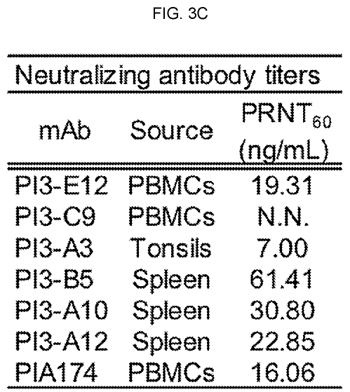

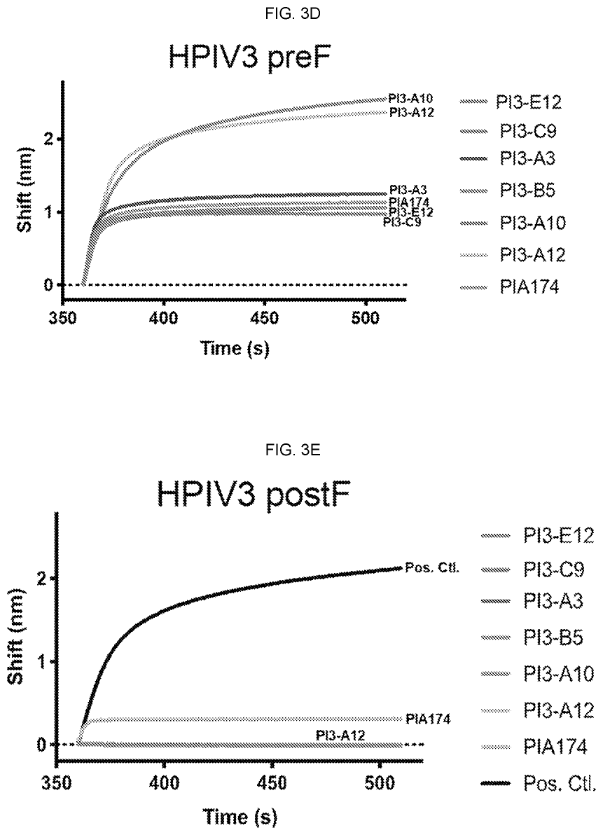

To focus upon B cells producing neutralizing antibodies, the assay was modified to sort individual B cells onto irradiated CD40L/IL2/IL21-expressing 3T3 feeder cells. The stimulation of antibody secretion enabled higher-throughput screening of culture supernatants for neutralization prior to antibody cloning. In general, over half of sorted B cells produced detectable antibody by ELISA (FIG. 3A). This assay was applied to stimulate single HPIV3 preF-specific B cells and exclude IgD-expressing cells since these cells would be the least likely to have undergone the somatic hypermutation and affinity maturation necessary for potent neutralization. Using this approach, 14% of IgD⁻ HPIV3 pre-F-specific B cells sorted from tonsils produced HPIV3 neutralizing antibodies as compared to 5% from the spleen and 2% from peripheral blood (2%) (FIG. 3B).

Using the described new screening method, four additional HPIV3 neutralizing monoclonal antibodies named PI3-A3, PI3-B5, PI3-A10, and PI3-A12 were sequenced, cloned, and produced (FIG. 3C). The neutralization potency of these antibodies ranged from 7.0 to 61.4 ng/mL (FIG. 3C). Each neutralizing monoclonal antibody used different immunoglobulin heavy chain alleles (Table 2).

TABLE 2

| | | % Similarity | | | % Similarity |
|---|---|---|---|---|---|
| | Heavy | to Heavy | Kappa | Lambda | to Light |
| | Chain | Chain | Chain | Chain | Chain |
| mAb | Gene | Germ-line | Gene | Gene | Germ-line |
| PI3-E12 | 3-11*03 | 93.8 | 2-28*01 | | 96.9 |
| PI3-C9 | 3-11*03 | 96.5 | | 2-14*01 | 98.6 |
| PI3-A3 | 3-7*01 | 94.8 | 1-5*03 | | 95.7 |
| PI3-B5 | 5-51*01 | 96.5 | 1-5*03 | | 95 |
| PI3-A10 | 3-30*03 | 90.3 | 3-11*01 | | 94.3 |
| PI3-A12 | 4-30-4*01 | 90.4 | | 1-47*01 | 90.5 |
| PIA174 | 4-59 | 92.7 | 1-39 | | 92.5 |

Each also used different immunoglobulin light chain alleles, except for PI3-A3 and PI3-B5 which both utilized the kappa allele 1-5*03. None of the alleles matched that of a previously described HPIV3 antibody PIA174. Stewart-Jones, et al. Proc Natl Acad Sci USA 115, 12265-12270, (2018). The similarity to germ-line sequences of the variable genes from these neutralizing antibodies ranged from 90-97% (Table 2). All of the newly described antibodies in this study bound strongly to the preF conformation without any detectable binding to the postF conformation (FIGS. 3D & 3E). The previously described antibody PIA174 similarly bound strongly to HPIV3 preF but also bound weakly to the postfusion conformation (FIGS. 3D & 3E). In anticipation of administering these antibodies in vivo, it was confirmed that none of the antibodies were found to bind permeabilized HEp-2 cells (FIG. 3F), a common assessment of autoreactivity (Steach, et al. J Immunol 204, 498-509, (2020); Bancroft, et al. J Exp Med 216, 2331-2347, (2019)).

Cross-competition binding experiments were next performed to gauge the antigenic sites on HPIV3 preF allowing for neutralization. Two of these four new neutralizing monoclonal antibodies (PI3-A3 and -B5) fully competed with each other and the previously described antibody PIA174 (FIG. 12A). PI3-A10 also competed with this group, but only partially with PI3-E12 (FIG. 12A). Based on the known binding site of PIA174, this antigenic site is likely located at the apex of HPIV3 preF (Stewart-Jones, et al. Proc Natl Acad Sci USA 115, 12265-12270, (2018)).

This antigenic site is referred to as 0 on HPIV3 preF for consistency since the apices of RSV and HMPV preF are called antigenic site 0 (McLellan, et al. Science 340, 1113-1117, (2013); Battles, et al. Nat Commun 8, 1528, (2017)).

The fourth neutralizing monoclonal, PI3-A12, only weakly competed with PI3-A10, suggesting the presence of an antigenic site vulnerable to neutralization by antibodies outside of antigenic site Ø (FIG. 12A).

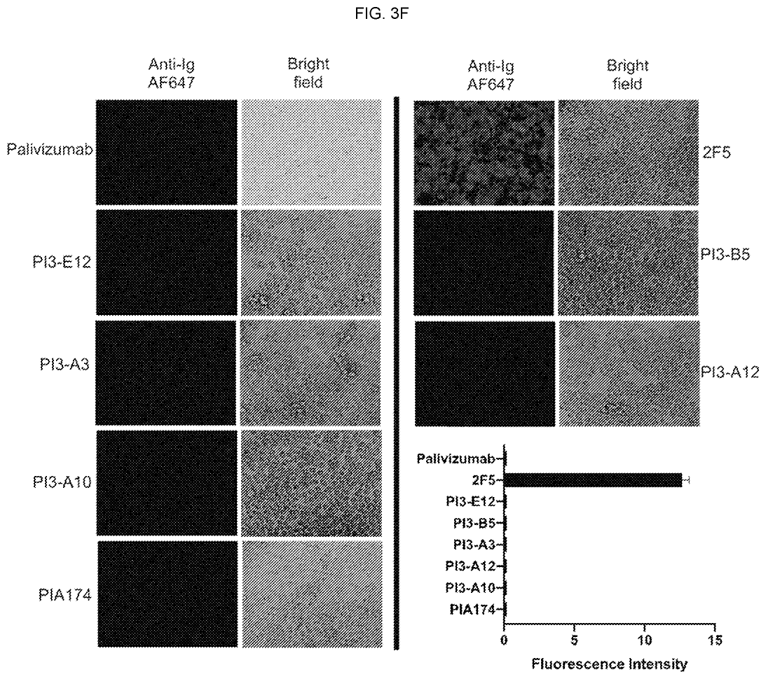

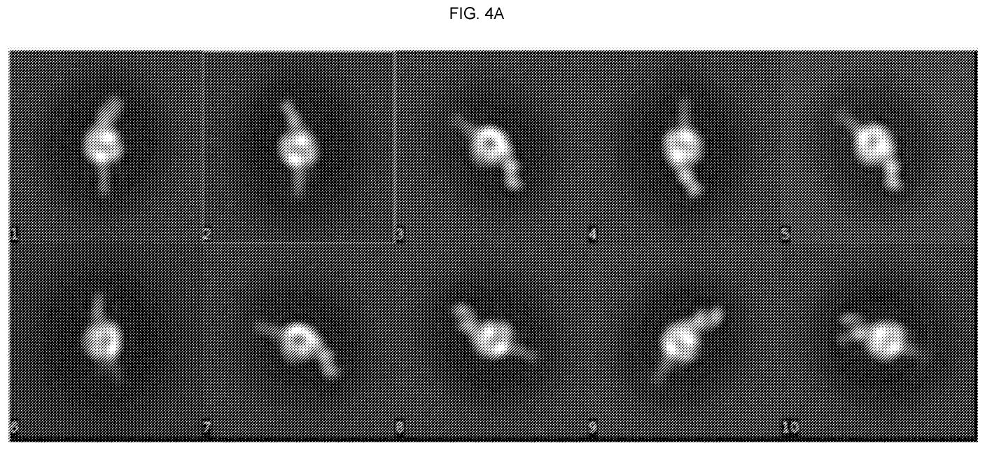

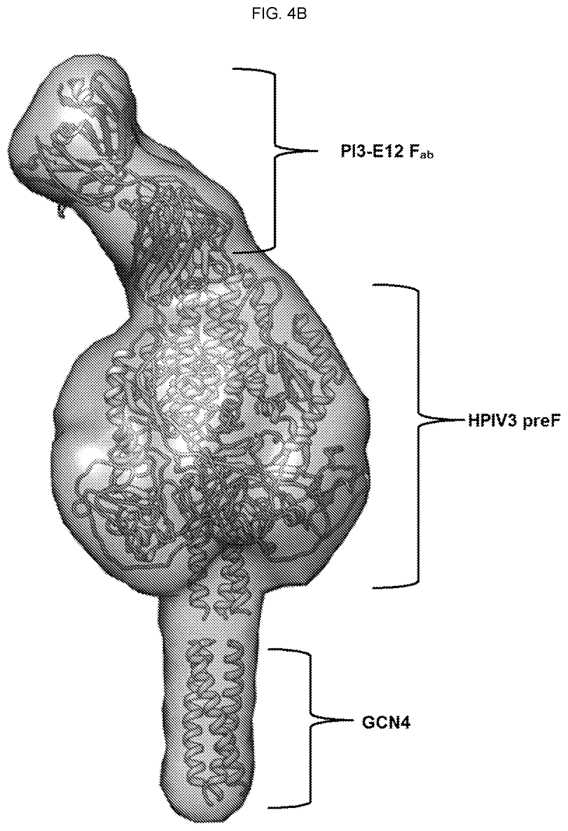

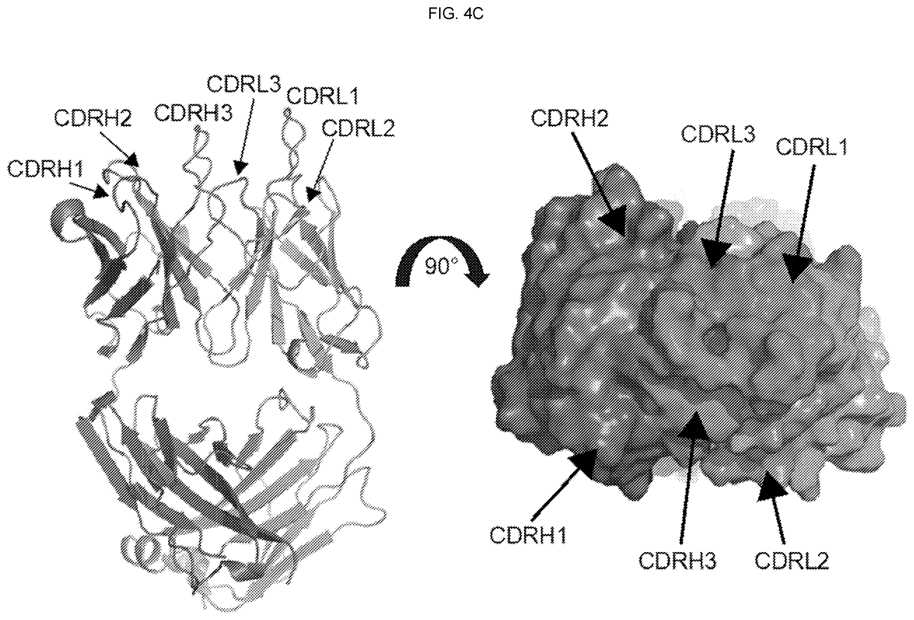

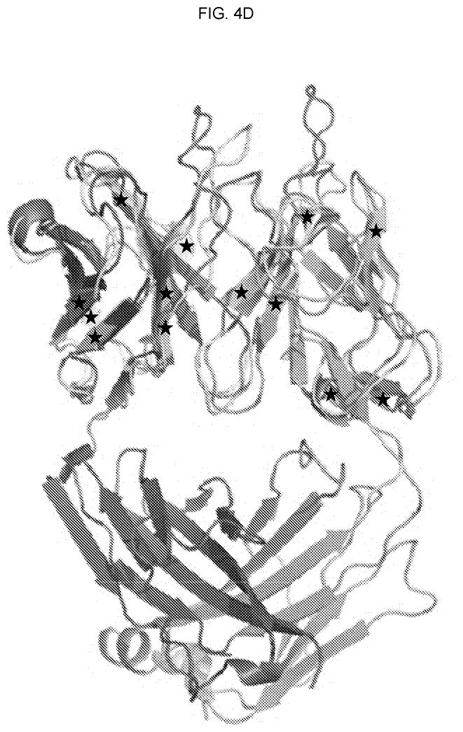

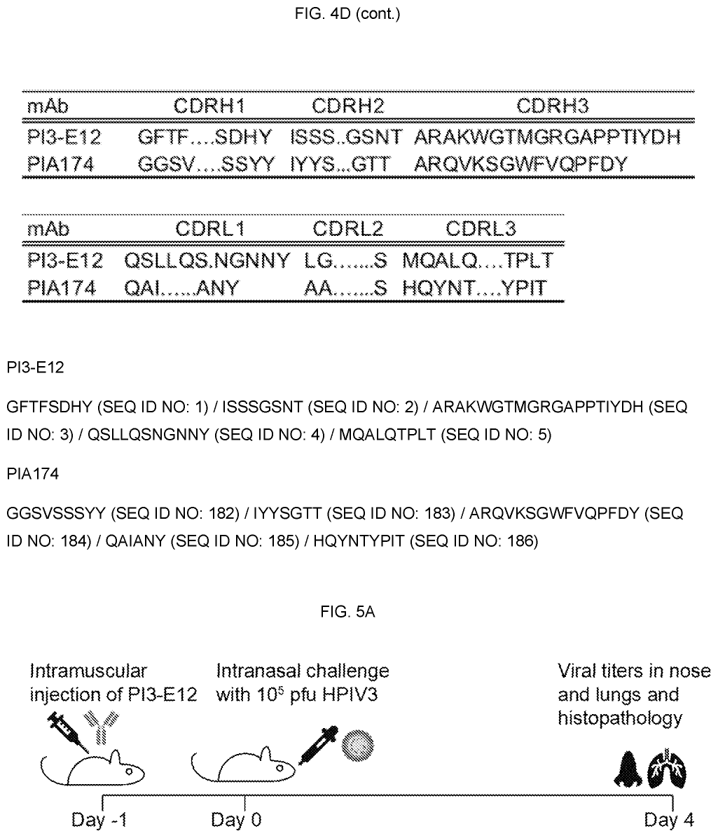

Since PI3-E12 appeared to bind the HPIV3 preF apical antigenic site 0 differently than the previously described PIA174, negative stain electron microscopy of PI3-E12 in complex with HPIV3 preF and crystallization of PI3-E12 alone was performed. Negative stain electron microscopy (nsEM) was used to obtain a low-resolution 3D reconstruction of PI3-E12 F_{ab} in complex with HPIV3 preF. As predicted earlier, the PI3-E12 F_{ab} bound in a 1:1 ratio (F_{ab}:trimer) at the apex of HPIV3 preF. However, PI3-E12 F_{ab} appears to bind HPIV3 preF with a different angle of approach compared to PIA174 (FIGS. 4A & 4B). A 2.1 Å structure of PI3-E12 F_{ab} using X-ray crystallography was obtained (FIGS. 4B & 4C & Table 3).

TABLE 3
Data collection and refinement statistics for PI3-E12 Fab

| | PI3-E12 Fab |
|---|---|
| Data collection | |
| Space group | P2₁2₁2₁ |
| Cell dimensions | |
| a, b, c (Å) | 51.031, 79.924, 99.685 |
| α, β, γ (□) | 90, 90, 90 |
| Resolution (Å) | 50-2.09 (2.13-2.09)* |
| R_{sym} or R_{merge} | 0.122 (0.404)* |
| //s/ | 16.01 (3.06)* |
| Completeness (%) | 98.0 (83.8)* |
| Redundancy | 6.5 (4.8)* |
| CC_{1/2} | 0.997 (0.905)* |
| Refinement | |
| Resolution (Å) | 23.62-2.09 (2.154-2.09)* |
| No. reflections | 24469 (2026)* |
| R_{work}/R_{free} | 19.60/24.97 (23.83/34.01)* |
| No. atoms | 3729 |
| Protein | 3333 |
| Water | 338 |
| Ligand | 58 |
| B-factors (Å²) | 30.24 |
| Protein | 29.42 |
| Water | 33.96 |
| Ligand | 55.66 |
| R.m.s deviations | |
| Bond lengths (Å) | 0.002 |
| Bond angles (°) | 0.51 |
| Ramachadran Favored % | 97.46 |
| Ramachadran Outliers % | 0.23 |
| MolProbity all-atoms clashscore | 2.24 |
| PDB ID | 6WRP |

*Statistics for the highest-resolution shell are shown in parentheses.

The structure of PI3-E12 F_{ab} and PIA174 were superimposed (root mean standard deviation: 1.4 A2) and the main differences in structural organization lay within the CDRL1 which is longer in PI3-E12 and CDRH3 (FIG. 4D).

Together, the results indicate that the HPIV3 preF apical antigenic site 0 is a common target of neutralizing antibodies that can be accessed by antibodies using different gene segments and with different angles of approach.

In vivo protection against HPIV3 infection. The clinical utility of PI3-E12 in an animal challenge model of HPIV3 infection was next investigated. Although the human parainfluenza viruses do not replicate in mice, lower respiratory tract pathology and viral replication can be demonstrated in cotton rats infected intranasally with HPIV3. Henrickson, Clin Microbiol Rev 16, 242-264, (2003); Ottolini, et al. J Gen Virol 77 (Pt 8), 1739-1743, (1996).

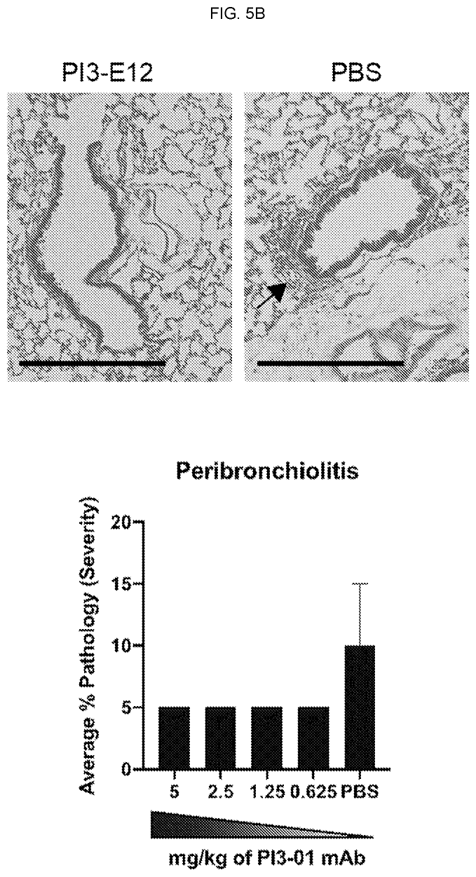

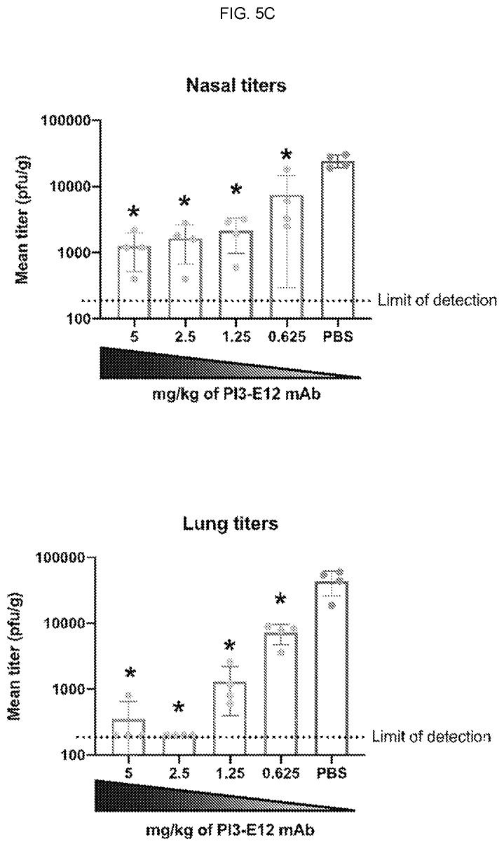

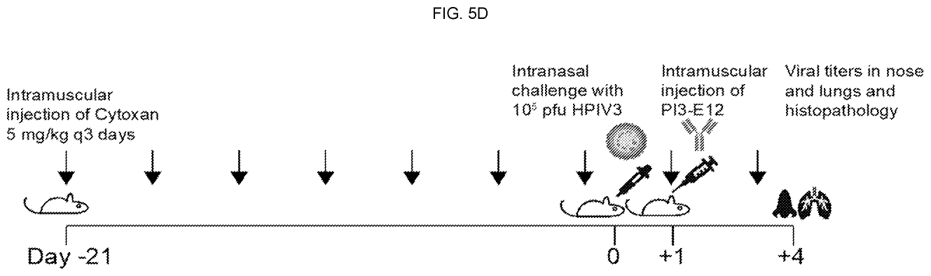

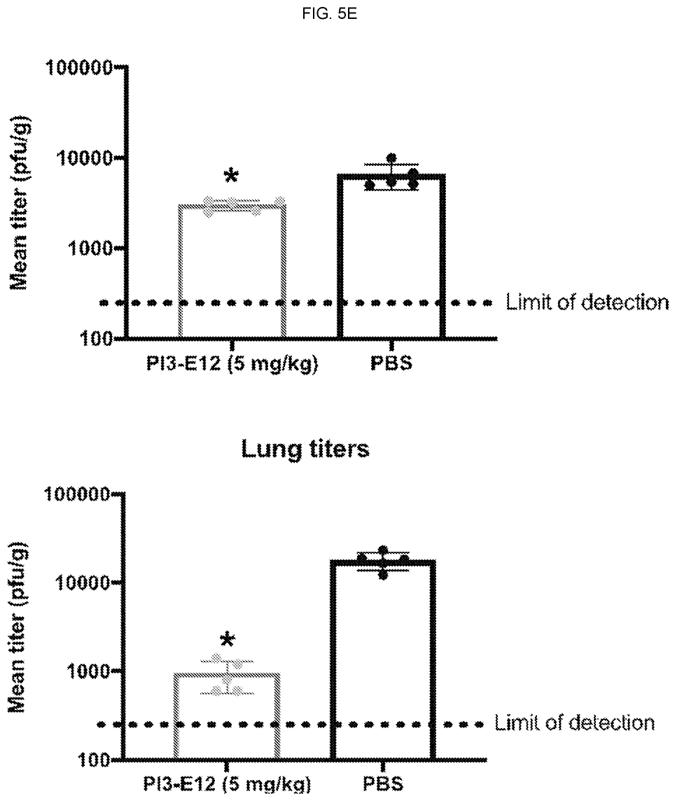

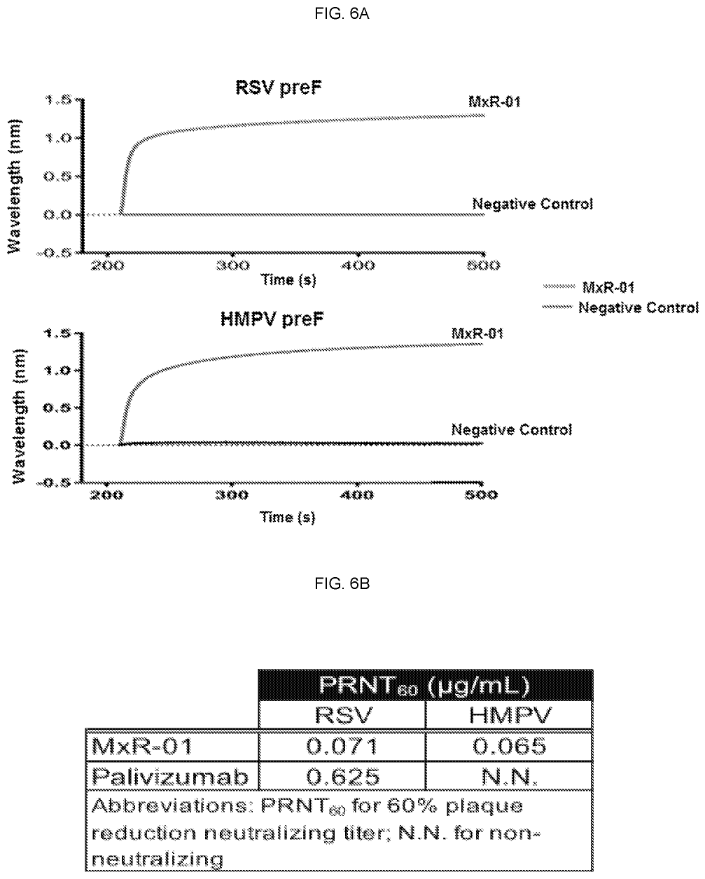

The cotton rat model was used in the past to predict not only the efficacy of antibody immunoprophylaxis but also the exact dose of palivizumab, 15 mg/kg, that would be effective against RSV in human infants (Johnson, et al. J Infect Dis 176, 1215-1224, (1997)). Therefore, a similar experimental design was adopted. 0.625-5 mg/kg of PI3-E12 was injected intramuscularly one day prior to intranasal infection of cotton rats with 10⁵ pfu of HPIV3 (FIG. 5A). None of the animals that received PI3-E12 developed peribronchiolitis, whereas peribronchiolitis could be detected 4 days after infection in animals that received PBS (FIG. 5B). Consistent with decreased peribronchiolitis, the amount of HPIV3 detected in the lung was reduced 6-fold at this low dose of PI3-E12 and was below the limit of detection in 8/9 animals injected with 2.5 mg/kg or more (FIG. 5C). More modest reductions in HPIV3 replication in the nose was also detected (FIG. 5C), which was expected given the relatively poor ability of IgG antibodies entering this compartment (Kirkeby, et al. Clin Diagn Lab Immunol 7, 31-39, (2000); Fisher, et al. J Infect Dis 180, 1324-1327, (1999)). Together, the data indicate that an EC₅₀ of 0.35 mg/kg and EC₉₉ of 1.80 mg/kg for PI3-E12-mediated prevention of HPIV3 in the lungs.

Since patients receiving cytotoxic therapy for cancer or autoimmune diseases and other immunocompromised groups are at the highest risk for severe disease and mortality due to HPIV3 infection, the efficacy of PI3-E12 as treatment in immunosuppressed animals was tested. For this, a similar experimental design used to model RSV in immunocompromised cotton rats was adopted (Boukhvalova, et al. Bone Marrow Transplant 51, 119-126, (2016); Johnson, et al. Infect Immun 37, 369-373 (1982); Ottolini, et al. Bone Marrow Transplant 29, 117-120, (2002)).

Animals were treated with 5 mg/kg of Cytoxan injected every three days for three weeks prior to intranasal infection with 10⁵ pfu of HPIV3 (FIG. 5D). Five days after infection, 104 pfu/g could be detected in the lungs and nose of control animals that did not receive PI3-E12 (FIG. 5E). In contrast, viral titers were diminished 28-fold in the lungs and 2-fold in the nose when 5 mg/kg of PI3-E12 was injected one day after infection (FIG. 5E).

Together, the data indicates that PI3-E12 can both prevent and treat of HPIV3 infection.

Materials & Methods. Study design. The size of experimental groups is specified in figure legends. Peripheral blood was obtained by venipuncture from healthy, HIV-seronegative adult volunteers enrolled in the Seattle Area Control study, which was approved by the controlling institutional review board. PBMCs were isolated from whole blood using Accuspin System Histopaque-1077 (Sigma-Aldrich).

Institutional review board approval for studies involving human tonsils was obtained from Seattle Children's Hospital. Studies involving human spleens were deemed non-human subjects research since tissue was de-identified, otherwise discarded, and originated from deceased individuals. Tissue fragments were passed through a basket screen, centrifuged at 1400 RPM for 7 minutes, incubated with ACK lysis buffer (Thermo Fisher) for 3.5 minutes, resuspended in RPMI (Gibco), and passed through a stacked 500 μm and 70 μm cell strainer. Cells were resuspended in 10% dimethylsulfoxide in heat-inactivated fetal calf serum (Gibco) and cryopreserved in liquid nitrogen before use.

Cell lines. 293F cells (Thermo Fisher) were cultured in Freestyle 293 media (Thermo Fisher). Vero cells (ATCC CCL-81), LLC-MK2 cells (ATCC CCL-7.1), and HEp-2 (ATCC CCL-23) were cultured in DMEM (Gibco) supplemented with 10% fetal calf serum and 100 U/ml penicillin plus 100 μg/mL streptomycin (Gibco). 3T3⁻CD40L/IL2/IL21 feeder cells were cultured in DMEM supplemented with 10% fetal calf serum, penicillin and streptomycin, plus 0.4 mg/mL geneticin. Irradiation was performed with 5,000 rads.

Viruses. Wild-type rHPIV3 was a recombinant version of strain JS (GenBank accession number Z11575) and modified as previously described to express enhanced GFP. Liu, et al. PLoS One 15, e0228572, (2020). Virus was cultured on LLC-MK2 cells and subsequently purified by centrifugation in a discontinuous 30%/60% sucrose gradient with 0.05 M HEPES and 0.1 M MgSO₄ (Sigma-Aldrich) at 120,000×g for 90 min at 4° C. Virus titers were determined by infecting Vero cell monolayers in 24-well plates with serial 10-fold dilutions of virus, overlaying with DMEM containing 4% methylcellulose (Sigma-Aldrich), and counting fluorescent plaques using a Typhoon scanner at five days post-infection (GE Life Sciences).

Expression and purification of antigens. Expression plasmids for His-tagged HPIV3 preF and postF antigens are described in Stewart-Jones, et al. Proc Natl Acad Sci USA 115, 12265-12270, (2018). 293F cells were transfected at a density of 10⁶ cells/mL in Freestyle 293 media using 1 mg/mL PEI Max (Polysciences). Transfected cells were cultured for 7 days with gentle shaking at 37° C. Supernatant was collected by centrifuging cultures at 2,500 g for 30 minutes followed by filtration through a 0.2 μM filter. The clarified supernatant was incubated with Ni Sepharose beads overnight at 4° C., followed by washing with wash buffer containing 50 mM Tris, 300 mM NaCl, and 8 mM imidazole. His-tagged protein was eluted with an elution buffer containing 25 mM Tris, 150 mM NaCl, and 500 mM imidazole. The purified protein was run over a 10/300 Superose 6 size-exclusion column (GE Life Sciences). Fractions containing the trimeric HPIV3 F proteins were pooled and concentrated by centrifugation in an Amicon ultrafiltration unit (Millipore) with a 50 kDa molecular weight cut-off. Two units of biotinylated thrombin (Millipore) was mixed with each 1 mg of protein overnight to cleave off tags, streptavidin agarose (Millipore) was added for another hour to remove thrombin and the cleaved tags, and the mixture was centrifuged through a PVDF filter (Millipore) to remove the streptavidin agarose. The concentrated sample was stored in 50% glycerol at −20° C.

Tetramerization of antigens. Purified HPIV3 F was biotinylated using an EZ-link Sulfo-NHS-LC-Biotinylation kit (Thermo Fisher) using a 1:1.3 molar ratio of biotin to F. Unconjugated biotin was removed by centrifugation using a 50 kDa Amicon Ultra size exclusion column (Millipore). To determine the average number of biotin molecules bound to each molecule of F, streptavidin-PE (ProZyme) was titrated into a fixed amount of biotinylated F at increasing concentrations and incubated at room temperature for 30 min. Samples were run on an SDS-PAGE gel (Invitrogen), transferred to nitrocellulose, and incubated with streptavidin-Alexa Fluor 680 (Thermo Fisher) at a dilution of 1:10,000 to determine the point at which there was excess biotin available for the streptavidin-Alexa Fluor 680 reagent to bind. Biotinylated F was mixed with streptavidin-APC at the ratio determined above to fully saturate streptavidin and incubated for 30 min at room temperature. Unconjugated F was removed by centrifugation using a 300K Nanosep centrifugal device (Pall Corporation). APC/DyLight 755 tetramers were created by mixing F with streptavidin-APC pre-conjugated with DyLight 755 (Thermo Fisher) following the manufacturer's instructions. On average, APC/DyLight 755 contained 4-8 DyLight molecules per APC. The concentration of each F tetramer was calculated by measuring the absorbance of APC (650 nm, extinction coefficient=0.7 μM⁻¹ cm⁻¹).

Tetramer enrichment. 100-200×10⁶ frozen PBMCs, 20-50×10⁶ frozen tonsil cells, or 40-80×10⁶ frozen spleen cells were thawed into DMEM with 10% fetal calf serum and 100 U/ml penicillin plus 100 μg/ml streptomycin. Cells were centrifuged and resuspended 50 μL of ice-cold FACS buffer composed of PBS and 1% newborn calf serum (Thermo Fisher). APC/DyLight 755 conjugated tetramers were added at a final concentration of 25 nM in the presence of 2% rat and mouse serum (Thermo Fisher) and incubated at room temperature for 10 min. APC tetramers were next added at a final concentration of 5 nM and incubated on ice for 25 min, followed by a 10-mL wash with ice-cold FACS buffer. 50 μL of both anti-APC-conjugated microbeads (Miltenyi Biotec) were added and incubated on ice for 30 min. 3 mL of FACS buffer was then added and the mixture was passed over a magnetized LS column (Miltenyi Biotec). The column was washed once with 5 mL ice-cold FACS buffer and then removed from the magnetic field. 5 mL ice-cold FACS buffer was pushed through the unmagnetized column twice using a plunger to elute the bound cell fraction.

Flow cytometry. Cells were incubated in 50 μL of FACS buffer containing a cocktail of antibodies for 30 minutes on ice prior to washing and analysis on a FACS Aria (BD). Antibodies included anti-IgM FITC (G20-127, BD), anti-CD19 BUV395 (SJ25C1, BD), anti-CD3 BV711 (UCHT1, BD), anti-CD14 BV711 (MOP-9, BD), anti-CD16 BV711 (3G8, BD), anti-CD20 BUV737 (2H7, BD), anti-IgD BV605 (IA6-2, BD), and a fixable viability dye (FV). Absolute counts within each specimen were calculated by adding a known amount of AccuCheck Counting Beads (Thermo Fisher). B cells were individually sorted into either 1) empty 96-well PCR plates and immediately frozen or 2) into flat-bottom 96 well plates containing feeder cells that had been seeded at a density of 28,600 cells/well one day prior in 100 μL of IMDM media (Gibco) containing 10% fetal calf serum, 100 U/ml penicillin plus 100 μg/ml streptomycin, and 2.5 μg/mL amphotericin. For B cells sorted onto feeder cells were cultured at 37° C. for 13 days.

ELISA. Nunc maxsorp 96-well plates (Thermo Fisher) were coated with 100 ng of goat anti-human F_{ab} (Jackson ImmunoResearch) for 90 minutes at 4° C. Wells were washed three times with PBS and then blocked with PBS containing 1% bovine serum albumin (Sigma-Aldrich) for one hour at room temperature. Antigen coated plates were incubated with culture supernatants for 90 minutes at 4° C. A standard curve was generated with serial two-fold dilutions of palivizumab. Wells were washed three times with PBS followed by a one-hour incubation with horse radish peroxidase-conjugated goat anti-human total Ig at a dilution of 1:6000 (Invitrogen). Wells were then washed four times with PBS followed by a 5-15 minute incubation with TMB substrate (SeraCare). Absorbance was measured at 405 nm using a Softmax Pro plate reader (Molecular Devices). The concentration of antibody in each sample was determined by reference to the standard curve and dilution factor.

Neutralization assays. For neutralization screening of culture supernatants, Vero cells were seeded in 96-well flat bottom plates and cultured for 48 hours. After 13 days of culture, 40 μL of culture supernatant was mixed with 25 μL of sucrose purified GFP-HPIV3 diluted to 2,000 plaque forming units (pfu)/mL for one hour at 37° C. Vero cells were incubated with 50 μL of the supernatant/virus mixture for one hour at 37° C. to allow viral adsorption. Each well was then overlaid with 100 μL DMEM containing 4% methylcellulose. Fluorescent plaques were counted at five days post-infection using a Typhoon imager. Titers of HPIV3-specific monoclonal antibodies were determined by a 60% plaque reduction neutralization test (PRNT₆₀). Vero cells were seeded in 24-well plates and cultured for 48 hours. Monoclonal antibodies were serially diluted 1:4 in 120 μL DMEM and mixed with 120 μL of sucrose purified HPIV3 diluted to 2,000 pfu/mL for one hour at 37° C. Vero cells were incubated with 100 μL of the antibody/virus mixture for one hour at 37° C. to allow viral adsorption. Each well was then overlaid with 500 μL DMEM containing 4% methylcellulose. Fluorescent plaques were counted at five days post-infection using a Typhoon imager. PRNT₆₀ titers were calculated by linear regression analysis.

B cell receptor sequencing and cloning. For individual B cells sorted and frozen into empty 96-well PCR plates, reverse transcription (RT) was directly performed after thawing plates using SuperScript IV (Thermo Fisher) as previously described. Wu, et al. Science 329, 856-861, (2010); Tiller, et al. J Immunol Methods 329, 112-124, (2008).

Briefly, 3 μL RT reaction mix consisting of 3 μL 50 μM random hexamers (Thermo Fisher), 0.8 μL of 25 mM deoxyribonucleotide triphosphates (dNTPs; Thermo Fisher), 1 μL (20 U) SuperScript IV RT, 0.5 μL (20 U) RNaseOUT (Thermo Fisher), 0.6 μL of 10% Igepal (Sigma-Aldrich), and 15 μL RNase free water was added to each well containing a single sorted B cell and incubated at 50° C. for 1 hour. For individual B cells sorted onto feeder cells, supernatant was removed after 13 days of culture, plates were immediately frozen on dry ice, stored at −80° C., thawed, and RNA was extracted using the RNeasy Micro Kit (Qiagen). The entire eluate from the RNA extraction was used instead of water in the RT reaction. Following RT, 2 μL of cDNA was added to 19 μl PCR reaction mix so that the final reaction contained 0.2 μL (0.5 U) HotStarTaq Polymerase (Qiagen), 0.075 μL of 50 μM 3′ reverse primers, 0.115 μL of 50 μM 5′ forward primers, 0.24 μL of 25 mM dNTPs, 1.9 μL of 10× buffer (Qiagen), and 16.5 μL of water. The PCR program was 50 cycles of 94° C. for 30 s, 57° C. for 30 s, and 72° C. for 55 s followed by 72° C. for 10 min for heavy and kappa light chains. The PCR program was 50 cycles of 94° C. for 30 s, 60° C. for 30 s, and 72° C. for 55 s followed by 72° C. for 10 min for lambda light chains. After the first round of PCR, 2 μL of the PCR product was added to 19 μL of the second-round PCR reaction so that the final reaction contained 0.2 μL (0.5 U) HotStarTaq Polymerase, 0.075 μL of 50 μM 3′ reverse primers, 0.075 μL of 50 μM 5′ forward primers, 0.24 μL of 25 mM dNTPs, 1.9 μL 10× buffer, and 16.5 μL of water. PCR programs were the same as the first round of PCR. 4 μL of the PCR product was run on an agarose gel to confirm the presence of a 500-bp heavy chain band or 450-bp light chain band. 5 μL from the PCR reactions showing the presence of heavy or light chain amplicons was mixed with 2 μL of ExoSAP-IT (Thermo Fisher) and incubated at 37° C. for 15 min followed by 80° C. for 15 min to hydrolyze excess primers and nucleotides. Hydrolyzed second-round PCR products were sequenced by Genewiz with the respective reverse primer used in the 2^{nd} round PCR, and sequences were analyzed using IMGT/V-Quest to identify V, D, and J gene segments. Paired heavy chain VDJ and light chain VJ sequences were cloned into pTT3-derived expression vectors containing the human IgG1, IgK, or IgL constant regions using In-Fusion cloning (Clontech) as described in McGuire, et al. Nat Commun 7, 10618, (2016).

Monoclonal antibody production. Secretory IgG was produced by co-transfecting 293F cells at a density of 10⁶ cells/mL with the paired heavy and light chain expression plasmids at a ratio of 1:1 in Freestyle 293 media using 1 mg/mL PEI Max. Transfected cells were cultured for 7 days with gentle shaking at 37° C. Supernatant was collected by centrifuging cultures at 2,500 g for 15 minutes followed by filtration through a 0.2 μM filter. Clarified supernatants were then incubated with Protein A Agarose (Thermo Scientific) followed by washing with IgG binding buffer (Thermo Scientific). Antibodies were eluted with IgG Elution Buffer (Thermo Scientific) into a neutralization buffer containing 1 M Tris-base pH 9.0. Purified antibody was concentrated and buffer exchanged into DBPS using an Amicon ultrafiltration unit with a 50 kDa molecular weight cut-off.

Biolayer interferometry. BLI assays were performed on the Octet.Red instrument (ForteBio). Binding assays were performed at room temperature with shaking at 500 rpm. Anti-human IgG capture sensors (ForteBio) were loaded in kinetics buffer (PBS with 0.01% BSA, 0.02% Tween 20, and 0.005% NaN₃, pH 7.4) containing 40 μg/mL purified monoclonal antibody for 150 s. After loading, the baseline signal was recorded for 60 s in kinetics buffer. The sensors were then immersed in kinetics buffer containing 1 μM purified HPIV3 F for a 300 s association step followed by immersion in kinetics buffer for an additional 300 s dissociation phase. The maximum response was determined by averaging the nanometer shift over the last 5 s of the association step after subtracting the background signal from each analyte-containing well using a negative control monoclonal antibody at each time point. Curve fitting was performed using a 1:1 binding model and ForteBio data analysis software. For competitive binding assays, penta-His capture sensors (ForteBio) were loaded in kinetics buffer containing 1 μM His-tagged HPIV3 F for 300 s. After loading the baseline signal was recorded for 30 s in kinetics buffer. The sensors were then immersed for 300 s in kinetics buffer containing 40 μg/mL of the first antibody followed by immersion for another 300 s in kinetics buffer containing 40 μg/mL of the second antibody. Percent competition was determined by dividing the maximum increase in signal of the second antibody in the presence of the first antibody by the maximum signal of the second antibody alone.

Auto-reactivity assay. HEp-2 cells were seeded into 96-well plates at a density of 50,000 cells/well one day prior to fixation with 50% acetone and 50% methanol for 10 minutes at −20° C. Cells were then permeabilized and blocked with PBS containing 1% triton (Sigma-Aldrich) and 1% bovine serum albumin for 30 minutes at room temperature. 100 μL of each monoclonal antibody at 0.1 mg/mL was added for 30 min at room temperature. The 2F5 positive control was obtained from the NIH AIDS Reagent Program. Wells were then washed four times in PBS followed by incubation with oat anti-human IgG Alexa Fluor 594 (Thermo Fisher) at a dilution of 1:200 in PBS for 30 min at room temperature in the dark. After washing four times with 1×PBS, images were acquired using the EVOS Cell Imaging System (Thermo Fisher).

Structural analysis. PI3-E12 F_{ab} was produced by incubating each 10 mg of IgG with 10 μg of LysC (New England Biolabs) overnight at 37° C. followed by incubating with protein A for 1 hour at room temperature. The mixture was then centrifuged through a PVDF filter, concentrated in PBS with a 30 kDa Amicon Ultra size exclusion column, and purified further by size exclusion chromatography using Superdex 200 (GE Healthcare Life Sciences) in 5 mM Hepes and 150 mM NaCl. Crystals of PI3-E12 F_{ab} were obtained using a NT8 dispensing robot (Formulatrix), and screening was done using commercially available screens (Rigaku Wizard Precipitant Synergy block #2, Molecular Dimensions Proplex screen HT-96, Hampton Research Crystal Screen HT) by mixing 0.1 μL/0.1 μL (protein/reservoir) by the vapor diffusion method. Crystals used for diffraction data were grown in the following conditions in solution containing 0.2 M ammonium phosphate monobasic, 0.1 M Tris, pH 8.5, and 50% (+/−) 2-methyl-2,4-pentanediol. Crystals were cryoprotected in Parabar Oil (Hampton). Crystal diffracted to 2.1 Å (Table 3). Data was collected on the Fred Hutch X-ray home source and processed using HKL2000⁵⁵. The structure was solved by molecular replacement using Phaser in CCP4 (Collaborative Computational Project, Number 4) and PDB accession number 6MJZ for HPIV3 preF as a search model. Stewart-Jones, et al. Proc Natl Acad Sci USA 115, 12265-12270, (2018); Collaborative Computational Project, N. The CCP4 suite: programs for protein crystallography. Acta Crystallogr D Biol Crystallogr 50, 760-763, (1994). Iterating rounds of structure building and refinement was performed in COOT (Emsley & Cowtan, Acta Crystallogr D Biol Crystallogr 60, 2126-2132, (2004)) and Phenix (Adams, et al. Acta Crystallogr D Biol Crystallogr 66, 213-221, (2010)). Structural figures were made with Pymol (DeLano, Curr Opin Struct Biol 12, 14-20, (2002)) and Chimera (Pettersen, et al. J Comput Chem 25, 1605-1612, (2004)).

Complex of PI3-E12 F_{ab}+HPIV3 preF was formed by mixing both components at a 1:1 molar ratio and incubating overnight at 4° C. Complexes were purified by size exclusion chromatography using Superdex 200 in 5 mM Hepes and 150 mM NaCl at pH 7.4. Negative stain electron microscopy was performed as described in Kong, et al. Nat Struct Mol Biol 20, 796-803, (2013). Data were collected using a FEI Tecnai T12 electron microscope operating at 120 keV, with an electron dose of 30 e⁻ Å⁻² and a magnification of 52,000× that resulted in a pixel size of 1.6 Å at the specimen plane. Images were acquired with a Tietz TemCam-F416 CMOS camera using a nominal defocus range of 800 to 1,000 nm using the Leginon interface. Image processing was carried out using cisTEM. Grant, et al. Elife 7, doi:10.7554/eLife.35383 (2018). The final reconstruction was performed using 12,000 unbinned particles, refining for 20 iterations with C1 symmetry applied. PI3-E12 F_{ab} (this disclosure), HPIV3 (PDB ID 6MJZ (Stewart-Jones, et al. Proc Natl Acad Sci USA 115, 12265-12270, (2018)) and GNC4 (PDB ID 4DME) Oshaben, et al. Biochemistry 51, 9581-9591, (2012). Fitting was carried out using the Fit function in Chimera. Pettersen, et al. J Comput Chem 25, 1605-1612, (2004).

Animals and HPIV3 challenge. Cotton rat challenge experiments were performed with IACUC approval. Animals in groups of N=4-5 were infected intranasally with 100 μL of 10⁵ pfu HPIV3. Monoclonal antibody was either administered intramuscularly one day prior to infection or one day after infection. Cyclophosphamide (50 mg/kg) was administered intramuscularly at 21 days prior to infection and re-administered every three days for the duration of experiments involving immunosuppression. Nasal turbinates were removed for viral titration by plaque assay at day four post-infection. Lungs were removed for viral titration by plaque assay and histopathology at day four post-infection. Lung and nose homogenates were clarified by centrifugation in EMEM (Gibco). Confluent HEp-2 monolayers were inoculated in duplicate with diluted homogenates in 24-well plates. After incubating for two hours at 37° C., wells were overlaid with 0.75% methylcellulose. After four days, the cells were fixed and stained with 0.1% crystal violet for one hour, and plaques were counted to determine titers as pfu per gram of tissue. Histopathology was performed by inflating dissected lungs with 10% formalin, immersing in 10% formalin, embedding in paraffin, sectioning, and staining with hematoxylin and eosin. Slides were scored blind on a 0-4 severity scale. The scores are subsequently converted to a 0-100% histopathology scale as described. Boukhvalova, et al. Bone Marrow Transplant 51, 119-126, (2016); Porter, et al. J Virol 65, 103-111 (1991).

Statistical analysis. Statistical analysis was performed using GraphPad Prism 7. Pairwise statistical comparisons were performed using unpaired two-tailed t-test. P<0.05 was considered statistically significant. Data points from individual samples are displayed.

Prophetic Example 1. The Golden Syrian hamster or cotton rat will be used as a small animal model. For prophylaxis, 5 mg/kg 3×1 or control antibody will be given one day prior to infection of hamsters with HPIV1 & HPIV3 and viral titers will be measured in the nose and lungs four days after infection which is the time of peak viral replication in these animals. For therapy, 5 mg/kg 3×1 or control antibody will be given one day after infection. Groups of five animals will be included in each condition. Treatment with 3×1 will provide an anti-viral effect.

Prophetic Example 2. The Golden Syrian hamster or cotton rat will be used as a small animal model. For prophylaxis, 5 mg/kg MxR-01 or control antibody will be given one day prior to infection of hamsters with RSV or HMPV and viral titers will be measured in the nose and lungs four days after infection which is the time of peak viral replication in these animals. For therapy, 5 mg/kg MxR-01 or control antibody will be given one day after infection. Groups of five animals will be included in each condition. Treatment with MxR-01 will provide an anti-viral effect.

(vi) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding fragment (of, for example, a binding fragment) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding fragments may be classified as “high affinity” or “low affinity”. In particular embodiments, “high affinity” binding fragments refer to those binding fragments with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding fragments refer to those binding fragments with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding fragment may have “enhanced affinity,” which refers to a selected or engineered binding fragments with stronger binding to a cognate binding molecule than a wild type (or parent) binding fragment. For example, enhanced affinity may be due to a Ka (equilibrium association constant) for the cognate binding molecule that is higher than the reference binding fragment or due to a Kd (dissociation constant) for the cognate binding molecule that is less than that of the reference binding fragment, or due to an off-rate (Koff) for the cognate binding molecule that is less than that of the reference binding fragment. A variety of assays are known for detecting binding fragments that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in binding between a disclosed antibody and its viral epitope.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An antibody or binding fragment thereof comprising the complementarity determining regions (CDRs) of MxR-B11, PI3-E12, PI3-A3, PI3-B5, PI3-A10, PI3-A12, 3×1, or MxR-D10.

2. The antibody or binding fragment thereof of claim 1, wherein the CDRs are according to Kabat, Chothia, Martin, Contact, IMGT, AHo, or North numbering.

3. The antibody or binding fragment thereof of claim 1, comprising
a CDRH1 comprising GFPFSSYK (SEQ ID NO: 43); a CDRH2 comprising ISASGSYI (SEQ ID NO: 44); a CDRH3 comprising ARDGGRELSPFEK (SEQ ID NO: 45); a CDRL1 comprising NSNIGTGYD (SEQ ID NO: 46); a CDRL2 comprising DNN; and a CDRL3 comprising QSYDKSLGGWV (SEQ ID NO: 47);
a CDRH1 comprising GFTFSDHY (SEQ ID NO: 1); a CDRH2 comprising ISSSGSNT (SEQ ID NO: 2); a CDRH3 comprising ARAKWGTMGRGAPPTIYDH (SEQ ID NO: 3); a CDRL1 comprising QSLLQSNGNNY (SEQ ID NO: 4); a CDRL2 comprising LGS; and a CDRL3 comprising MQALQTPLT (SEQ ID NO: 5);
a CDRH1 comprising GFTFSNYW (SEQ ID NO: 8); a CDRH2 comprising VKEEGSEK (SEQ ID NO: 9); a CDRH3 comprising AGEVKSGWFGRYFDS (SEQ ID NO: 10); a CDRL1 comprising QSVGSW (SEQ ID NO: 11); a CDRL2 comprising KTS; and a CDRL3 comprising QQYSSFPYT (SEQ ID NO: 12);
a CDRH1 comprising GYNFTNYW (SEQ ID NO: 15); a CDRH2 comprising IYPADSDT (SEQ ID NO: 16); a CDRH3 comprising ARPSTRWFVPGGMDV (SEQ ID NO: 17); a CDRL1 comprising QSIGAW (SEQ ID NO: 18); a CDRL2 comprising KAS; and a CDRL3 comprising QQHSSYPST (SEQ ID NO: 19);
a CDRH1 comprising GFNFNNYG (SEQ ID NO: 22); a CDRH2 comprising VSFDGSNR (SEQ ID NO: 23); a CDRH3 comprising SKSKYSDFWSEI (SEQ ID NO: 24); a CDRL1 comprising QNVMRY (SEQ ID NO: 25); a CDRL2 comprising DAS; and a CDRL3 comprising QQRTNHRFS (SEQ ID NO: 26);
a CDRH1 comprising GDSVKSDDFY (SEQ ID NO: 29); a CDRH2 comprising IYYGGRT (SEQ ID NO: 30); a CDRH3 comprising VRVEGLLWFGELFDY (SEQ ID NO: 31); a CDRL1 comprising NSNIGNNF (SEQ ID NO: 32); a CDRL2 comprising KDY; and a CDRL3 comprising AAWQDGLSGPL (SEQ ID NO: 33);
a CDRH1 comprising GFTFSSFG (SEQ ID NO: 36); a CDRH2 comprising ISHSAGFL (SEQ ID NO: 37); a CDRH3 comprising AKRLAGLPDLEWLLYPNFLDH (SEQ ID NO: 38); a CDRL1 comprising ILRTYY (SEQ ID NO: 39); a CDRL2 comprising GKN; and a CDRL3 comprising SSRDRSGNHVL (SEQ ID NO: 40);
or
a CDRH1 comprising GFIFSNYD (SEQ ID NO: 50); a CDRH2 comprising ITGGSSFI (SEQ ID NO: 51); a CDRH3 comprising ARDGGRQLSPCEH (SEQ ID NO: 52); a CDRL1 comprising SSNIGAGYD (SEQ ID NO: 53); a CDRL2 comprising DNN; and a CDRL3 comprising QSYDRGLSGWA (SEQ ID NO: 54).

4. The antibody or binding fragment thereof of claim 1, comprising
a variable heavy chain having a sequence with at least 95% sequence identity to the sequence as set forth in SEQ ID NO: 178 and a variable light chain having a sequence with at least 95% sequence identity to the sequence as set forth in SEQ ID NO: 179;
a variable heavy chain having at least 95% sequence identity to the sequence as set forth in SEQ ID NO: 166 and a variable light chain having a sequence with at least 95% sequence identity to the sequence as set forth in SEQ ID NO: 167;
a variable heavy chain having a sequence with at least 95% sequence identity to the sequence as set forth in SEQ ID NO: 168 and a variable light chain having a sequence with at least 95% sequence identity to the sequence as set forth in SEQ ID NO: 169;
a variable heavy chain having a sequence with at least 95% sequence identity to the sequence as set forth in SEQ ID NO: 170 and a variable light chain having a sequence with at least 95% sequence identity to the sequence as set forth in SEQ ID NO: 171;
a variable heavy chain having a sequence with at least 95% sequence identity to the sequence as set forth in SEQ ID NO: 172 and a variable light chain having a sequence with at least 95% sequence identity to the sequence as set forth in SEQ ID NO: 173;
a variable heavy chain having a sequence with at least 95% sequence identity to the sequence as set forth in SEQ ID NO: 174 and a variable light chain having a sequence with at least 95% sequence identity to the sequence as set forth in SEQ ID NO: 175;
a variable heavy chain having a sequence with at least 95% sequence identity to the sequence as set forth in SEQ ID NO: 176 and a variable light chain having a sequence with at least 95% sequence identity to the sequence as set forth in SEQ ID NO: 177;
or
a variable heavy chain having a sequence with at least 95% sequence identity to the sequence as set forth in SEQ ID NO: 180 and a variable light chain having a sequence with at least 95% sequence identity to the sequence as set forth in SEQ ID NO: 181.

5. An antibody or binding fragment thereof comprising
a variable heavy chain having the sequence as set forth in SEQ ID NO: 178 and a variable light chain having the sequence as set forth in SEQ ID NO: 179;
a variable heavy chain having the sequence as set forth in SEQ ID NO: 166 and a variable light chain having the sequence as set forth in SEQ ID NO: 167;
a variable heavy chain having the sequence as set forth in SEQ ID NO: 168 and a variable light chain having the sequence as set forth in SEQ ID NO: 169;
a variable heavy chain having the sequence as set forth in SEQ ID NO: 170 and a variable light chain having the sequence as set forth in SEQ ID NO: 171;
a variable heavy chain having the sequence as set forth in SEQ ID NO: 172 and a variable light chain having the sequence as set forth in SEQ ID NO: 173;
a variable heavy chain having the sequence as set forth in SEQ ID NO: 174 and a variable light chain having the sequence as set forth in SEQ ID NO: 175;
a variable heavy chain having the sequence as set forth in SEQ ID NO: 176 and a variable light chain having the sequence as set forth in SEQ ID NO: 177;
or
a variable heavy chain having the sequence as set forth in SEQ ID NO: 180 and a variable light chain having the sequence as set forth in SEQ ID NO: 181

6. An antibody or binding fragment thereof encoded by a sequence having
at least 95% sequence identity to the sequence as set forth in SEQ ID NO: 48 and SEQ ID NO: 49;
at least 95% sequence identity to the sequence as set forth in SEQ ID NO: 6 and SEQ ID NO: 7;
at least 95% sequence identity to the sequence as set forth in SEQ ID NO: 13 and SEQ ID NO: 14;
at least 95% sequence identity to the sequence as set forth in SEQ ID NO: 27 and SEQ ID NO: 28;
at least 95% sequence identity to the sequence as set forth in SEQ ID NO: 34 and SEQ ID NO: 35;
at least 95% sequence identity to the sequence as set forth in SEQ ID NO: 41 and SEQ ID NO: 42;
or
at least 95% sequence identity to the sequence as set forth in SEQ ID NO: 55 and SEQ ID NO: 56.

7. An antibody or binding fragment thereof encoded by a sequence comprising the sequence as set forth in SEQ ID NO: 48 and SEQ ID NO: 49; SEQ ID NO: 6 and SEQ ID NO: 7; SEQ ID NO: 13 and SEQ ID NO: 14; SEQ ID NO: 27 and SEQ ID NO: 28; SEQ ID NO: 34 and SEQ ID NO:
35; SEQ ID NO: 41 and SEQ ID NO: 42; or SEQ ID NO: 55 and SEQ ID NO: 56.

8. The antibody of claim 1, wherein the antibody is an IgG antibody with M252Y, S254T, and T256E mutations in the Fc region.

9. A binding molecule comprising a binding fragment of at least 2 antibodies of claim 1.

10. The binding molecule of claim 9, wherein the binding fragments of at least 2 antibodies of claim 1 comprise Fab or single chain variable fragments (scFv).

11. The binding molecule of claim 9, wherein the binding fragments comprise Fab linked through a knobs-into-holes linkage of heavy chains.

12. The binding molecule of claim 9, wherein the binding fragments comprise an ScFv with one binding fragment linked to the constant region of an antibody having a different binding fragment.

13. The binding molecule of claim 12, wherein the linker comprises a Gly-Ser linker.

14. The binding molecule of claim 9, wherein the binding fragments of at least 2 antibodies of claim 1 comprise scFv.

15. The binding molecule of claim 9, comprising
a binding fragment of a PI3 antibody and a binding fragment of an MxR antibody;
a binding fragment of a PI3 antibody and a binding fragment of the 3×1 antibody; or
a binding fragment of the 3×1 antibody and a binding fragment of an MxR antibody.

16. The binding molecule of claim 9, comprising a binding fragment of a PI3 antibody, a binding fragment of the 3×1 antibody, and a binding fragment of an MxR antibody.

17. The binding molecule of claim 9, wherein the binding fragments comprise Fab and/or scFv.

18. The binding molecule of claim 9, comprising the MxR-B11 Fab and the 3×1 Fab; a MxR-B11 scFv and the 3×1 Fab; the MxR-B11 Fab and a 3×1 scFv; or a MxR-B11 scFv and a 3×1 scFv.

19. The binding molecule of claim 9, comprising the MxR-D10 Fab and the 3×1 Fab; a MxR-D10 scFv and the 3×1 Fab; the MxR-D10 Fab and a 3×1 scFv; or a MxR-D10 scFv and a 3×1 scFv.

20. A binding molecule comprising a binding fragment of claim 1 and a binding fragment of an antibody that binds a secondary virus.

21. The binding molecule of claim 20, wherein the secondary virus is selected from an adenovirus, a boca virus, a coronavirus, an enterovirus, an influenza virus, a metapneumovirus, a parainfluenza virus, and/or a rhinovirus.

22. The binding molecule of claim 20, wherein the secondary virus is selected from human adenovirus, human boca virus (HBoV), and/or human coronavirus (HCoV).

23. The binding molecule of claim 20, wherein the secondary virus is a coronavirus selected from SARS-CoV, MERS-CoV, coronavirus 229E, coronavirus OC43, coronavirus NL63, coronavirus HKU1, coronavirus NL, and/or coronavirus NH.

24. The binding molecule of claim 20, wherein the secondary virus is selected from influenza group A, influenza group B, human parainfluenza virus 2 (HPIV2), HPIV4, human rhinovirus (HRV)A, HRVB, and/or HRVC.

25. The binding molecule of claim 20, wherein the binding fragment comprises the CDRs of antibody 8C4, 5Hx-1, 5Hx-2, 5Hx-3, 5Hx-4, 5Hx-5, 5.100K-1, 5PB-1, 5Fb-1, 1E11, EPR23305-44 47D11, CR3022, CDC2-A2, G2, 5F9, FIB-H1, JC57-13, 32D6, CH65, CR9114, MAb 22/1, MAb70/l, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, PEG-1, CR8033, CR8071, 113/2, 124/4, 128/2, 134/1, 146/1, 152/2, 160/1, 162/1, 195/3, 206/2, 238/4, 280/2, PAR2 (boca231/9F), or TCN-711.

26. A binding molecule comprising a binding fragment of claim 1 and a binding fragment that activates an immune cell.

27. The binding molecule of claim 26, wherein the binding fragment that activates an immune cell binds CD3, CD28, or 4-1BB.

28. The binding molecule of claim 26, wherein the binding fragment that activates an immune cell comprises the CDRs of OKT3 or TGN1412.

29. A composition comprising an antibody or binding fragment of claim 1 and a pharmaceutically-acceptable carrier.

30. The composition of claim 29, wherein the pharmaceutically-acceptable carrier comprises an aqueous solution.

31. The composition of claim 29, wherein the pharmaceutically-acceptable carrier comprises physiological saline.

32. The composition of claim 29, wherein the pharmaceutically-acceptable carrier comprises sodium chloride, potassium chloride and calcium chloride.

33. The composition of claim 32, wherein the pharmaceutically-acceptable carrier further comprises sodium bicarbonate or sodium lactate.

34. The composition of claim 29, wherein the pharmaceutically-acceptable carrier comprises inorganic salts.

35. The composition of claim 34, wherein the pharmaceutically-acceptable carrier further comprises a carbohydrate.

36. The composition of claim 29, wherein the pharmaceutically-acceptable carrier comprises an antioxidant, a buffering agent, a chelating agent, an isotonic agent, a preservative, and/or a stabilizer.

37. The composition of claim 29, wherein the pharmaceutically-acceptable carrier comprises a release modifier.

38. The composition of claim 29, wherein the composition comprises a therapeutically-effective amount of the antibody or binding fragment for administration to a subject.

39. A method of providing an anti-viral effect in a subject in need thereof comprising administering a therapeutically effective amount of the composition of claim 29 to the subject thereby providing the anti-viral effect.

40. The method of claim 39, wherein the anti-viral effect comprises an anti-HPIV3 effect, an anti-HPIV1 effect, an anti-respiratory syncytial virus (RSV) effect, and/or an anti-human metapneumovirus (HMPV) effect.

41. The method of claim 39, wherein the anti-viral effect comprises an anti-HPIV3 effect and an anti-HPIV1 effect.

42. The method of claim 39, wherein the anti-viral effect comprises an anti-RSV effect and an anti-HMPV effect.

43. The method of claim 39, wherein the anti-viral effect comprises an anti-HPIV effect, an anti-RSV effect and an anti-HMPV effect.

44. The method of claim 39, wherein the subject in need thereof is a hematopoietic stem cell transplant (HCT) recipient, a lung transplant recipient, a premature infant, a person over 65 years of age, a homeless person, or a person with lung disease.

45. The method of claim 39, wherein the subject has chronic obstructive pulmonary disease (COPD).

46. The method of claim 39, wherein the anti-viral effect comprises an anti-viral effect against a secondary virus.

47. The method of claim 46, wherein the secondary virus is selected from an adenovirus, a boca virus, a coronavirus, an enterovirus, an influenza virus, a metapneumovirus, a parainfluenza virus, and/or a rhinovirus.

48. The method of claim 46, wherein the secondary virus is selected from human adenovirus, human boca virus (HBoV), and/or human coronavirus (HCoV).

49. The method of claim 46, wherein the secondary virus is a coronavirus selected from SARS-CoV, MERS-CoV, coronavirus 229E, coronavirus OC43, coronavirus NL63, coronavirus HKU1, coronavirus NL, and/or coronavirus NH.

50. The method of claim 46, wherein the secondary virus is selected from influenza group A, influenza group B, human parainfluenza virus 2 (HPIV2), HPIV4, human rhinovirus (HRV)A, HRVB, and/or HRVC.


SEQUENCE LISTING (194 sequences)

SEQ ID NO: 1 - protein, 8 aa, Artificial Sequence
  mAb PI3-E12 CDRH1
GFTFSDHY

SEQ ID NO: 2 - protein, 8 aa, Artificial Sequence
  mAb PI3-E12 CDRH2
ISSSGSNT

SEQ ID NO: 3 - protein, 19 aa, Artificial Sequence
  mAb PI3-E12 CDRH3
ARAKWGTMGRGAPPTIYDH

SEQ ID NO: 4 - protein, 11 aa, Artificial Sequence
  mAb PI3-E12 CDRL1
QSLLQSNGNNY

SEQ ID NO: 5 - protein, 9 aa, Artificial Sequence
  mAb PI3-E12 CDRL3
MQALQTPLT

SEQ ID NO: 6 - DNA, 378 bp, Artificial Sequence
  PI3-E12 variable heavy chain
caggtgcagctgttggagtctgggggaaagttggtcaagcctggagggtccctgagactctcctgtgcagcctctggattcaccttcagtgaccactacatgatctggatccgccaggctcccgggaaggggctggagtggatttcatacataagtagtagtggtagtaacacaatctacgcagactctttgatgggccgattcaccatctccagagacaacgccaagaactctctgtatctacaaatgaacagcctgaggaccgaggacacggccgtttattactgtgcgagagcaaagtggggtactatgggtcggggagcacccccgacaatttatgaccactggggccagggaaccctggtcaccgtctcctca

SEQ ID NO: 7 - DNA, 337 bp, Artificial Sequence
  PI3-E12 variable kappa light chain
gatattgtgatgactcagtctccactctccctgcccgtcacccctggagagccggcctccatctcctgtaggtctagtcagagcctcctgcaaagtaatggaaacaattatttggagtggtacctgcagaagccagggcagtctccacaactcctgatctatttgggttccaatcgggcctccggggtccctgacaggttcagtggcagtggatcaggcacagattttacactgaagatcagcagagtggaggctgaggatgctggggtttattactgcatgcaagctctacaaactccgctcactttcggcggagggaccaaggtggagatcaaac

SEQ ID NO: 8 - protein, 8 aa, Artificial Sequence
  mAb PI3-A3 CDRH1
GFTFSNYW

SEQ ID NO: 9 - protein, 8 aa, Artificial Sequence
  mAb PI3-A3 CDRH2
VKEEGSEK

SEQ ID NO: 10 - protein, 15 aa, Artificial Sequence
  mAb PI3-A3 CDRH3
AGEVKSGWFGRYFDS

SEQ ID NO: 11 - protein, 6 aa, Artificial Sequence
  mAb PI3-A3 CDRL1
QSVGSW

SEQ ID NO: 12 - protein, 9 aa, Artificial Sequence
  mAb PI3-A3 CDRL3
QQYSSFPYT

SEQ ID NO: 13 - DNA, 367 bp, Artificial Sequence
  PI3-A3 antibody variable heavy chain
gaggtgcagctggtggagtctgggggaggcttggtccagcctggggggtccctgagactctcctgtacagcctctggattcacctttagcaattattggatgagctgggtccgccaggctccagggaaggggctggagtgggtggccaatgtgaaggaagaaggaagtgagaaacactatgtagactctgtgaagggccgattcaccatctccagagacaacgccaagaactcagtgtatctgcagatgagcagcctgagagccgaggacacggctgtctattactgtgcgggagaggttaagagtggctggttcggtcggtactttgactcctggggccagggaaccctggtcaccgtctcctcag

SEQ ID NO: 14 - DNA, 322 bp, Artificial Sequence
  PI3-A3 antibody variable kappa light chain
gacatccagatgacccagtctccttccaccctgtctgcatctgttggagacagagtcaccatcaattgccgggccagtcagagtgttggtagctggttggcctggtatcagcagaaaccagggaaagcccctaagctcctgatgtataagacatctactttacaaagaggggtcccatcaaggttcagcggcagtggatctgggacagaattcactctcaccatcagcagcctgcagcctgatgattttgcagcttattactgccaacagtatagtagttttccgtacacttttggccaggggaccaagctggagatcaaac

SEQ ID NO: 15 - protein, 8 aa, Artificial Sequence
  mAb PI3-B5 CDRH1
GYNFTNYW

SEQ ID NO: 16 - protein, 8 aa, Artificial Sequence
  mAb PI3-B5 CDRH2
IYPADSDT

SEQ ID NO: 17 - protein, 15 aa, Artificial Sequence
  mAb PI3-B5 CDRH3
ARPSTRWFVPGGMDV

SEQ ID NO: 18 - protein, 6 aa, Artificial Sequence
  mAb PI3-B5 CDRL1
QSIGAW

SEQ ID NO: 19 - protein, 9 aa, Artificial Sequence
  mAb PI3-B5 CDRL3
QQHSSYPST

SEQ ID NO: 20 - DNA, 366 bp, Artificial Sequence
  PI3-B5 variable heavy chain
gaggtgcagctggtgcagtctggagcagaggtgaaaaagcccggggagtctctgaggatctcctgtaagggttctggatacaactttaccaactactggatcgcctgggtgcgccagatgcccgggaaaggcctggagtggatggggatcatctatcctgctgactcggataccagatacagcccgtccttccaaggccaagtcaccatctcagccgacaagtccatcaccaccgcctacctgcagtggagcagcctgaaggcctcggacaccgccatatactactgtgcgagaccgagtactaggtggttcgtccctggcggtatggacgtctggggccaaggcaccacggtcatcgtctcctca

SEQ ID NO: 21 - DNA, 322 bp, Artificial Sequence
  PI3-B5 variable kappa light chain
gacatccagatgacccagtctccttccaccctgtctgcttctgtaggagacagagtcaccatttcttgccgggccactcagagtattggtgcctggttggcctggtatcagcagaaaccaggggaaccccctaagctcctgatctataaggcgtctactttagagagtggggtcccatcaaggttcagcggcagtggatctgggacagaattcactctcaccatcagcagcctgcagcctgatgattctgcaacttattactgccaacagcatagtagttatccttctactttcggcggagggaccaaggtggagatcaaac

SEQ ID NO: 22 - protein, 8 aa, Artificial Sequence
  mAb PI3-A10 CDRH1
GFNFNNYG

SEQ ID NO: 23 - protein, 8 aa, Artificial Sequence
  mAb PI3-A10 CDRH2
VSFDGSNR

SEQ ID NO: 24 - protein, 12 aa, Artificial Sequence
  mAb PI3-A10 CDRH3
SKSKYSDFWSEI

SEQ ID NO: 25 - protein, 6 aa, Artificial Sequence
  mAb PI3-A10 CDRL1
QNVMRY

SEQ ID NO: 26 - protein, 9 aa, Artificial Sequence
  mAb PI3-A10 CDRL3
QQRTNHRFS

SEQ ID NO: 27 - DNA, 358 bp, Artificial Sequence
  PI3-A10 variable heavy chain
caggtgcagctggtggagtctgggggaggcgtggtccggcctgggaggtccctgagactctcctgtgtagcctctggattcaacttcaataactatgggctgcagtggatccgccaggctccaggcaaggggctggagtgggtggcaggtgtctcgtttgatgggagtaatagatattatgcagactccgtgaagggccgagtcaccatatccagagacgattccaagaacaccctgtatctagaaatgaacagcctgagagctgaggacacaggaatatattactgttcgaagtccaagtactccgacttttggagcgaaatatggggccagggaaccctggtcaccgtctcctcag

SEQ ID NO: 28 - DNA, 322 bp, Artificial Sequence
  PI3-A10 variable kappa light chain
gaaattgtgttgacacagtctccagccaccctgtctttgtctccaggggaaagagccaccctctcctgcagggccagtcagaatgttatgaggtacttagcctggtaccaacagagacctggccaggctcccagactcctcttctatgatgcatccagccgggccactggcatcccagcccggttcactgccagtgggtctgggacagacttcactctcaccatcagcggcctcgagcctggagattttgcagtttattactgtcagcagcgtaccaaccatagattctctttcggccctgggaccaaggtggatatcaaac

SEQ ID NO: 29 - protein, 10 aa, Artificial Sequence
  mAb PI3-A12 CDRH1
GDSVKSDDFY

SEQ ID NO: 30 - protein, 7 aa, Artificial Sequence
  mAb PI3-A12 CDRH2
IYYGGRT

SEQ ID NO: 31 - protein, 15 aa, Artificial Sequence
  mAb PI3-A12 CDRH3
VRVEGLLWFGELFDY

SEQ ID NO: 32 - protein, 8 aa, Artificial Sequence
  mAb PI3-A12 CDRL1
NSNIGNNF

SEQ ID NO: 33 - protein, 11 aa, Artificial Sequence
  mAb PI3-A12 CDRL3
AAWQDGLSGPL

SEQ ID NO: 34 - DNA, 370 bp, Artificial Sequence
  PI3-A12 variable heavy chain
caggtgcagctgcaggagtcgggcccaggactggtgaagccttcagagaccctgtccctcacttgcactgtctctggtgactccgtcaagagtgatgatttctactggagttggatccggcagcccccagggaagggcctggagtggattggcttcatctattacggtggcagaacttactacaacccgtccctcagtggtcgaggaaccatttcagtggacacgtccaagaaccacttcttcctggagctgacctctgtgactgccgcagacacggccgtatactactgtgtcagggtcgaaggattactgtggttcggggagttattcgactactggggccagggaaccctggtcaccgtctcctcag

SEQ ID NO: 35 - DNA, 331 bp, Artificial Sequence
  PI3-A12 variable lambda light chain
cagtctgtgctgacgcagccaccctcagcgtctgggacccccgggcagagggtcaccatctcttgttctggaagcaactccaacatcggaaataattttgtctactggtaccaacaagtcccaggatcggcccccaaagtcgtcatttacaaggattatcagcgcccctcaggggtccctgaccgattctctgcctccaagtctggcacctcagcctccctgaccatcagcgggctccggtccgacgatgaggcccattattactgtgcagcatggcaggacggtctgagtgggccgttatttggcggagggaccaagctgaccgtcctag

SEQ ID NO: 36 - protein, 8 aa, Artificial Sequence
  mAb 3x1 CDRH1
GFTFSSFG

SEQ ID NO: 37 - protein, 8 aa, Artificial Sequence
  mAb 3x1 CDRH2
ISHSAGFL

SEQ ID NO: 38 - protein, 21 aa, Artificial Sequence
  mAb 3x1 CDRH3
AKRLAGLPDLEWLLYPNFLDH

SEQ ID NO: 39 - protein, 6 aa, Artificial Sequence
  mAb 3x1 CDRL1
ILRTYY

SEQ ID NO: 40 - protein, 11 aa, Artificial Sequence
  mAb 3x1 CDRL3
SSRDRSGNHVL

SEQ ID NO: 41 - DNA, 385 bp, Artificial Sequence
  3x1 xnAb variable heavy chain
gaggtgcagctgttggagtctgggggaggcttggtacagcctggtgggtccctgagactgtcttgtgcggcctcaggattcacctttagcagctttggcatgagctgggtccgccaatctccaggaaaggggctggagtgggtcgcagatataagccatagtgctggcttcttaaactacgcagactccgtgaagggccggttcaccgtctccagagacaattctaagagcacgctgcatctccaaatgaagagcctgagagccgaggacacggccgtatattactgtgcgaaaagacttgccggattaccagatttggagtggttactttaccctaacttcttagaccactggggccagggaaccctggtcaccgtctcctcag

SEQ ID NO: 42 - DNA, 325 bp, Artificial Sequence
  3x1 xnAb variable lambda light chain
tcttctgagctgactcaggaccctgctgtgtctgtggccttgggacagacagtcaggatcacatgccaaggagacatactgagaacctattatgtaagctggtaccagcagaaaccaggacaggccccgctacttgtcatctatggtaaaaacaaccgaccctcagtgatcccagaccgattctctggctccacctcaggagacacagcttccttgaccatcactggggctcaggcggaagatgaggctgagtattattgtagctctcgggacaggagtggaaaccatgtgctattcggcggagggaccaagctgaccgtcctag

SEQ ID NO: 43 - protein, 8 aa, Artificial Sequence
  mAb MxR-B11 CDRH1
GFPFSSYK

SEQ ID NO: 44 - protein, 8 aa, Artificial Sequence
  mAb MxR-B11 CDRH2
ISASGSYI

SEQ ID NO: 45 - protein, 13 aa, Artificial Sequence
  mAb MxR-B11 CDRH3
ARDGGRELSPFEK

SEQ ID NO: 46 - protein, 9 aa, Artificial Sequence
  mAb MxR-B11 CDRL1
NSNIGTGYD

SEQ ID NO: 47 - protein, 11 aa, Artificial Sequence
  mAb MxR-B11 CDRL3
QSYDKSLGGWV

SEQ ID NO: 48 - DNA, 361 bp, Artificial Sequence
  MxR-B11 variable heavy chain
gaggtgcaggtggtggagtctgggggaggcctggtcaagcctggggggtccctgagactctcctgtgcagcctctggattccccttcagttcttataagatggactgggtccgccaggctccagggaaggggctggagtgggtctcgtccatcagtgctagtggaagttacataaactatgcagactcagtgaagggccgattcaccatctccagagacaacgccaagaactcactgtatctgcaaatgaaaagcctgagagccgacgacacggctgtatatttttgtgcgagagacggcggaagagaactgagcccctttgaaaagtggggccagggaatcctggtcaccgtctcctcag

SEQ ID NO: 49 - DNA, 334 bp, Artificial Sequence
  MxR-B11 variable light chain
cagtctgtgctgacgcagccgccctcagtgtctggggccccaggacagagggtcaccatctcctgcactgggaccaactccaacatcgggacaggttatgatgtacactggtaccagcagcttccgggaacagcccccaaagtcgtcctctttgataacaacaatcggccctcaggggtccctgaccgattctctggctccaagtctggcacttcagccgccctggccatcactggcctccaggctgaggatgaggctgtttattactgccagtcctatgacaagagcctgggtggttgggtgttcggcggagggaccaagctgaccgtcctag

SEQ ID NO: 50 - protein, 8 aa, Artificial Sequence
  mAb MxR-D10 CDRH1
GFIFSNYD

SEQ ID NO: 51 - protein, 8 aa, Artificial Sequence
  mAb MxR-D10 CDRH2
ITGGSSFI

SEQ ID NO: 52 - protein, 13 aa, Artificial Sequence
  mAb MxR-D10 CDRH3
ARDGGRQLSPCEH

SEQ ID NO: 53 - protein, 9 aa, Artificial Sequence
  mAb MxR-D10 CDRL1
SSNIGAGYD

SEQ ID NO: 54 - protein, 11 aa, Artificial Sequence
  mAb MxR-D10 CDRL3
QSYDRGLSGWA

SEQ ID NO: 55 - DNA, 361 bp, Artificial Sequence
  MxR-D10 variable heavy chain
gaggtgcaggtggtggagtcggggggaggcctggtcaagcctggggggtccctgagactctcctgtacagcctctggattcatattcagtaattatgacatgaactgggtccgccaggctccagggaagggcctggagtgggtcgcctccattactggtggtagtagtttcataaattacgcagactcagtgaagggccgattcaccatctccagagacaacgccaagaagtcactgtatctgcaaatgaacagcctcagagccgaggacacggctgtctattactgtgcgagagatggcgggagacagttgagtccgtgtgaacattggggccagggaaccctggtcaccgtctcctcag

SEQ ID NO: 56 - DNA, 334 bp, Artificial Sequence
  MxR-D10 variable light chain
cagtctgtgctgacgcagccgccctcagtgtctgggtccccagggcagagggtcaccatctcctgcactgggggcagctccaacatcggggcaggttatgatgtacactggtaccaacaacttccaggatcagcccccaaactcctcatgtatgatagtaataatcgaccctcaggggtccctgaccgattctctggctccaagtctggcacctcagcctccctggccatcactgggctccaggctgaggatgaggctgattattactgccagtcctatgacaggggcctgagtgggtgggcgttcggcggagggaccaagctgaccgtcctgg

SEQ ID NO: 57 - protein, 8 aa, Artificial Sequence
  anti-MERS-CoV CDRH1
EFTFNTYG

SEQ ID NO: 58 - protein, 8 aa, Artificial Sequence
  anti-MERS-CoV CDRH2
ISYDGTKK

SEQ ID NO: 59 - protein, 11 aa, Artificial Sequence
  anti-MERS-CoV CDRH3
ARSGDSDAFDI

SEQ ID NO: 60 - protein, 6 aa, Artificial Sequence
  anti-MERS-CoV CDRL1
ELGDKF

SEQ ID NO: 61 - protein, 9 aa, Artificial Sequence
  anti-MERS-CoV CDRL3
QAWDSNSYV

SEQ ID NO: 62 - protein, 8 aa, Artificial Sequence
  anti-MERS-CoV CDRH1
GGTFGSYA

SEQ ID NO: 63 - protein, 8 aa, Artificial Sequence
  anti-MERS-CoV CDRH2
IDAANGNT

SEQ ID NO: 64 - protein, 13 aa, Artificial Sequence
  anti-MERS-CoV CDRH3
ARDRWMTTRAFDI

SEQ ID NO: 65 - protein, 8 aa, Artificial Sequence
  anti-MERS-CoV CDRL1
SSNIGSNY

SEQ ID NO: 66 - protein, 11 aa, Artificial Sequence
  anti-MERS-CoV CDRL3
AAWDDSLRGPV

SEQ ID NO: 67 - protein, 10 aa, Artificial Sequence
  OKT3 CDRL1
SASSSVSYMN

SEQ ID NO: 68 - protein, 11 aa, Artificial Sequence
  OKT3 CDRL2
RWIYDTSKLAS

SEQ ID NO: 69 - protein, 9 aa, Artificial Sequence
  OKT3 CDRL3
QQWSSNPFT

SEQ ID NO: 70 - protein, 13 aa, Artificial Sequence
  OKT3 CDRH1
KASGYTFTRYTMH

SEQ ID NO: 71 - protein, 16 aa, Artificial Sequence
  OKT3 CDRH2
INPSRGYTNYNQKFKD

SEQ ID NO: 72 - protein, 10 aa, Artificial Sequence
  OKT3 CDRH3
YYDDHYCLDY

SEQ ID NO: 73 - protein, 242 aa, Artificial Sequence
  anti-CD3 scFv
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 74 - protein, 11 aa, Artificial Sequence
  anti-CD3 CDRL1
QSLVHNNGNTY

SEQ ID NO: 75 - protein, 9 aa, Artificial Sequence
  anti-CD3 CDRL3
GQGTQYPFT

SEQ ID NO: 76 - protein, 8 aa, Artificial Sequence
  anti-CD3 CDRH1
GFTFTKAW

SEQ ID NO: 77 - protein, 10 aa, Artificial Sequence
  anti-CD3 CDRH2
IKDKSNSYAT

SEQ ID NO: 78 - protein, 12 aa, Artificial Sequence
  anti-CD3 CDRH3
RGVYYALSPFDY

SEQ ID NO: 79 - protein, 11 aa, Artificial Sequence
  anti-CD3 CDRL1
QSLVHDNGNTY

SEQ ID NO: 80 - protein, 8 aa, Artificial Sequence
  anti-CD3 CDRH1
GFTFSNAW

SEQ ID NO: 81 - protein, 10 aa, Artificial Sequence
  anti-CD3 CDRH2
IKARSNNYAT

SEQ ID NO: 82 - protein, 12 aa, Artificial Sequence
  anti-CD3 CDRH3
RGTYYASKPFDY

SEQ ID NO: 83 - protein, 11 aa, Artificial Sequence
  anti-CD3 CDRL1
QSLEHNNGNTY

SEQ ID NO: 84 - protein, 10 aa, Artificial Sequence
  anti-CD3 CDRH2
IKDKSNNYAT

SEQ ID NO: 85 - protein, 13 aa, Artificial Sequence
  anti-CD3 CDRH3
RYVHYGIGYAMDA

SEQ ID NO: 86 - protein, 11 aa, Artificial Sequence
  anti-CD3 CDRL1
QSLVHTNGNTY

SEQ ID NO: 87 - protein, 9 aa, Artificial Sequence
  anti-CD3 CDRL3
GQGTHYPFT

SEQ ID NO: 88 - protein, 8 aa, Artificial Sequence
  anti-CD3 CDRH1
GFTFTNAW

SEQ ID NO: 89 - protein, 9 aa, Artificial Sequence
  anti-CD3 CDRH2
KDKSNNYAT

SEQ ID NO: 90 - protein, 13 aa, Artificial Sequence
  anti-CD3 CDRH3
RYVHYRFAYALDA

SEQ ID NO: 91 - protein, 120 aa, Artificial Sequence
  variable heavy chain of TGN1412
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 92 - protein, 107 aa, Artificial Sequence
  variable light chain of TGN1412
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 93 - protein, 11 aa, Artificial Sequence
  anti-CD28 CDRL1
HASQNIYVWLN

SEQ ID NO: 94 - protein, 7 aa, Artificial Sequence
  anti-CD28 CDRL2
KASNLHT

SEQ ID NO: 95 - protein, 9 aa, Artificial Sequence
  anti-CD28 CDRL3
QQGQTYPYT

SEQ ID NO: 96 - protein, 10 aa, Artificial Sequence
  anti-CD28 CDRH1
GYTFTSYYIH

SEQ ID NO: 97 - protein, 14 aa, Artificial Sequence
  anti-CD28 CDRH2
CIYPGNVNTNYNEK

SEQ ID NO: 98 - protein, 11 aa, Artificial Sequence
  anti-CD28 CDRH3
SHYGLDWNFDV

SEQ ID NO: 99 - protein, 5 aa, Artificial Sequence
  anti-CD28 CDRH1
SYYIH

SEQ ID NO: 100 - protein, 17 aa, Artificial Sequence
  anti-CD28 CDRH2
CIYPGNVNTNYNEKFKD

SEQ ID NO: 101 - protein, 7 aa, Artificial Sequence
  anti-4-1BB CDRL1
RASQSVS

SEQ ID NO: 102 - protein, 6 aa, Artificial Sequence
  anti-4-1BB CDRL2
ASNRAT

SEQ ID NO: 103 - protein, 10 aa, Artificial Sequence
  anti-4-1BB CDRL3
QRSNWPPALT

SEQ ID NO: 104 - protein, 4 aa, Artificial Sequence
  anti-4-1BB CDRH1
YYWS

SEQ ID NO: 105 - protein, 12 aa, Artificial Sequence
  anti-4-1BB CDRH3
YGPGNYDWYFDL

SEQ ID NO: 106 - protein, 11 aa, Artificial Sequence
  anti-4-1BB CDRL1
SGDNIGDQYAH

SEQ ID NO: 107 - protein, 7 aa, Artificial Sequence
  anti-4-1BB CDRL2
QDKNRPS

SEQ ID NO: 108 - protein, 11 aa, Artificial Sequence
  anti-4-1BB CDRL3
ATYTGFGSLAV

SEQ ID NO: 109 - protein, 10 aa, Artificial Sequence
  anti-4-1BB CDRH1
GYSFSTYWIS

SEQ ID NO: 110 - protein, 14 aa, Artificial Sequence
  anti-4-1BB CDRH2
KIYPGDSYTNYSPS

SEQ ID NO: 111 - protein, 7 aa, Artificial Sequence
  anti-4-1BB CDRH3
GYGIFDY

SEQ ID NO: 112 - protein, 11 aa, Artificial Sequence
  anti-CD8 CDRL1
RTSRSISQYLA

SEQ ID NO: 113 - protein, 7 aa, Artificial Sequence
  anti-CD8 CDRL2
SGSTLQS

SEQ ID NO: 114 - protein, 9 aa, Artificial Sequence
  anti-CD8 CDRL3
QQHNENPLT

SEQ ID NO: 115 - protein, 6 aa, Artificial Sequence
  anti-CD8 CDRH1
GFNIKD

SEQ ID NO: 116 - protein, 9 aa, Artificial Sequence
  anti-CD8 CDRH2
RIDPANDNT

SEQ ID NO: 117 - protein, 9 aa, Artificial Sequence
  anti-CD8 CDRH3
GYGYYVFDH

SEQ ID NO: 118 - protein, 109 aa, Artificial Sequence
  anti-KIR2DL1 and KIR2DL2/3 variable light chain
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 119 - protein, 123 aa, Artificial Sequence
  anti-KIR2DL1 and KIR2DL2/3 variable heavy chain
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 120 - skipped

SEQ ID NO: 121 - protein, 5 aa, Artificial Sequence
  GlySer linker
  MISC_FEATURE (1)..(5): (GlyGlyGlyGlySer) is repeated n times wherein n is an integer of 1, 2, 3, 4, 5, 6, 7, 8, 9 or 10
GGGGS

SEQ ID NO: 122 - protein, 9 aa, Artificial Sequence
  GlySer linker
  MISC_FEATURE (1)..(4): (GlyGlyGlySer) is repeated n times wherein n is an integer of 1, 2, 3, 4, 5, 6, 7, 8, 9 or 10
  MISC_FEATURE (5)..(9): (GlyGlyGlyGlySer) is repeated n times wherein n is an integer of 1, 2, 3, 4, 5, 6, 7, 8, 9 or 10
GGGSGGGGS

SEQ ID NO: 123 - protein, 7 aa, Artificial Sequence
  GlySer linker
  MISC_FEATURE (1)..(4): (GlyGlyGlySer) is repeated n times wherein n is an integer of 1, 2, 3, 4, 5, 6, 7, 8, 9 or 10
  MISC_FEATURE (5)..(7): (GlyGlySer) is repeated n times wherein n is an integer of 1, 2, 3, 4, 5, 6, 7, 8, 9 or 10
GGGSGGS

SEQ ID NO: 124 - protein, 9 aa, Artificial Sequence
  GlySer linker
  MISC_FEATURE (1)..(4): (GlyGlyGlySer) is repeated n times wherein n is an integer of 1, 2, 3, 4, 5, 6, 7, 8, 9 or 10
GGGSGGGGS

SEQ ID NO: 125 - protein, 20 aa, Artificial Sequence
  GlySer linker
GGGGSGGGGSGGGGSGGGGS

SEQ ID NO: 126 - protein, 15 aa, Artificial Sequence
  GlySer linker
GGGGSGGGGSGGGGS

SEQ ID NO: 127 - protein, 10 aa, Artificial Sequence
  GlySer linker
GGGGSGGGGS

SEQ ID NO: 128 - protein, 5 aa, Artificial Sequence
  GlySer linker
GGGGS

SEQ ID NO: 129 - protein, 8 aa, Artificial Sequence
  GlySer linker
GGGSGGGS

SEQ ID NO: 130 - protein, 4 aa, Artificial Sequence
  GlySer linker
GGGS

SEQ ID NO: 131 - protein, 6 aa, Artificial Sequence
  GlySer linker
GGSGGS

SEQ ID NO: 132 - protein, 11 aa, Artificial Sequence
  GlySer linker
GGSGGGSGGSG

SEQ ID NO: 133 - protein, 10 aa, Artificial Sequence
  GlySer linker
GGSGGGSGSG

SEQ ID NO: 134 - protein, 8 aa, Artificial Sequence
  GlySer linker
GGSGGGSG

SEQ ID NO: 135 - protein, 8 aa, Artificial Sequence
  anti-MERS-CoV CDRH1
GGTFSSYA

SEQ ID NO: 136 - protein, 8 aa, Artificial Sequence
  anti-MERS-CoV CDRH2
IIPIFGKA

SEQ ID NO: 137 - protein, 16 aa, Artificial Sequence
  anti-MERS-CoV CDRH3
ARDQGISANFKDAFDI

SEQ ID NO: 138 - protein, 6 aa, Artificial Sequence
  anti-MERS-CoV CDRL1
ESVGSN

SEQ ID NO: 139 - protein, 9 aa, Artificial Sequence
  anti-MERS-CoV CDRL3
QQYNNWPLT

SEQ ID NO: 140 - skipped

SEQ ID NO: 141 - protein, 8 aa, Artificial Sequence
  anti-MERS-CoV CDRH2
IIPIFGTA

SEQ ID NO: 142 - protein, 18 aa, Artificial Sequence
  anti-MERS-CoV CDRH3
ARVGYCSSTSCHIGAFDI

SEQ ID NO: 143 - protein, 6 aa, Artificial Sequence
  anti-MERS-CoV CDRL1
QSVSSS

SEQ ID NO: 144 - protein, 9 aa, Artificial Sequence
  anti-MERS-CoV CDRL3
QQYSSSPYT

SEQ ID NO: 145-146 - skipped (2 entries)

SEQ ID NO: 147 - protein, 18 aa, Artificial Sequence
  anti-MERS-CoV CDRH3
ARASYCSTTSCASGAFDI

SEQ ID NO: 148 - protein, 11 aa, Artificial Sequence
  anti-MERS-CoV CDRL1
QSVLYSSNNNY

SEQ ID NO: 149 - protein, 9 aa, Artificial Sequence
  anti-MERS-CoV CDRL3
QQYYSVPFT

SEQ ID NO: 150 - protein, 8 aa, Artificial Sequence
  anti-MERS-CoV CDRH1
GYTFNVYA

SEQ ID NO: 151 - protein, 8 aa, Artificial Sequence
  anti-MERS-CoV CDRH2
IIPILGIA

SEQ ID NO: 152 - protein, 14 aa, Artificial Sequence
  anti-MERS-CoV CDRH3
ARDYYGSGARGFDY

SEQ ID NO: 153 - protein, 8 aa, Artificial Sequence
  anti-MERS-CoV CDRL1
SNNVGNQG

SEQ ID NO: 154 - protein, 11 aa, Artificial Sequence
  anti-MERS-CoV CDRL3
ASWDSSLSVWV

SEQ ID NO: 155 - skipped

SEQ ID NO: 156 - protein, 8 aa, Artificial Sequence
  anti-MERS-CoV CDRH2
IIPIFGIA

SEQ ID NO: 157 - protein, 19 aa, Artificial Sequence
  anti-MERS-CoV CDRH3
ASSNYYGSGSYYPRSAFDI

SEQ ID NO: 158 - protein, 6 aa, Artificial Sequence
  anti-MERS-CoV CDRL1
QSISND

SEQ ID NO: 159 - protein, 9 aa, Artificial Sequence
  anti-MERS-CoV CDRL3
QQYGVSPLT

SEQ ID NO: 160 - protein, 113 aa, Artificial Sequence
  anti-SARS-CoV variable heavy chain
QVQLVQSGAEVKKPGASVKVSCKASGYTFTSYGISWVRQAPGQGLEWMGWISAYNGNTNYAQKLQGRVTMTTDTSTNTAYMELRSLRSDDTAVYYCAVGRYLDYWGQGTLVTV

SEQ ID NO: 161 - protein, 107 aa, Artificial Sequence
  anti-SARS-CoV variable light chain
DIQMTQSPSSLSASVGDRVTITCRASQDIRNDLGWYQQKPGKAPKRLIYAASSLQSGVPSRFSGSGSGTEFTLTISSLQPEDFATYYCLQYNSYPLTFGGGTKVEIK

SEQ ID NO: 162 - protein, 115 aa, Artificial Sequence
  anti-SARS-CoV variable heavy chain
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAVGRYLDYWGQGTLVTVSS

SEQ ID NO: 163 - skipped

SEQ ID NO: 164 - protein, 115 aa, Artificial Sequence
  anti-SARS-CoV variable heavy chain
QVHLVQSGAEVKKPGASVKVSCKASGYTFTGYYVHWVRQAPGQGLEWMGWINPNSGGTNYAQKFQGRVTMTRDTSISTAYMELSRLRSDDTAVYFCAGGRYLDYWGQGTLVTVSS

SEQ ID NO: 165 - protein, 107 aa, Artificial Sequence
  anti-SARS-CoV variable light chain
DIQMTQSPSSLSASVGDRVTITCRASQGIRNDLGWYQQKSGKAPKRLIYAASSLQSGVPSRFSGSGSGTEFTLTISSLQPEDFATYYCLQYNSYPITFGQGTRLEIK

SEQ ID NO: 166 - protein, 126 aa, Artificial Sequence
  PI3-E12 heavy chain
QVQLLESGGKLVKPGGSLRLSCAASGFTFSDHYMIWIRQAPGKGLEWISYISSSGSNTIYADSLMGRFTISRDNAKNSLYLQMNSLRTEDTAVYYCARAKWGTMGRGAPPTIYDHWGQGTLVTVSS

SEQ ID NO: 167 - protein, 112 aa, Artificial Sequence
  PI3-E12 light chain
DIVMTQSPLSLPVTPGEPASISCRSSQSLLQSNGNNYLEWYLQKPGQSPQLLIYLGSNRASGVPDRFSGSGSGTDFTLKISRVEAEDAGVYYCMQALQTPLTFGGGTKVEIK

SEQ ID NO: 168 - protein, 122 aa, Artificial Sequence
  mAb PI3-A3 heavy chain
EVQLVESGGGLVQPGGSLRLSCTASGFTFSNYWMSWVRQAPGKGLEWVANVKEEGSEKHYVDSVKGRFTISRDNAKNSVYLQMSSLRAEDTAVYYCAGEVKSGWFGRYFDSWGQGTLVTVSS

SEQ ID NO: 169 - protein, 107 aa, Artificial Sequence
  mAb PI3-A3 light chain
DIQMTQSPSTLSASVGDRVTINCRASQSVGSWLAWYQQKPGKAPKLLMYKTSTLQRGVPSRFSGSGSGTEFTLTISSLQPDDFAAYYCQQYSSFPYTFGQGTKLEIK

SEQ ID NO: 170 - protein, 122 aa, Artificial Sequence
  mAb PI3-B5 heavy chain
EVQLVQSGAEVKKPGESLRISCKGSGYNFTNYWIAWVRQMPGKGLEWMGIIYPADSDTRYSPSFQGQVTISADKSITTAYLQWSSLKASDTAIYYCARPSTRWFVPGGMDVWGQGTTVIVSS

SEQ ID NO: 171 - protein, 107 aa, Artificial Sequence
  mAb PI3-B5 light chain
DIQMTQSPSTLSASVGDRVTISCRATQSIGAWLAWYQQKPGEPPKLLIYKASTLESGVPSRFSGSGSGTEFTLTISSLQPDDSATYYCQQHSSYPSTFGGGTKVEIK

SEQ ID NO: 172 - protein, 119 aa, Artificial Sequence
  mAb PI3-A10 heavy chain
QVQLVESGGGVVRPGRSLRLSCVASGFNFNNYGLQWIRQAPGKGLEWVAGVSFDGSNRYYADSVKGRVTISRDDSKNTLYLEMNSLRAEDTGIYYCSKSKYSDFWSEIWGQGTLVTVSS

SEQ ID NO: 173 - protein, 107 aa, Artificial Sequence
  mAb PI3-A10 light chain
EIVLTQSPATLSLSPGERATLSCRASQNVMRYLAWYQQRPGQAPRLLFYDASSRATGIPARFTASGSGTDFTLTISGLEPGDFAVYYCQQRTNHRFSFGPGTKVDIK

SEQ ID NO: 174 - protein, 123 aa, Artificial Sequence
  mAb PI3-A12 heavy chain
QVQLQESGPGLVKPSETLSLTCTVSGDSVKSDDFYWSWIRQPPGKGLEWIGFIYYGGRTYYNPSLSGRGTISVDTSKNHFFLELTSVTAADTAVYYCVRVEGLLWFGELFDYWGQGTLVTVSS

SEQ ID NO: 175 - protein, 110 aa, Artificial Sequence
  mAb PI3-A12 light chain
QSVLTQPPSASGTPGQRVTISCSGSNSNIGNNFVYWYQQVPGSAPKVVIYKDYQRPSGVPDRFSASKSGTSASLTISGLRSDDEAHYYCAAWQDGLSGPLFGGGTKLTVL

SEQ ID NO: 176 - protein, 128 aa, Artificial Sequence
  mAb 3x1 heavy chain
EVQLLESGGGLVQPGGSLRLSCAASGFTFSSFGMSWVRQSPGKGLEWVADISHSAGFLNYADSVKGRFTVSRDNSKSTLHLQMKSLRAEDTAVYYCAKRLAGLPDLEWLLYPNFLDHWGQGTLVTVSS

SEQ ID NO: 177 - protein, 108 aa, Artificial Sequence
  mAb 3x1 light chain
SSELTQDPAVSVALGQTVRITCQGDILRTYYVSWYQQKPGQAPLLVIYGKNNRPSVIPDRFSGSTSGDTASLTITGAQAEDEAEYYCSSRDRSGNHVLFGGGTKLTVL

SEQ ID NO: 178 - protein, 120 aa, Artificial Sequence
  MxR-B11 variable heavy chain
EVQVVESGGGLVKPGGSLRLSCAASGFPFSSYKMDWVRQAPGKGLEWVSSISASGSYINYADSVKGRFTISRDNAKNSLYLQMKSLRADDTAVYFCARDGGRELSPFEKWGQGILVTVSS

SEQ ID NO: 179 - protein, 111 aa, Artificial Sequence
  MxR-B11 variable light chain
QSVLTQPPSVSGAPGQRVTISCTGTNSNIGTGYDVHWYQQLPGTAPKVVLFDNNNRPSGVPDRFSGSKSGTSAALAITGLQAEDEAVYYCQSYDKSLGGWVFGGGTKLTVL

SEQ ID NO: 180 - protein, 120 aa, Artificial Sequence
  mAb MxR-D10 variable heavy chain
EVQVVESGGGLVKPGGSLRLSCTASGFIFSNYDMNWVRQAPGKGLEWVASITGGSSFINYADSVKGRFTISRDNAKKSLYLQMNSLRAEDTAVYYCARDGGRQLSPCEHWGQGTLVTVSS

SEQ ID NO: 181 - protein, 111 aa, Artificial Sequence
  mAb MxR-D10 variable light chain
QSVLTQPPSVSGSPGQRVTISCTGGSSNIGAGYDVHWYQQLPGSAPKLLMYDSNNRPSGVPDRFSGSKSGTSASLAITGLQAEDEADYYCQSYDRGLSGWAFGGGTKLTVL

SEQ ID NO: 182 - protein, 9 aa, Artificial Sequence
  PIA174 CDRH1
GGSVSSSYY

SEQ ID NO: 183 - protein, 7 aa, Artificial Sequence
  PIA174 CDRH2
IYYSGTT

SEQ ID NO: 184 - protein, 15 aa, Artificial Sequence
  PIA174 CDRH3
ARQVKSGWFVQPFDY

SEQ ID NO: 185 - protein, 6 aa, Artificial Sequence
  PIA174 CDRL1
QAIANY

SEQ ID NO: 186 - protein, 9 aa, Artificial Sequence
  PIA174 CDRL3
HQYNTYPIT

SEQ ID NO: 187 - DNA, 22 bp, Artificial Sequence
  Reverse transcription primer for RSV
tccagcaaatacaccatccaac

SEQ ID NO: 188 - DNA, 25 bp, Artificial Sequence
  Reverse transcription primer for HPIV3
ctagaaggtcaagaaaagggaactc

SEQ ID NO: 189 - DNA, 25 bp, Artificial Sequence
  RSV forward primer
tgactctcctgattgtgggatgata

SEQ ID NO: 190 - DNA, 21 bp, Artificial Sequence
  RSV reverse primer
cggctgtaagaccagatctgt

SEQ ID NO: 191 - DNA, 16 bp, Artificial Sequence
  RSV reporter
cccctgctgctaattt

SEQ ID NO: 192 - DNA, 22 bp, Artificial Sequence
  HPIV3 forward primer
cggtgacacagtggatcagatt

SEQ ID NO: 193 - DNA, 27 bp, Artificial Sequence
  HPIV3 reverse primer
tgtttcaaccataagagttaccaagct

SEQ ID NO: 194 - DNA, 16 bp, Artificial Sequence
  HPIV3 reporter
accgcatgattgaccc